(19) United States
(12) Patent Application Publication
(10) Pub. No.: US 2024/0294495 A1
(43) Pub. Date: Sep. 5, 2024

(54) COMPOUNDS THAT MEDIATE PROTEIN DEGRADATION AND METHODS OF USE THEREOF

(71) Applicant: Monte Rosa Therapeutics, Inc., Boston, MA (US)

(72) Inventors: Bernhard Fasching, Basel (CH); Thomas Ryckmans, Basel (CH); Alexander Flohr, Basel (CH); Oliv Eidam, Basel (CH); Andreas Ritzén, Basel (CH)

(21) Appl. No.: 18/640,909

(22) Filed: Apr. 19, 2024

Related U.S. Application Data
(63) Continuation of application No. PCT/US2022/047454, filed on Oct. 21, 2022.
(60) Provisional application No. 63/262,923, filed on Oct. 22, 2021.

(51) Int. Cl. C07D 401/10; A61K 31/4523; A61K 31/4525; A61K 31/4545; A61K 31/5377; C07D 401/14; C07D 405/14

(52) U.S. Cl. CPC C07D 401/10; A61K 31/4523; A61K 31/4525; A61K 31/4545; A61K 31/5377; C07D 401/14; C07D 405/14

(57) ABSTRACT

Described herein, in part, are compounds that bind to and modulate the surface of cereblon and mediate the degradation of GSPT1, and are therefore useful in the treatment of various disorders, such as cancer.

### CROSS-REFERENCE TO RELATED APPLICATIONS

This application is a continuation of International Application Number PCT/US2022/047454, filed on Oct. 21, 2022, which claims the benefit of, and priority to U.S. Provisional Application No. 63/262,923, filed Oct. 22, 2021, the contents of which are incorporated herein by reference.

### BACKGROUND

The ubiquitin proteasome system can be manipulated with different small molecules to trigger targeted degradation of specific proteins of interest. Promoting the targeted degradation of pathogenic proteins using small molecule degraders is emerging as a new modality in the treatment of diseases. One such modality relies on redirecting the activity of E3 ligases such as cereblon (a phenomenon known as E3 reprogramming) using low molecular weight compounds, which have been termed molecular glues to promote the poly-ubiquitination and ultimately proteasomal degradation of new protein substrates involved in the development of diseases. The molecular glues bind to both the E3 ligase and the target protein, thereby mediating an alteration of the ligase surface and enabling an interaction with the target protein.

There exists a need for therapeutics that effectively mediate the degradation of certain proteins for the treatment of diseases.

### SUMMARY

Described herein, in part, are compounds contemplated as binders of cereblon that mediate the degradation of a protein, and are therefore useful in the treatment of disorders, such as cancer. For example, it has been found that compounds of the present disclosure bind to and modulate the surface of cereblon to subsequently mediate the targeted degradation of the protein GSPT1.

In one aspect, described herein is a compound of Formula (I):

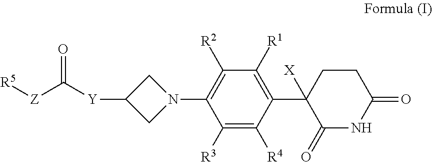

or a pharmaceutically acceptable salt thereof, wherein: X is H or deuterium; each of Y and Z is independently C(R^{A})₂, NH, or C₃₋₁₀ cycloalkyl, provided that, when Y is NH, Z is CR^{A} or C₃₋₁₀ cycloalkyl, and when Y is CR^{A} or C₃₋₁₀ cycloalkyl, then Z is NH; each of R¹, R², R³, and R⁴ is independently H or halogen; R⁵ is C₁₋₆ alkyl, C₂₋₆ alkenyl, C₂₋₆ alkynyl, C₃₋₁₀ cycloalkyl, 3 to 10 membered heterocyclyl, aryl, or heteroaryl, wherein each of C₁₋₆ alkyl, C₂₋₆ alkenyl, C₂₋₆ alkynyl, C₃₋₁₀ cycloalkyl, aryl, and heteroaryl is optionally substituted with one or more substituents each independently selected from R⁶; each occurrence of R⁶ is independently halogen, cyano, C₁₋₆ alkyl, C₁₋₆ alkoxy, C₃₋₁₀ cycloalkyl, 3 to 10 membered heterocyclyl, aryl, heteroaryl, —C(O)OR^{B}, or —OC(O)R^{C}, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more substituents each independently selected from R⁷, or two R⁶ are joined to together to form a 3 to 10-membered ring optionally substituted with one or more substituents each independently selected from R⁷; each occurrence of R⁷ is independently halogen, C₁₋₆ alkyl, 3 to 10 membered heterocyclyl, or aryl, wherein each of C₁₋₄ alkyl, 3 to 10 membered heterocyclyl, and aryl is optionally substituted with R⁸; R⁸ is C₁₋₆ alkoxy; and each occurrence of R^{A}, R^{B}, and R^{C} is independently selected from the group consisting of H, C₁₋₆ alkyl, and halogen.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Compounds

In one aspect, described herein is a compound of Formula (I):

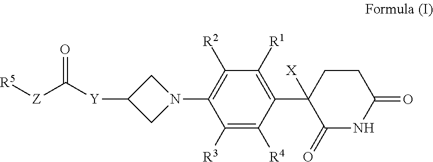

or a pharmaceutically acceptable salt thereof, wherein: X is H or deuterium; each of Y and Z is independently C(R^{A})₂, NH, or C₃₋₁₀ cycloalkyl, provided that, when Y is NH, Z is CR^{A} or C₃₋₁₀ cycloalkyl, and when Y is CR^{A} or C₃₋₁₀ cycloalkyl, then Z is NH; each of R¹, R², R³, and R⁴ is independently H or halogen; R⁵ is C₁₋₆ alkyl, C₂₋₆ alkenyl, C₂₋₆ alkynyl, C₃₋₁₀ cycloalkyl, 3 to 10 membered heterocyclyl, aryl, or heteroaryl, wherein each of C₁₋₆ alkyl, C₂₋₆ alkenyl, C₂₋₆ alkynyl, C₃₋₁₀ cycloalkyl, aryl, and heteroaryl is optionally substituted with one or more substituents each independently selected from R⁶; each occurrence of R⁶ is independently halogen, cyano, C₁₋₆ alkyl, C₁₋₆ alkoxy, C₃₋₁₀ cycloalkyl, 3 to 10 membered heterocyclyl, aryl, heteroaryl, —C(O)OR^{B}, or —OC(O)R^{C}, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more substituents each independently selected from R⁷, or two R⁶ are joined to together to form a 3 to 10-membered ring optionally substituted with one or more substituents each independently selected from R⁷; each occurrence of R⁷ is independently halogen, C₁₋₆ alkyl, 3 to 10 membered heterocyclyl, or aryl, wherein each of C₁₋₆ alkyl, 3 to 10 membered heterocyclyl, and aryl is optionally substituted with R⁸; R⁸ is C₁₋₆ alkoxy; and each occurrence of R^{A}, R^{B}, and R^{C} is independently selected from the group consisting of H, C₁₋₆ alkyl, and halogen.

In another aspect, provided herein is a pharmaceutical composition comprising a compound described herein, or pharmaceutically acceptable salt thereof, and a pharmaceutically acceptable excipient.

In another aspect, described herein is a method of degrading GSPT1 in a subject suffering from cancer, comprising administering to the subject an effective amount of a compound described herein, or pharmaceutically acceptable salt thereof, or a pharmaceutical composition described herein.

In another aspect, described herein is a method of treating cancer in a subject in need thereof, comprising administering to the subject a therapeutically effective amount of a compound described herein, or pharmaceutically acceptable salt thereof, or a pharmaceutical composition described herein.

In another aspect, described herein is a method of treating a solid tumor in a subject in need thereof, comprising administering to the subject a therapeutically effective amount of a compound described herein, or pharmaceutically acceptable salt thereof, or a pharmaceutical composition described herein.

In another aspect, described herein is a method of treating a liquid tumor in a subject in need thereof, comprising administering to the subject a therapeutically effective amount of a compound described herein, or pharmaceutically acceptable salt thereof, or a pharmaceutical composition described herein.

In some embodiments, the compound is a compound of Formula (I-A):

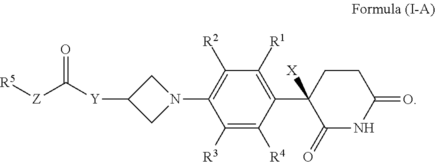

In some embodiments, the compound is a compound of Formula (I-B):

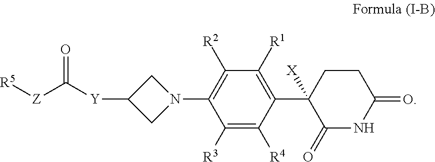

In some embodiments, X is H. In some embodiments, Y is NH. In some embodiments, Z is CH₂. In some embodiments, Z is CF₂. In some embodiments, R¹ and R⁴ are halogen. In some embodiments, R¹ and R⁴ are fluorine. In some embodiments, R² and R³ are H. In some embodiments, R⁵ is C₃₋₁₀ cycloalkyl, C₆₋₁₀ aryl, or 5-membered heteroaryl, or 6-membered heteroaryl.

In some embodiments, R⁵ is:

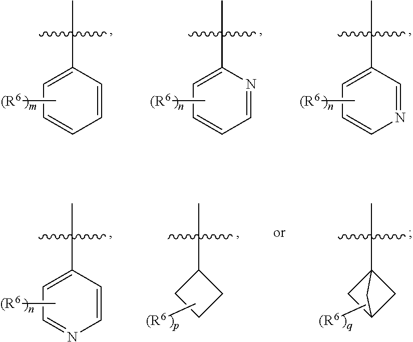

wherein m is an integer from 0 to 5, each occurrence of n is independently an integer from 0 to 4, p is an integer from 0 to 3, and q is an integer from 0 to 1. In some embodiments, R⁶ is halogen, cyano, C₁₋₆ alkyl, or C₁₋₆ alkoxy, wherein each of C₁₋₆ alkyl or C₁₋₆ alkoxy is optionally substituted with one or more occurrences of halogen. In some embodiments, R⁶ is chlorine, cyano, —CH₃, or —OCH₃. In some embodiments, R⁶ is chlorine, cyano, or —CH₃. In some embodiments, R⁷ is halogen or C₁₋₆ alkyl. In some embodiments, R⁷ is chlorine or —CH₃. In some embodiments, m is 1, 2, or 3. In some embodiments, each occurrence of n is 1, 2, or 3. In some embodiments, p is 1. In some embodiments, each occurrence of R^{A} is H. In some embodiments, each occurrence of R^{A} is F.

In another aspect, described herein is a compound of Formula (II):

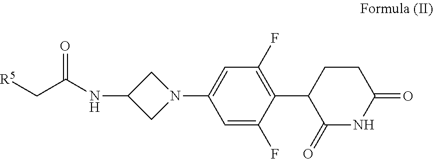

or a pharmaceutically acceptable thereof, wherein: R⁵ is heteroaryl, aryl, or C₃₋₁₀ cycloalkyl, wherein each of heteroaryl, aryl, and C₃₋₁₀ cycloalkyl is optionally substituted with one or more substituents each independently selected from R⁶; each occurrence of R⁶ is independently halogen, cyano, C₁₋₆ alkyl, C₁₋₆ alkoxy, C₃₋₁₀ cycloalkyl, 3 to 10 membered heterocyclyl, phenyl, or 5-membered heteroaryl, or 6-membered heteroaryl, wherein each of C₁₋₆ alkyl, C₁₋₆ alkoxy, C₃₋₁₀ cycloalkyl, 3 to 10 membered heterocyclyl, phenyl, 5-membered heteroaryl, and 6-membered heteroaryl is optionally substituted with one or more substituents each independently selected from R⁷; and each occurrence of R⁷ is independently halogen, or C₁₋₆ alkyl.

In some embodiments, the compound is a compound of Formula (II-A):

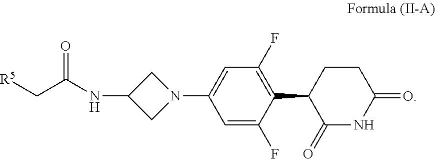

In some embodiments, the compound is a compound of Formula (II-B):

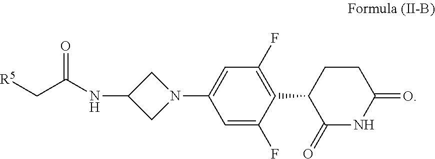

In some embodiments, R⁵ is C₃₋₁₀ cycloalkyl, C₆₋₁₀ aryl, or 5 or 6-membered heteroaryl.

In some embodiments, R⁵ is:

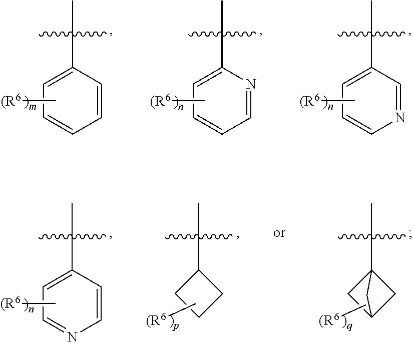

wherein m is an integer from 0 to 5, each occurrence of n is independently an integer from 0 to 4, p is an integer from 0 to 3, and q is an integer from 0 to 1. In some embodiments, R⁶ is halogen, cyano, C₁₋₆ alkyl, or C₁₋₆ alkoxy, wherein each of C₁₋₆ alkyl or C₁₋₆ alkoxy is optionally substituted with one or more occurrences of halogen. In some embodiments, R⁶ is chlorine, cyano, —CH₃, or —OCH₃. In some embodiments, R⁶ is chlorine, cyano, or —CH₃. In some embodiments, R⁷ is halogen or C₁₋₆ alkyl. In some embodiments, R⁷ is chlorine or —CH₃. In some embodiments, m is 1, 2, or 3. In some embodiments, each occurrence of n is independently 1, 2, or 3. In some embodiments, p is 1.

In another aspect, described herein is a compound of Formula (III):

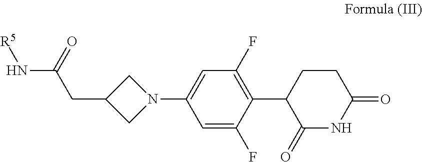

or a pharmaceutically acceptable thereof, wherein: R⁵ is heteroaryl, aryl, or C₃₋₁₀ cycloalkyl, wherein each of heteroaryl, aryl, and C₃₋₁₀ cycloalkyl is optionally substituted with one or more substituents each independently selected from R⁶; each occurrence of R⁶ is independently halogen, cyano, C₁₋₆ alkyl, C₁₋₆ alkoxy, C₃₋₁₀ cycloalkyl, 3 to 10 membered heterocyclyl, phenyl, or 5-membered heteroaryl, or 6-membered heteroaryl, wherein each of C₁₋₆ alkyl, C₁₋₄ alkoxy, C₃₋₁₀ cycloalkyl, 3 to 10 membered heterocyclyl, phenyl, 5-membered heteroaryl, and 6-membered heteroaryl is optionally substituted with one or more substituents each independently selected from R⁷; and each occurrence of R⁷ is independently halogen, or C₁₋₆ alkyl.

In some embodiments, the compound is a compound of Formula (III-A):

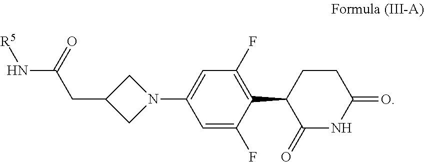

In some embodiments, the compound is a compound of Formula (III-B):

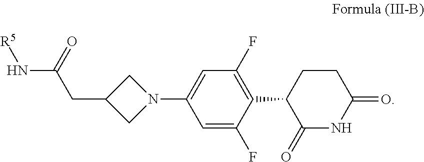

In some embodiments, R⁵ is C₃₋₁₀ cycloalkyl, C₆₋₁₀ aryl, 5-membered heteroaryl, or 6-membered heteroaryl.

In some embodiments, R⁵ is:

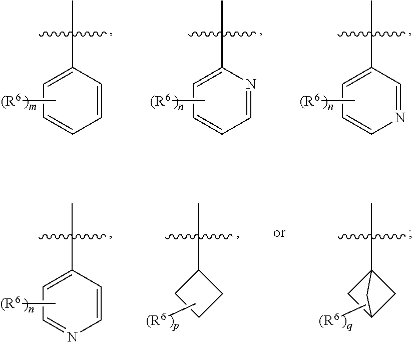

wherein m is an integer from 0 to 5, each occurrence of n is independently an integer from 0 to 4, p is an integer from 0 to 3, and q is an integer from 0 to 1. In some embodiments, R⁶ is halogen, cyano, C₁₋₆ alkyl, or C₁₋₆ alkoxy, wherein each of C₁₋₆ alkyl or C₁₋₆ alkoxy is optionally substituted with one or more occurrences of halogen. In some embodiments, R⁶ is chlorine, cyano, —CH₃, or —OCH₃. In some embodiments, R⁶ is chlorine, cyano, or —CH₃. In some embodiments, R⁷ is halogen or C₁₋₆ alkyl. In some embodiments, R⁷ is chlorine or —CH₃. In some embodiments, m is 1, 2, or 3. In some embodiments, each occurrence of n is independently 1, 2, or 3. In some embodiments, p is 1.

In some embodiments, the compound is a compound described in Table 1 below. Table 1 also includes the compound number of each compound in accordance with the contents of the present specification.

TABLE 1
Exemplary Compounds.

| No. | Compound |
|---|---|
| 1 | 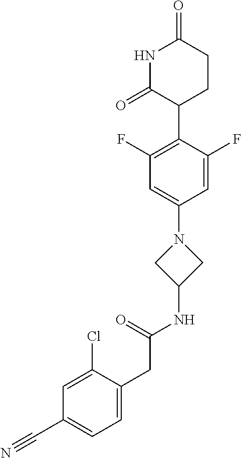 [SMILES: N#Cc1ccc(CC(=O)NC2CN(c3cc(F)c(C4CCC(=O)NC4=O)c(F)c3)C2)c(Cl)c1] |
| 2 | 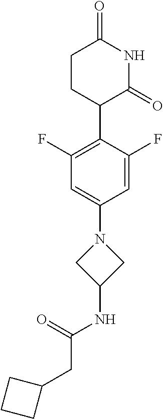 [SMILES: O=C1CCC(c2c(F)cc(N3CC(NC(=O)CC4CCC4)C3)cc2F)C(=O)N1] |
| 3 | 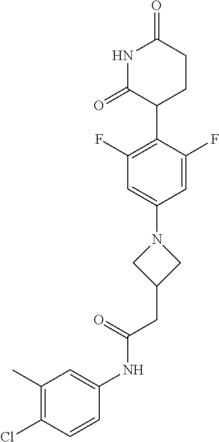 [SMILES: Cc1cc(NC(=O)CC2CN(c3cc(F)c(C4CCC(=O)NC4=O)c(F)c3)C2)ccc1Cl] |
| 4 | 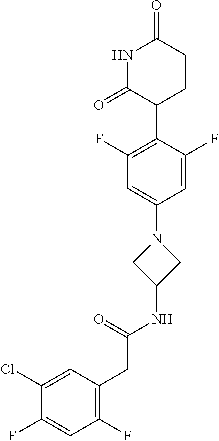 [SMILES: O=C1CCC(c2c(F)cc(N3CC(NC(=O)Cc4cc(Cl)c(F)cc4F)C3)cc2F)C(=O)N1] |
| 5 | 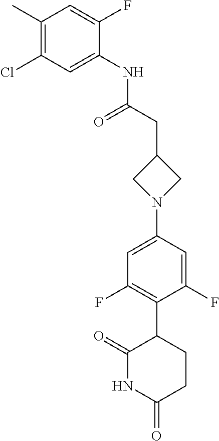 [SMILES: Cc1cc(F)c(NC(=O)CC2CN(c3cc(F)c(C4CCC(=O)NC4=O)c(F)c3)C2)cc1Cl] |
| 6 | 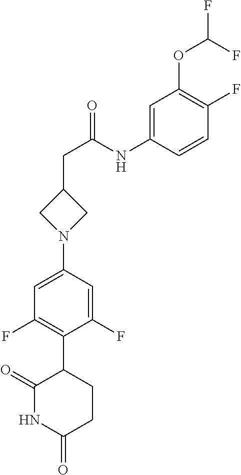 [SMILES: O=C1CCC(c2c(F)cc(N3CC(CC(=O)Nc4ccc(F)c(OC(F)F)c4)C3)cc2F)C(=O)N1] |
| 7 | 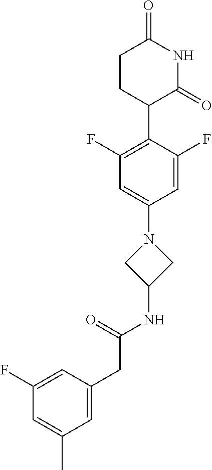 [SMILES: Cc1cc(F)cc(CC(=O)NC2CN(c3cc(F)c(C4CCC(=O)NC4=O)c(F)c3)C2)c1] |
| 8 | 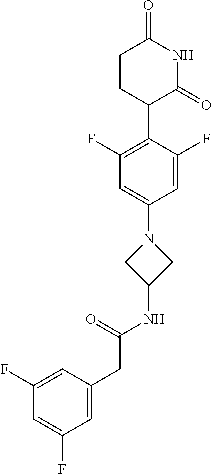 [SMILES: O=C1CCC(c2c(F)cc(N3CC(NC(=O)Cc4cc(F)cc(F)c4)C3)cc2F)C(=O)N1] |
| 9 | 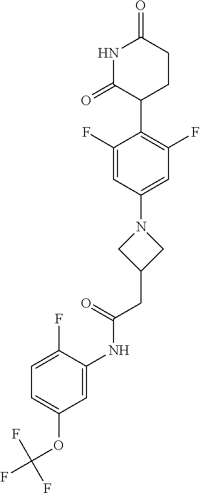 [SMILES: O=C1CCC(c2c(F)cc(N3CC(CC(=O)Nc4cc(OC(F)(F)F)ccc4F)C3)cc2F)C(=O)N1] |
| 10 | 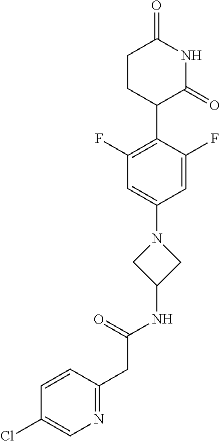 [SMILES: O=C1CCC(c2c(F)cc(N3CC(NC(=O)Cc4ccc(Cl)cn4)C3)cc2F)C(=O)N1] |
| 11 | 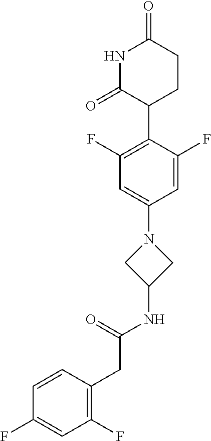 [SMILES: O=C1CCC(c2c(F)cc(N3CC(NC(=O)Cc4ccc(F)cc4F)C3)cc2F)C(=O)N1] |
| 12 | 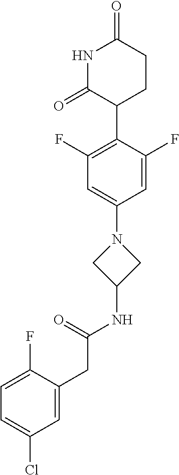 [SMILES: O=C1CCC(c2c(F)cc(N3CC(NC(=O)Cc4cc(Cl)ccc4F)C3)cc2F)C(=O)N1] |
| 13 | 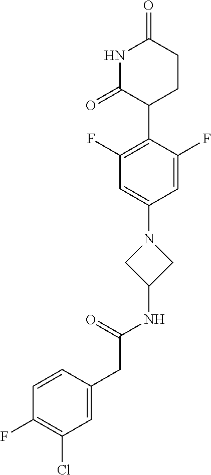 [SMILES: O=C1CCC(c2c(F)cc(N3CC(NC(=O)Cc4ccc(F)c(Cl)c4)C3)cc2F)C(=O)N1] |
| 14 | 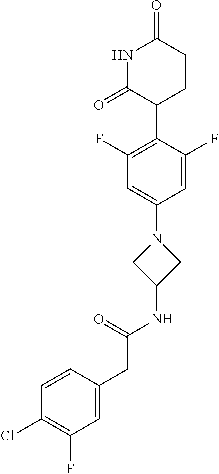 [SMILES: O=C1CCC(c2c(F)cc(N3CC(NC(=O)Cc4ccc(Cl)c(F)c4)C3)cc2F)C(=O)N1] |
| 15 | 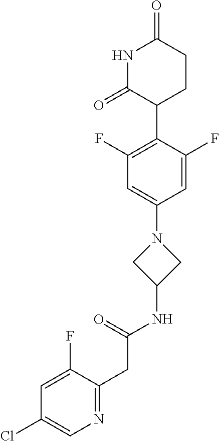 [SMILES: O=C1CCC(c2c(F)cc(N3CC(NC(=O)Cc4ncc(Cl)cc4F)C3)cc2F)C(=O)N1] |
| 16 | 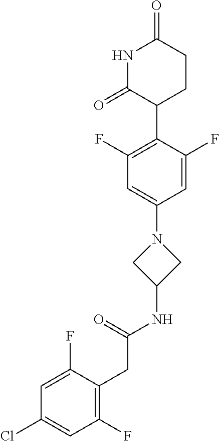 [SMILES: O=C1CCC(c2c(F)cc(N3CC(NC(=O)Cc4c(F)cc(Cl)cc4F)C3)cc2F)C(=O)N1] |
| 17 | 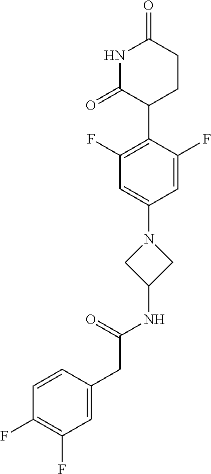 [SMILES: O=C1CCC(c2c(F)cc(N3CC(NC(=O)Cc4ccc(F)c(F)c4)C3)cc2F)C(=O)N1] |
| 18 | 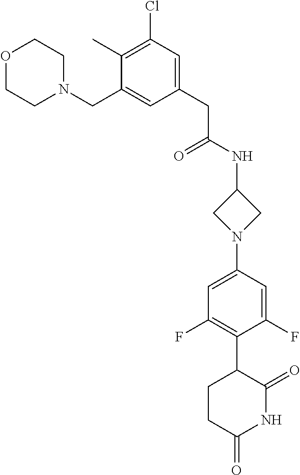 [SMILES: Cc1c(Cl)cc(CC(=O)NC2CN(c3cc(F)c(C4CCC(=O)NC4=O)c(F)c3)C2)cc1CN1CCOCC1] |
| 19 | 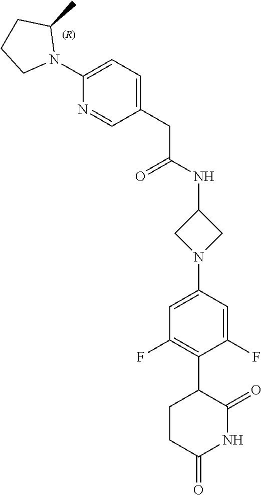 |
| 20 | 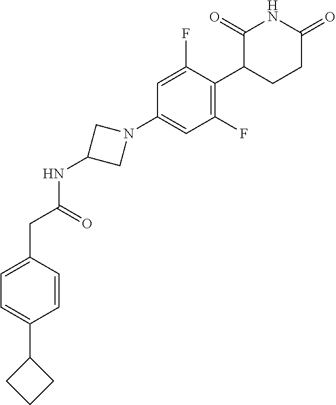 [SMILES: O=C1CCC(c2c(F)cc(N3CC(NC(=O)Cc4ccc(C5CCC5)cc4)C3)cc2F)C(=O)N1] |
| 21 | 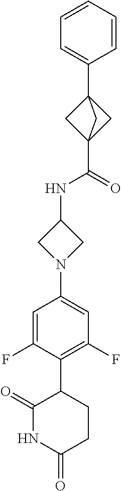 [SMILES: O=C1CCC(c2c(F)cc(N3CC(NC(=O)C45CC(c6ccccc6)(C4)C5)C3)cc2F)C(=O)N1] |
| 22 | 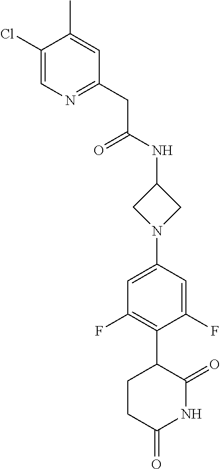 [SMILES: Cc1cc(CC(=O)NC2CN(c3cc(F)c(C4CCC(=O)NC4=O)c(F)c3)C2)ncc1Cl] |
| 23 | 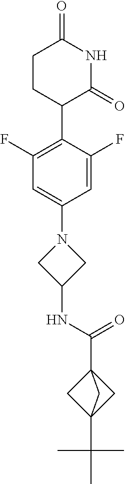 [SMILES: CC(C)(C)C12CC(C(=O)NC3CN(c4cc(F)c(C5CCC(=O)NC5=O)c(F)c4)C3)(C1)C2] |
| 24 | 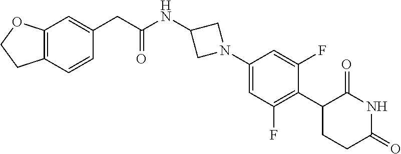 [SMILES: O=C1CCC(c2c(F)cc(N3CC(NC(=O)Cc4ccc5c(c4)OCC5)C3)cc2F)C(=O)N1] |
| 25 | 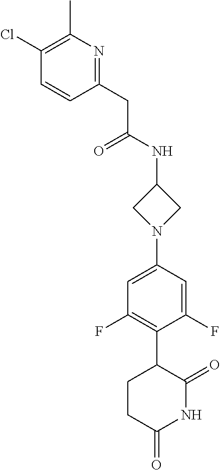 [SMILES: Cc1nc(CC(=O)NC2CN(c3cc(F)c(C4CCC(=O)NC4=O)c(F)c3)C2)ccc1Cl] |
| 26 | 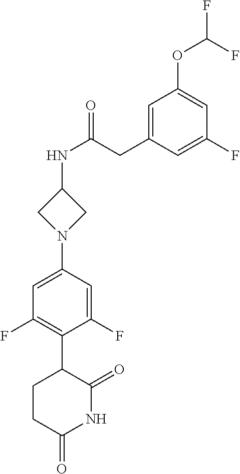 [SMILES: O=C1CCC(c2c(F)cc(N3CC(NC(=O)Cc4cc(F)cc(OC(F)F)c4)C3)cc2F)C(=O)N1] |
| 27 | 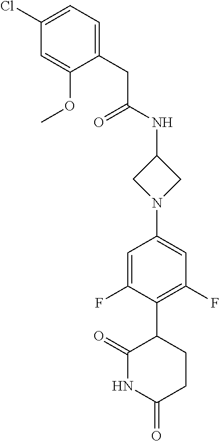 [SMILES: COc1cc(Cl)ccc1CC(=O)NC1CN(c2cc(F)c(C3CCC(=O)NC3=O)c(F)c2)C1] |
| 28 | 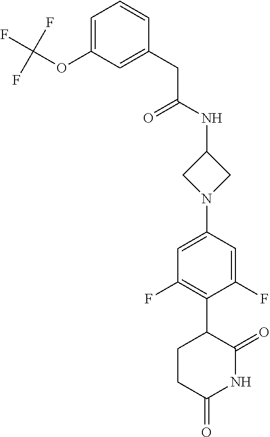 [SMILES: O=C1CCC(c2c(F)cc(N3CC(NC(=O)Cc4cccc(OC(F)(F)F)c4)C3)cc2F)C(=O)N1] |
| 29 | 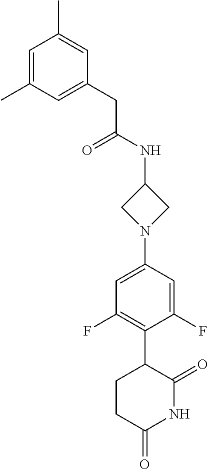 [SMILES: Cc1cc(C)cc(CC(=O)NC2CN(c3cc(F)c(C4CCC(=O)NC4=O)c(F)c3)C2)c1] |
| 30 | 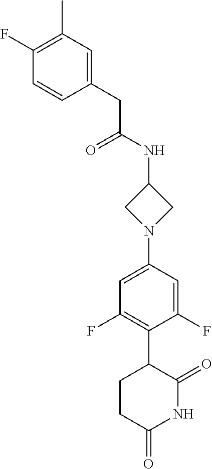 [SMILES: Cc1cc(CC(=O)NC2CN(c3cc(F)c(C4CCC(=O)NC4=O)c(F)c3)C2)ccc1F] |
| 31 | 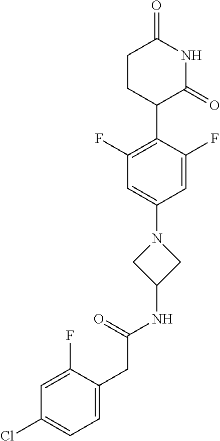 [SMILES: O=C1CCC(c2c(F)cc(N3CC(NC(=O)Cc4ccc(Cl)cc4F)C3)cc2F)C(=O)N1] |
| 32 | 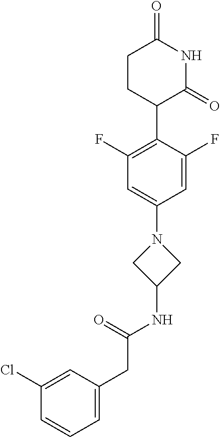 [SMILES: O=C1CCC(c2c(F)cc(N3CC(NC(=O)Cc4cccc(Cl)c4)C3)cc2F)C(=O)N1] |
| 33 | 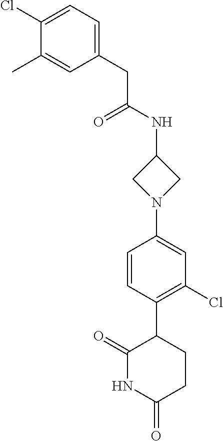 [SMILES: Cc1cc(CC(=O)NC2CN(c3ccc(C4CCC(=O)NC4=O)c(Cl)c3)C2)ccc1Cl] |
| 34 | 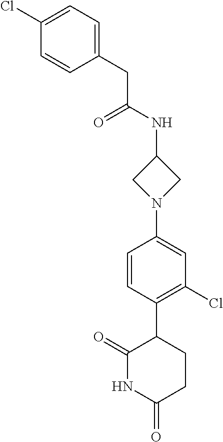 [SMILES: O=C1CCC(c2ccc(N3CC(NC(=O)Cc4ccc(Cl)cc4)C3)cc2Cl)C(=O)N1] |
| 35 | 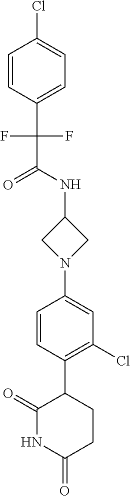 [SMILES: O=C1CCC(c2ccc(N3CC(NC(=O)C(F)(F)c4ccc(Cl)cc4)C3)cc2Cl)C(=O)N1] |
| 36 | 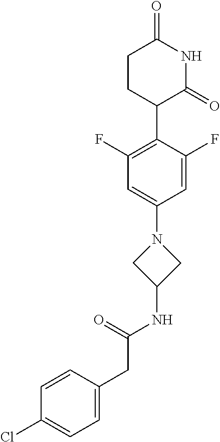 [SMILES: O=C1CCC(c2c(F)cc(N3CC(NC(=O)Cc4ccc(Cl)cc4)C3)cc2F)C(=O)N1] |
| 37 | 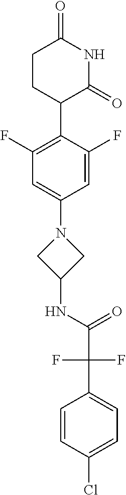 [SMILES: O=C1CCC(c2c(F)cc(N3CC(NC(=O)C(F)(F)c4ccc(Cl)cc4)C3)cc2F)C(=O)N1] |
| 38 | 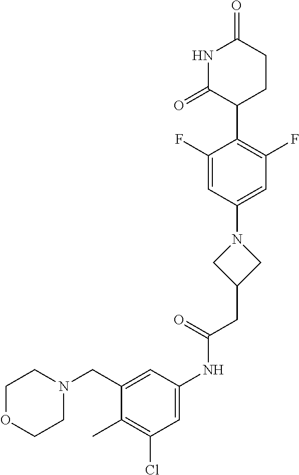 [SMILES: Cc1c(Cl)cc(NC(=O)CC2CN(c3cc(F)c(C4CCC(=O)NC4=O)c(F)c3)C2)cc1CN1CCOCC1] |
| 39 | 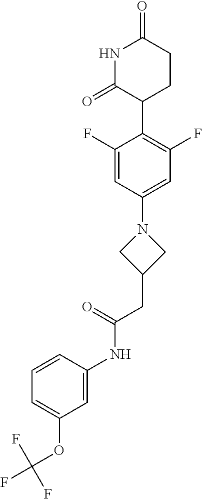 [SMILES: O=C1CCC(c2c(F)cc(N3CC(CC(=O)Nc4cccc(OC(F)(F)F)c4)C3)cc2F)C(=O)N1] |
| 40 | 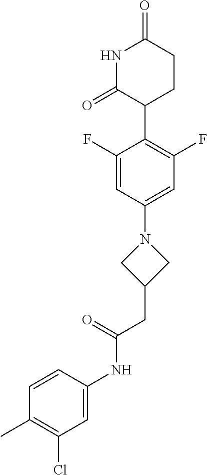 [SMILES: Cc1ccc(NC(=O)CC2CN(c3cc(F)c(C4CCC(=O)NC4=O)c(F)c3)C2)cc1Cl] |
| 41 | 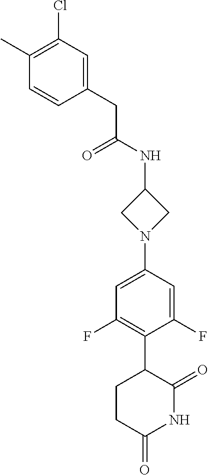 [SMILES: Cc1ccc(CC(=O)NC2CN(c3cc(F)c(C4CCC(=O)NC4=O)c(F)c3)C2)cc1Cl] |
| 42 | 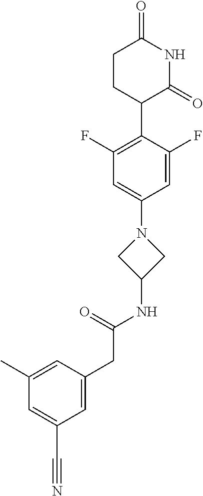 [SMILES: Cc1cc(C#N)cc(CC(=O)NC2CN(c3cc(F)c(C4CCC(=O)NC4=O)c(F)c3)C2)c1] |
| 43 | 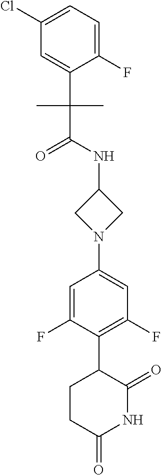 [SMILES: CC(C)(C(=O)NC1CN(c2cc(F)c(C3CCC(=O)NC3=O)c(F)c2)C1)c1cc(Cl)ccc1F] |
| 44 | 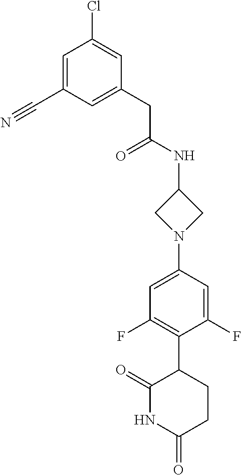 [SMILES: N#Cc1cc(Cl)cc(CC(=O)NC2CN(c3cc(F)c(C4CCC(=O)NC4=O)c(F)c3)C2)c1] |
| 45 | 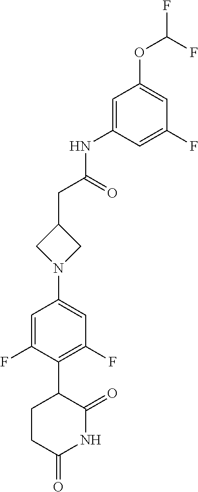 [SMILES: O=C1CCC(c2c(F)cc(N3CC(CC(=O)Nc4cc(F)cc(OC(F)F)c4)C3)cc2F)C(=O)N1] |
| 46 | 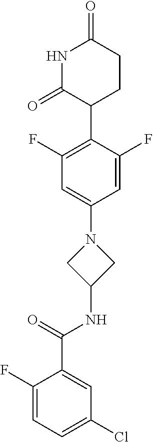 [SMILES: O=C1CCC(c2c(F)cc(N3CC(NC(=O)c4cc(Cl)ccc4F)C3)cc2F)C(=O)N1] |
| 47 | 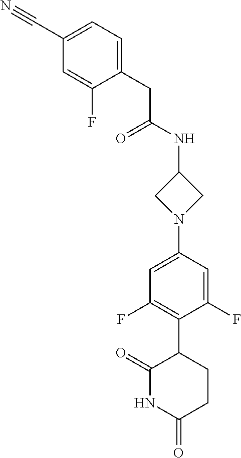 [SMILES: N#Cc1ccc(CC(=O)NC2CN(c3cc(F)c(C4CCC(=O)NC4=O)c(F)c3)C2)c(F)c1] |
| 48 | 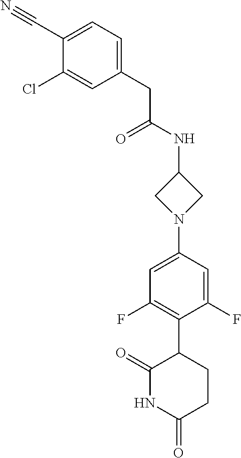 [SMILES: N#Cc1ccc(CC(=O)NC2CN(c3cc(F)c(C4CCC(=O)NC4=O)c(F)c3)C2)cc1Cl] |
| 49 | 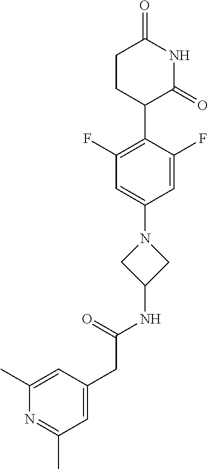 [SMILES: Cc1cc(CC(=O)NC2CN(c3cc(F)c(C4CCC(=O)NC4=O)c(F)c3)C2)cc(C)n1] |
| 50 | 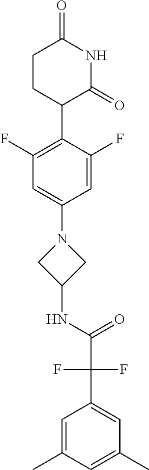 [SMILES: Cc1cc(C)cc(C(F)(F)C(=O)NC2CN(c3cc(F)c(C4CCC(=O)NC4=O)c(F)c3)C2)c1] |
| 51 | 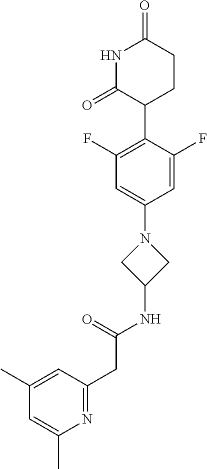 [SMILES: Cc1cc(C)nc(CC(=O)NC2CN(c3cc(F)c(C4CCC(=O)NC4=O)c(F)c3)C2)c1] |
| 52 | 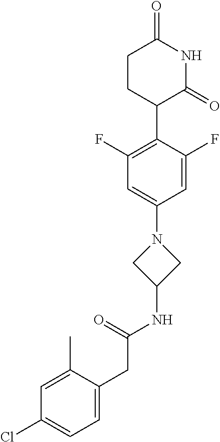 [SMILES: Cc1cc(Cl)ccc1CC(=O)NC1CN(c2cc(F)c(C3CCC(=O)NC3=O)c(F)c2)C1] |
| 53 | 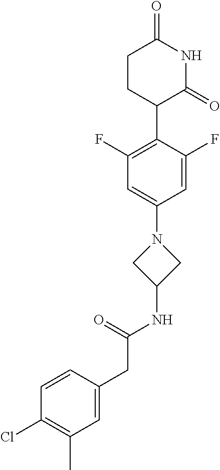 [SMILES: Cc1cc(CC(=O)NC2CN(c3cc(F)c(C4CCC(=O)NC4=O)c(F)c3)C2)ccc1Cl] |
| 54 | 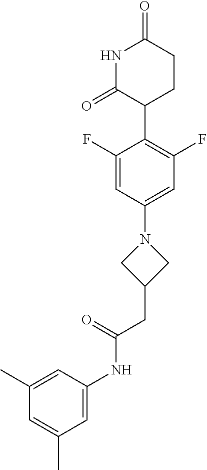 [SMILES: Cc1cc(C)cc(NC(=O)CC2CN(c3cc(F)c(C4CCC(=O)NC4=O)c(F)c3)C2)c1] |

### Pharmaceutical Compositions

In another embodiment, the present disclosure provides a pharmaceutical composition comprising a compound described herein, or pharmaceutically acceptable salt thereof, and a pharmaceutically acceptable excipient. In certain embodiments, the pharmaceutical composition comprises an effective amount of the compound. In certain embodiments, the pharmaceutical composition comprises a therapeutically effective amount of the compound.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Compositions for oral administration can take the form of bulk liquid solutions or suspensions, or bulk powders. In some embodiments, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component with the remainder being various vehicles or excipients and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component with the remainder being the injectable excipient and the like.

Transdermal compositions are typically formulated as a topical ointment or cream containing the active ingredient(s). When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope of the disclosure provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

### Methods of Treatment and Uses

Furthermore, the compounds and pharmaceutical compositions described herein are contemplated as useful in the treatment or prevention of disorders in subjects in need thereof. Compounds described herein, in one embodiment, are used to bind to and modulate the surface of cereblon to mediate ubiquitination of GSPT1 and subsequent degradation of said GSPT1 for the treatment of prevention of a disorder.

Accordingly, in one embodiment of the disclosure, a compound, or pharmaceutically acceptable salt thereof, or pharmaceutical composition described herein is administered to a subject to degrade GSPT1 in the subject. GSPT1 is a translational termination factor associated with cancers overexpressing one of the Myc family genes (c-Myc, N-Myc and L-Myc). The Myc transcription factors are some of the most frequently mutated, translocated and overexpressed oncogenes in human cancers. For example, around 10% of non-small cell lung cancer, or NSCLC, overexpress N-Myc and over 50% of small cell lung cancer, or SCLC, overexpress L-Myc. Myc-driven cancer cells are highly addicted to protein translation.

In one aspect of the disclosure, described herein is a method of treating or preventing a disorder in a subject in need thereof, comprising administering to the subject an effective amount of a compound, or pharmaceutically acceptable salt thereof, or pharmaceutical composition described herein.

In another aspect, described herein is a method of degrading GSPT1 in a subject suffering from a disorder, comprising administering to the subject a therapeutically effective amount of a compound described herein, or pharmaceutically acceptable salt thereof, or pharmaceutical composition described herein.

Exemplary disorders that can be treated or prevented by the methods of the present disclosure include but are not limited to, cancer of the bladder, bone, brain, breast, cervix, chest, colon, endometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands, neck, ovaries, pancreas, prostate, rectum, skin, stomach, testis, throat, or uterus. Other exemplary disorders include, but are not limited to, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer such as castration-resistant neuroendocrine prostate cancer (NEPC) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma; and blood bourne (liquid) or hematological cancers, including but not limited to leukemias, lymphomas, and myelomas, such as diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, ALK-positive large B-cell lymphoma, indolent lymphoma (for example, DLBCL, follicular lymphoma, or marginal zone lymphoma), acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, B-cell acute lymphoblastic leukemia, Non-Hodgkin's Lymphoma, Hodgkin's Lymphoma, and multiple myeloma (MM).

In another aspect of the disclosure, described herein is a method of treating cancer (e.g., a cancer described herein) in a subject in need thereof, comprising administering to the subject a therapeutically effective amount of a compound described herein, or pharmaceutically acceptable salt thereof, or pharmaceutical composition described herein.

In another aspect, described herein is a method of degrading GSPT1 in a subject suffering from cancer (e.g., a cancer described herein), comprising administering to the subject a therapeutically effective amount of a compound described herein, or pharmaceutically acceptable salt thereof, or pharmaceutical composition described herein.

In some embodiments, a cancer described herein is a Myc-driven cancer. In some embodiments, a cancer described herein is lung cancer, breast cancer, neuroendocrine cancer, or haematological cancer. In some embodiments, the lung cancer is non-small cell lung cancer (NSCLC). In some embodiments, the lung cancer is small-cell lung cancer (SCLC). In some embodiments, the hematological cancer is leukaemia or myeloma. In some embodiments, the myeloma is multiple myeloma.

In another aspect, described herein is a method of treating a solid tumor (e.g., a solid tumor described herein) in a subject in need thereof, comprising administering to the subject a therapeutically effective amount of a compound described herein, or pharmaceutically acceptable salt thereof, or pharmaceutical composition described herein.

In another aspect, described herein is a method of treating a liquid tumor (e.g., a liquid tumor described herein) in a subject in need thereof, comprising administering to the subject a therapeutically effective amount of a compound described herein, or pharmaceutically acceptable salt thereof, or pharmaceutical composition described herein. In some embodiments, the liquid tumor is that of a haematological cancer (e.g., a haematological cancer described herein).

In some embodiments, a method described herein comprises administering to the subject an additional therapeutic agent. In some embodiments, the additional therapeutic agent is an aminoglycoside or pharmaceutically acceptable salt thereof.

In another embodiment, provided herein is a method of preventing or treating a disease or disorder (e.g., a disease or disorder described herein) caused by or associated with one or more premature termination codons in a subject in need thereof, comprising administering to the subject a compound described herein or pharmaceutically acceptable salt thereof, or pharmaceutical composition thereof. In some embodiments, the method further comprises administering to the subject an aminoglycoside or pharmaceutically acceptable salt thereof. In some embodiments, the aminoglycoside is selected from geneticin, rhodostreptomycin, streptomycin, gentamicin, kanamycin A, tobramycin, neomycin B, neomycin C, framycetin, paromomycin, ribostamycin, amikacin, arbekacin, bekanamycin (kanamycin B), dibekacin, spectinomycin, hygromycin B, paromomycin sulfate, netilmicin, sisomicin, isepamicin, verdamicin, astromicin, neamine, ribostamycin, paromomycin, lividomycin, apramycin, and derivatives thereof. In some embodiments, the compound and aminoglycoside are administered in a simultaneous or sequential manner.

In another embodiment, described herein is a compound described herein or pharmaceutically acceptable salt thereof, or a pharmaceutical composition described herein, for use in degrading GSPT1 in a subject suffering from cancer, the use comprising administering a therapeutically effective amount of the compound or pharmaceutically acceptable salt thereof, or the pharmaceutical composition to the subject.

In another embodiment, provided herein is a compound described herein or pharmaceutically acceptable salt thereof, or a pharmaceutical composition described herein, for use in treating cancer in a subject in need thereof, the use comprising administering a therapeutically effective amount of the compound or pharmaceutically acceptable salt thereof, or the pharmaceutical composition to the subject.

In some embodiments, the cancer is lung cancer, breast cancer neuroendocrine cancer, or haematological cancer. In some embodiments, the haematological cancer is leukaemia or myeloma. In some embodiments, the myeloma is multiple myeloma. In some embodiments, the cancer is a Myc-driven cancer.

In another embodiment, provided herein is a compound described herein or pharmaceutically acceptable salt thereof, or a pharmaceutical composition described herein, for use in treating a solid tumor in a subject in need thereof, the use comprising administering a therapeutically effective amount of the compound or pharmaceutically acceptable salt thereof, or the pharmaceutical composition to the subject.

In another embodiment, provided herein is a compound described herein or pharmaceutically acceptable salt thereof, or a pharmaceutical composition described herein, for use in treating a liquid tumor in a subject in need thereof, the use comprising administering a therapeutically effective amount of the compound or pharmaceutically acceptable salt thereof, or the pharmaceutical composition to the subject.

In some embodiments, the use further comprises administering to the subject an additional therapeutic agent.

In an embodiment, provided herein is a compound described herein, or pharmaceutically acceptable salt thereof, or the pharmaceutical composition described herein, for use in preventing or treating a disease or disorder caused by or associated with one or more premature termination codons in a subject in need thereof, the use comprising administering to the subject the compound or pharmaceutically acceptable salt thereof, or the pharmaceutical composition. In some embodiments, the use further comprises administering to the subject an aminoglycoside or pharmaceutically acceptable salt thereof. In some embodiments, the aminoglycoside is selected from geneticin, rhodostreptomycin, streptomycin, gentamicin, kanamycin A, tobramycin, neomycin B, neomycin C, framycetin, paromomycin, ribostamycin, amikacin, arbekacin, bekanamycin (kanamycin B), dibekacin, spectinomycin, hygromycin B, paromomycin sulfate, netilmicin, sisomicin, isepamicin, verdamicin, astromicin, neamine, ribostamycin, paromomycin, lividomycin, apramycin, and derivatives thereof. In some embodiments, the compound and aminoglycoside are administered in a simultaneous or sequential manner.

### Myc-Driven Cancers

Described herein, in some embodiments, are cancers exhibiting increased expression of one or more of c-Myc, L-Myc, N-Myc, EIF4EBP1, and EIF4EBP2 as well as ones with increase phosphorylation of one or both of EIF4EBP1 and EIF4EBP2.

Myc-driven cancers refer to cancers where there is abnormal activation of Myc oncogene, either due to transcriptional overexpression (e.g., caused by gene amplification, translocation, alterations in upstream signaling pathways) and/or protein stabilization. A myc-driven cancer cell includes a cancer cell that has an increased expression or overexpression (and/or increased activity) of at least one myc transcription factor such as N-myc and/or L-myc and/or C-myc, or a surrogate marker thereof, relative to a control cell such as a normal (e.g., non-cancerous) cell of the same or corresponding cell type. The term “cancerous” when referring to a sample such as a cell or tissue, generally refers to any sample, such as cells or tissues that exhibit, or are predisposed to exhibiting, unregulated growth, including, for example, a neoplastic cell/tissue such as a premalignant cell/tissue or a cancer cell (e.g., carcinoma cell or sarcoma cell).

In some embodiments the Myc-driven cancer or tumor as defined herein refers to a blood borne tumor cancer, such as a hematological cancer, preferably a cancer of hematopoietic and lymphoid tissues and lymphatic system, such as blood cancer, bone marrow cancer, lymph node cancer, acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, non-Hodgkin's lymphomas and multiple myeloma (MM).

In some embodiments, the Myc-driven cancer or tumour is a solid tumor cancer, such as breast cancer, colorectal cancer, lung cancer, e.g. SCLC, NSCLC, neuroendocrine cancer, e.g., neuroendocrine prostate cancer (for example, NEPC (castration-resistant neuroendocrine prostate cancer)) and lung neuroendocrine tumors (Lu-NETs), liver cancer, stomach cancer, pancreatic cancer, gastric cancer, esophageal cancer, bladder cancer, skin cancer, brain cancer, cervical cancer, ovarian cancer, melanoma and head and neck cancer.

In some embodiments the Myc-driven cancer as used herein refers in particular to breast cancer and SCLC. In some embodiments the myc-driven cancer as used herein refers in particular to NSCLC. In some embodiments, the cancer is solid tumor cancer exhibiting amplification of the N-Myc gene and/or the L-Myc gene. In some embodiments the Myc-driven cancer as used herein refers to neuroendocrine cancer, for example, neuroendocrine prostate cancer (for example, NEPC (castration-resistant neuroendocrine prostate cancer)) and lung neuroendocrine tumors (Lu-NETs), acute myelogenous leukemia (AML), lymphoma, and multiple myeloma (MM).

### Solid and Liquid Cancers

The term “solid cancer” or “solid tumor” refers to disease of tissues or organs, such as to malignant, neoplastic, or cancerous solid tumors, i.e. sarcomas, carcinomas. The tissue structure of solid tumors includes interdependent tissue compartments and usually does not contain cysts or fluid areas. A solid cancer or solid tumor includes cancers of the bladder, bone, brain, breast, cervix, chest, colon, endometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands), neck, ovaries, pancreas, prostate, rectum, skin, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer (for example, NEPC (castration-resistant neuroendocrine prostate cancer)) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, cervical cancer, ovarian cancer, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In some embodiments, a solid cancer or solid tumor is a cancer of the breast, lung, stomach, colon, bladder, brain, pancreas, liver, head and neck, prostate, ovaries, upper aerodigestive tract and the like.

The term “blood borne cancer” or “blood borne tumor” (also typically referred to as “hematological cancer”) refers to cancer of the body's blood-forming and immune system—the bone marrow and lymphatic tissue. The tissue structure of blood-borne cancers or tumors includes an abnormal mass of cells that is fluid in nature. Such cancers include leukemias (malignant neoplasms of the blood-forming tissues), lymphomas (Non-Hodgkin's Lymphoma), Hodgkin's disease (Hodgkin's Lymphoma) and myeloma. In one embodiment, the myeloma is multiple myeloma (MM). In some embodiments, the leukemia is, for example, acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, or B-cell acute lymphoblastic leukemia. The leukemia can be relapsed, refractory or resistant to conventional therapy. In some embodiments, the lymphoma is, for example, diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, or ALK-positive large B-cell lymphoma. In one embodiment, the hematological cancer is indolent lymphoma including, for example, DLBCL, follicular lymphoma, or marginal zone lymphoma. In some embodiments blood-borne cancers or hematological cancers include acute lymphoblastic leukemia (ALL), chronic lymphocytic lymphoma (CLL), small lymphocytic lymphoma (SLL), acute myelogenous leukemia (AML), chronic myelogenous leukemia (CML), acute monocytic leukemia (AMoL), Hodgkin's lymphoma, non-Hodgkin's lymphomas and multiple myeloma (MM).

In particular embodiments, the compounds of the disclosure or pharmaceutically acceptable salts or stereoisomers thereof or a pharmaceutical composition thereof are used for the treatment of cancer associated with GSPT1, such as solid cancers including but not limited to cancers of the bladder, bone, brain, breast, cervix, chest, colon, endometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands), neck, ovaries, pancreas, prostate, rectum, skin, stomach, testis, throat, uterus, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer such as castration-resistant neuroendocrine prostate cancer (NEPC) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma; and blood bourne (liquid) or hematological cancers, including but not limited to leukemias, lymphomas, and myelomas, such as diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, ALK-positive large B-cell lymphoma, indolent lymphoma (for example, DLBCL, follicular lymphoma, or marginal zone lymphoma), acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, B-cell acute lymphoblastic leukemia, Non-Hodgkin's Lymphoma, Hodgkin's Lymphoma, and multiple myeloma (MM).

Such a use (or method of treatment) of a subject comprises administering to a subject in need of such treatment a therapeutically effective amount of a compound of the disclosure or pharmaceutically acceptable salts thereof or a pharmaceutical composition thereof by targeting cereblon.

Disclosed herein, in part, is a method of treating an Myc-driven cancer in a subject in need thereof, comprising administering the subject a therapeutically effective amount of a compound described herein or a composition as described herein.

In some embodiments, the Myc-driven cancer is an Myc-driven lung cancer.

In some embodiments, the Myc-driven cancer is characterized by high driven Myc tumor.

In some embodiments, the Myc-driven cancer is an Myc-driven small cell lung cancer.

In some embodiments, the Myc-driven small cell lung cancer is a high L-Myc small cell lung cancer.

In some embodiments, the Myc-driven cancer is an Myc-driven non-small cell lung cancer.

In some embodiments, the Myc-driven non-small cell lung cancer is a high N-Myc non-small cell lung cancer.

In some embodiments, the compound or the composition is administered to the subject via oral administration.

In another aspect, provided herein is a method of degrading GSPT1 in a subject suffering from cancer, comprising administering to the subject a therapeutically effective amount of a compound described herein or a composition described herein.

In some embodiments, the cancer is a Myc-driven cancer.

In some embodiments, the Myc-driven cancer is an Myc-driven lung cancer.

In some embodiments, the Myc-driven cancer is an Myc-driven small cell lung cancer.

In some embodiments, the Myc-driven small cell lung cancer is a high L-Myc small cell lung cancer.

In some embodiments, the Myc-driven cancer is an Myc-driven non-small cell lung cancer.

In some embodiments, the Myc-driven non-small cell lung cancer is a high N-Myc non-small cell lung cancer.

In some embodiments, the compound or the composition is administered to the subject via oral administration.

In another aspect, the disclosure is directed to a method of reducing the level of GSPT1 in a subject suffering from cancer, comprising administering the subject a therapeutically effective amount of a compound or a composition as described herein.

In some embodiments, the cancer is a Myc-driven cancer.

In some embodiments, the Myc-driven cancer is an Myc-driven lung cancer.

In some embodiments, the Myc-driven cancer is an Myc-driven small cell lung cancer.

In some embodiments, the Myc-driven small cell lung cancer is a high L-Myc small cell lung cancer.

In some embodiments, the Myc-driven cancer is an Myc-driven non-small cell lung cancer.

In some embodiments, the Myc-driven non-small cell lung cancer is a high N-Myc non-small cell lung cancer.

In some embodiments, the compound or the composition is administered to the subject via oral administration.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” as used herein refers to a radical of a straight-chain or branched saturated hydrocarbon group. In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₅) and the like. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

The term “alkenyl” as used herein refers to a radical of a straight-chain or branched hydrocarbon group having, one or more carbon-carbon double bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

The term “alkynyl” as used herein refers to a radical of a straight-chain or branched hydrocarbon group having one or more carbon-carbon triple bonds (e.g., 1, 2, 3, or 4 carbon-carbon triple bonds). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

The term “aminoglycoside” or “aminoglycoside of the disclosure” as used herein, refers to any aminoglycoside of the prior art and in particular to any aminoglycoside as defined in the description, as well as pharmaceutically acceptable salts and/or stereoisomers thereof.

The term “cycloalkyl” as used herein refers to a radical of a saturated or partially unsaturated cyclic hydrocarbon group having from 3 to 10 ring carbon atoms (“C₃₋₁₀ cycloalkyl”) and zero heteroatoms in the ring system. In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 3 to 6 ring carbon atoms (“C₃₋₆ cycloalkyl”). In some embodiments, a cycloalkyl group has 5 to 10 ring carbon atoms (“C₅₋₁₀ cycloalkyl”). Exemplary C₃₋₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ cycloalkyl groups include, without limitation, the aforementioned C₃₋₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ cycloalkyl groups include, without limitation, the aforementioned C₃₋₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”). “Cycloalkyl” also includes ring systems wherein the cycloalkyl ring, as defined above, is fused with one or more aryl or heteroaryl groups wherein the point of attachment is on the cycloalkyl ring or the one or more aryl or heteroaryl groups, and in such instances, the number of carbons continue to designate the number of carbons in the cycloalkyl ring system.

The term “heterocyclyl” as used herein refers to a radical of a saturated or partially unsaturated 3 to 10-membered ring system having ring carbon atoms and 1 to 4 ring heteroatoms, wherein each heteroatom is independently selected from nitrogen, oxygen, sulfur, boron, phosphorus, and silicon (“3 to 10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”). Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring or the one or more aryl or heteroaryl groups, and in such instances, the number of ring members continue to designate the number of ring members in the heterocyclyl ring system.

In some embodiments, a heterocyclyl group is a 5 to 10 membered saturated or partially unsaturated ring system having ring carbon atoms and 1 to 4 ring heteroatoms, wherein each heteroatom is independently selected from nitrogen, oxygen, sulfur, boron, phosphorus, and silicon (“5 to 10 membered heterocyclyl”). In some embodiments, a heterocyclyl group is a 5 to 8 membered saturated or partially unsaturated ring system having ring carbon atoms and 1 to 4 ring heteroatoms, wherein each heteroatom is independently selected from nitrogen, oxygen, and sulfur (“5 to 8 membered heterocyclyl”). In some embodiments, a heterocyclyl group is a 5 to 6 membered saturated or partially unsaturated ring system having ring carbon atoms and 1 to 4 ring heteroatoms, wherein each heteroatom is independently selected from nitrogen, oxygen, and sulfur (“5 to 6 membered heterocyclyl”). In some embodiments, the 5 to 6 membered heterocyclyl has 1 to 3 ring heteroatoms selected from nitrogen, oxygen, and sulfur. In some embodiments, the 5 to 6 membered heterocyclyl has 1 to 2 ring heteroatoms selected from nitrogen, oxygen, and sulfur. In some embodiments, the 5 to 6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

The term “aryl” as used herein refers to a radical of a monocyclic or polycyclic (e.g., bicyclic or tricyclic) 4n+2 aromatic ring system (e.g., having 6, 10, or 14 π electrons shared in a cyclic array) having 6 to 14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

The term “heteroaryl” as used herein refers to a radical of a 5 to 10 membered monocyclic or bicyclic 4n+2 aromatic ring system (e.g., having 6 or 10 π electrons shared in a cyclic array) having ring carbon atoms and 1 to 4 ring heteroatoms provided in the aromatic ring system, wherein each heteroatom is independently selected from nitrogen, oxygen and sulfur (“5 to 10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5 to 10 membered aromatic ring system having ring carbon atoms and 1 to 4 ring heteroatoms provided in the aromatic ring system, wherein each heteroatom is independently selected from nitrogen, oxygen, and sulfur (“5 to 10 membered heteroaryl”). In some embodiments, a heteroaryl group is a 5 to 8 membered aromatic ring system having ring carbon atoms and 1 to 4 ring heteroatoms provided in the aromatic ring system, wherein each heteroatom is independently selected from nitrogen, oxygen, and sulfur (“5 to 8 membered heteroaryl”). In some embodiments, a heteroaryl group is a monocyclic 5 to 6 membered aromatic ring system having ring carbon atoms and 1 to 4 ring heteroatoms provided in the aromatic ring system, wherein each heteroatom is independently selected from nitrogen, oxygen, and sulfur (“5 to 6 membered heteroaryl”). In some embodiments, the 5 to 6 membered heteroaryl has 1 to 3 ring heteroatoms selected from nitrogen, oxygen, and sulfur. In some embodiments, the 5 to 6 membered heteroaryl has 1 to 2 ring heteroatoms selected from nitrogen, oxygen, and sulfur. In some embodiments, the 5 to 6 membered heteroaryl has 1 ring heteroatom selected from nitrogen, oxygen, and sulfur. In some embodiments, a heteroaryl group is a monocyclic 5 membered aromatic ring system having ring carbon atoms and 1 to 4 ring heteroatoms provided in the aromatic ring system, wherein each heteroatom is independently selected from nitrogen, oxygen, and sulfur (“5-membered heteroaryl”). In some embodiments, a heteroaryl group is a monocyclic 6 membered aromatic ring system having ring carbon atoms and 1 to 4 ring heteroatoms provided in the aromatic ring system, wherein each heteroatom is independently selected from nitrogen, oxygen, and sulfur (“6-membered heteroaryl”).

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

The term “alkoxy” as used herein refers to the group —OR¹⁰⁰ where R¹⁰⁰ is alkyl, alkenyl, alkynyl, cycloalkyl, heterocyclyl, aryl, or heteroaryl. Exemplary alkoxy groups include methoxy, ethoxy, n-propoxy, isopropoxy, n-butoxy, tert-butoxy, sec-butoxy, n-pentoxy, n-hexoxy, and 1,2-dimethylbutoxy. Other exemplary alkoxy groups are lower alkoxy, i.e. with between 1 and 6 carbon atoms. In other examples, alkoxy groups have between 1 and 4 carbon atoms.

The term “cyano” as used herein refers to the radical —CN.

The term “halogen” as used herein refers to F, Cl, Br, or I.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition. In an alternative embodiment, the present disclosure contemplates administration of the compounds described herein as a prophylactic before a subject begins to suffer from the specified disease, disorder or condition.

In general, the “effective amount” of a compound as used herein refers to an amount sufficient to elicit the desired biological response. As will be appreciated by those of ordinary skill in this art, the effective amount of a compound of the present disclosure may vary depending on such factors as the desired biological endpoint, the pharmacokinetics of the compound, the disease being treated, the mode of administration, and the age, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Isomers, e.g., stereoisomers, can be isolated from mixtures by methods known to those skilled in the art, including chiral high pressure liquid chromatography (HPLC) and the formation of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The present disclosure additionally encompasses compounds described herein as individual isomers substantially free of other isomers, and alternatively, as mixtures of various isomers.

The compounds provided herein can be administered as the sole active agent, or they can be administered in combination with other active agents. In some embodiments, the present invention provides a combination of a compound of the present invention and another pharmacologically active agent. Administration in combination can proceed by any technique apparent to those of skill in the art including, for example, separate, sequential, concurrent, and alternating administration.

The present disclosure, in an alternative embodiment, also embraces isotopically labeled compounds which are identical to those recited herein, except that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

### EXAMPLES

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Abbreviations: Bn: benzyl; Boc: tert-butyloxycarbonyl; CRBN: cereblon; DMF: N,N-dimethylformamide; DMSO: dimethyl sulfoxide; eq: equivalents; EI: electron ionization; ESI: electrospray ionization; h: hours; HPLC: high-performance liquid chromatography; MS: mass spectrometry; MTBE: tert-butyl methyl ether; NMR: nuclear magnetic resonance; Py: pyridine; SEM: trimethylsilylethoxymethyl.

### Example 1: Synthesis of Intermediates, General Procedure A

### Synthesis of Intermediate IX:

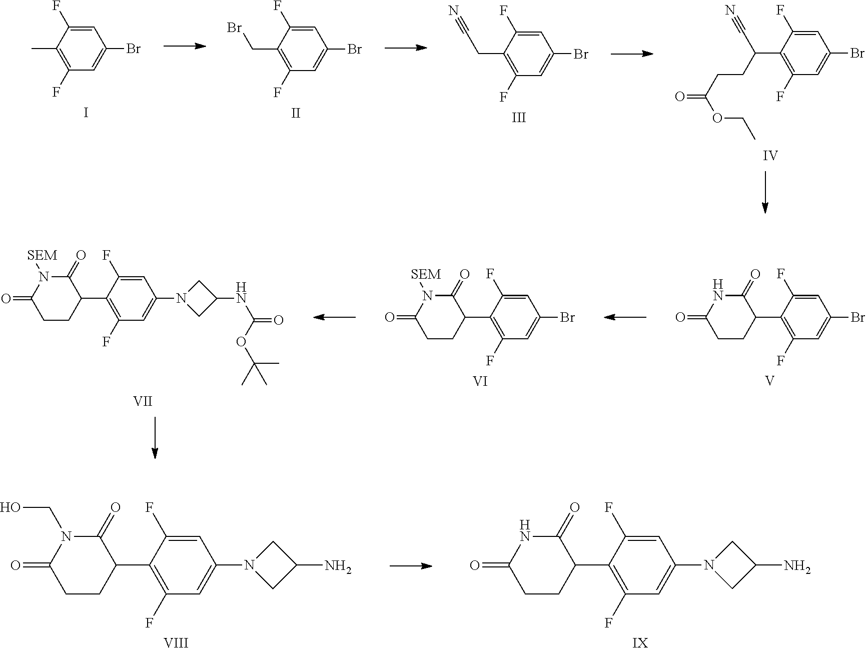

Intermediate II: To a solution of 5-bromo-1,3-difluoro-2-methylbenzene (19.3 g, 93.1 mmol, 1.00 eq) in tetrachloromethane (190 mL) was added N-bromosuccinimide (16.6 g, 93.1 mmol, 1.00 eq) and (E)-3,3′-(diazene-1,2-diyl)bis (2-methylpropanenitrile) (764 mg, 4.65 mmol, 0.0500 eq). The mixture was stirred at 80° C. for 2 h. The mixture was filtered and the filtrate was concentrated under reduced pressure to give a residue. The residue was purified by column chromatography (SiO₂, petroleum ether/ethyl acetate=1/0) and concentrated under reduced pressure to afford 5-bromo-2-(bromomethyl)-1,3-difluorobenzene II (21.2 g, 74.2 mmol, 80% yield) as a yellow oil. ¹H NMR (400 MHz, DMSO-d₆) δ=7.59-7.56 (m, 1H), 7.56-7.52 (m, 1H), 4.61 (s, 2H).

Intermediate III: To a solution of 5-bromo-2-(bromomethyl)-1,3-difluorobenzene II (21.2 g, 74.2 mmol, 1.00 eq) in ethanol (150 mL) and water (50 mL) was added potassium cyanide (3.81 mL, 89.0 mmol, 1.20 eq). The mixture was stirred at 60° C. for 2 h. The mixture was diluted with water (400 mL) and extracted with ethyl acetate (3×300 mL). The combined organic layers were dried over anhydrous sodium sulfate, filtered and concentrated under reduced pressure to give a residue. The residue was purified by column chromatography (SiO₂, petroleum ether/ethyl acetate=1/0 to 20/1) and concentrated under reduced pressure to afford 2-(4-bromo-2,6-difluorophenyl)acetonitrile III (12.2 g, 52.6 mmol, 71% yield) as a white solid. 1H NMR (400 MHz, DMSO-d6) δ=7.58 (s, 1H), 7.56 (s, 1H), 4.02 (s, 2H).

Intermediate IV: To a solution of 2-(4-bromo-2,6-difluorophenyl)acetonitrile III (12.2 g, 52.6 mmol, 1.00 eq), methyl acrylate (4.73 mL, 52.6 mmol, 1.00 eq) in tetrahydrofuran (120 mL) was added sodium methoxide (284.06 mg, 5.26 mmol, 0.100 eq) at 0° C. The mixture was stirred at 20° C. for 1 h. The mixture was quenched with saturated ammonium chloride aqueous solution (50 mL) and extracted with ethyl acetate (3×50 mL). The combined organic layers were washed with brine (2×300 mL), dried over anhydrous sodium sulfate, filtered and concentrated under reduced pressure to give a residue. The residue was purified by column chromatography (SiO₂, petroleum ether/ethyl acetate=1/0 to 10/1) and concentrated under reduced pressure to afford methyl 4-(4-bromo-2,6-difluorophenyl)-4-cyanobutanoate IV (13.5 g, 42.4 mmol, 81% yield) as a yellow oil. ¹H NMR (400 MHz, DMSO-d₆) δ=7.64-7.62 (m, 1H), 7.61-7.58 (m, 1H), 4.52 (t, J=7.8 Hz, 1H), 3.56 (s, 3H), 2.48-2.41 (m, 2H), 2.22 (qd, J=7.2, 14.0 Hz, 1H), 2.13-2.03 (m, 1H).

Intermediate V: To a solution of methyl 4-(4-bromo-2,6-difluorophenyl)-4-cyanobutanoate IV (13.5 g, 42.4 mmol, 1.00 eq) in acetic acid (135 mL) was added sulfuric acid (13.5 mL, 253 mmol, 5.97 eq). The mixture was stirred at 90° C. for 2 h. The mixture was poured into ice water (400 mL) and filtered. The filter cake was lyophilized to afford 3-(4-bromo-2,6-difluorophenyl)piperidine-2,6-dione V (15.0 g, crude) as a white solid. ¹H NMR (400 MHz, DMSO-d₆) δ=11.00 (s, 1H), 7.52 (s, 1H), 7.50 (s, 1H), 4.25 (dd, J=5.1, 12.7 Hz, 1H), 2.87-2.75 (m, 1H), 2.55 (br d, J=3.2 Hz, 1H), 2.12 (dq, J=3.9, 13.0 Hz, 1H), 2.06-1.97 (m, 1H).

Intermediate VI: To a solution of 3-(4-bromo-2,6-difluorophenyl)piperidine-2,6-dione (4.96 mL, 16.4 mmol, 1.00 eq) and 1,8-diazabicyclo[5.4.0]undec-7-ene (4.96 mL, 32.9 mmol, 2.00 eq) in tetrahydrofuran (50 mL) was added (2-trimethylethylsilylethoxy)methyl chloride (5.24 mL, 29.6 mmol, 1.80 eq) at 0° C. The reaction mixture was stirred at 20° C. for 12 h. 1,8-diazabicyclo[5.4.0]undec-7-ene (2.48 mL, 16.4 mmol, 1.00 eq) and (2-trimethylethylsilylethoxy)methyl chloride (2.91 mL, 16.4 mmol, 1.00 eq) was added at 0° C. and the reaction mixture was stirred at 20° C. for 12 h. The mixture was quenched with saturated sodium bicarbonate aqueous solution (80 mL) and extracted with ethyl acetate (3×70 mL). The combined organic layers were dried over anhydrous sodium sulfate, filtered and concentrated under reduced pressure to give a residue. The residue was purified by column chromatography (SiO₂, petroleum ether/ethyl acetate=1/0 to 3/1) and concentrated under reduced pressure to afford 3-(4-bromo-2,6-difluorophenyl)-1-((2-(trimethylsilyl)ethoxy)methyl) piperidine-2,6-dione VI (5.31 g, 12.2 mmol, 74% yield) as a yellow oil. ¹H NMR (400 MHz, DMSO-d₆) δ=7.55-7.52 (m, 1H), 7.52-7.48 (m, 1H), 5.10 (s, 2H), 4.41 (dd, J=5.1, 12.8 Hz, 1H), 3.55-3.49 (m, 2H), 3.04-2.92 (m, 1H), 2.78-2.70 (m, 1H), 2.15 (dq, J=4.0, 13.2 Hz, 1H), 2.08-1.99 (m, 1H), 0.86-0.79 (m, 2H), −0.01-−0.04 (m, 9H).

Intermediate VII: To a solution of 3-(4-bromo-2,6-difluorophenyl)-1-((2-(trimethylsilyl)ethoxy)methyl)piperidine-2,6-dione VI (3.00 g, 6.91 mmol, 1.00 eq), tert-butyl azetidin-3-yl carbamate (2.88 g, 13.8 mmol, 2.00 eq, hydrochloride) and cesium carbonate (6.75 g, 20.7 mmol, 3.00 eq) in toluene (30 mL) were added 4,5-bis(diphenylphos4,5-bis(diphenylphosphino)-9,9-dimethylxanthenephino)-9,9-dimethylxanthene (0.599 g, 1.04 mmol, 0.150 eq) and tris(dibenzylideneacetone)dipalladium(0) (0.316 g, 0.345 mmol, 0.0500 eq). The mixture was stirred at 110° C. for 12 h under nitrogen atmosphere. The mixture was filtered and concentrated under residue pressure to give a residue. The residue was purified by column chromatography (SiO₂, petroleum ether/ethyl acetate=1/0 to 5/1) to afford tert-butyl (1-(4-(2,6-dioxo-1-((2-(trimethylsilyl) ethoxy)methyl)piperidin-3-yl)-3,5-difluorophenyl)azetidin-3-yl) carbamate VII (2.70 g, 5.14 mmol, 74% yield) as a yellow oil. ¹H NMR (400 MHz, DMSO-d₆) δ=7.55 (br d, J=7.3 Hz, 1H), 6.14 (s, 1H), 6.12 (s, 1H), 5.12-5.05 (m, 2H), 4.45-4.34 (m, 1H), 4.18 (br dd, J=5.0, 12.6 Hz, 1H), 4.10-4.05 (m, 2H), 3.63-3.58 (m, 2H), 3.55-3.47 (m, 2H), 3.00-2.88 (m, 1H), 2.73-2.66 (m, 1H), 2.10 (dq, J=3.2, 12.9 Hz, 1H), 1.98-1.90 (m, 1H), 1.39 (s, 9H), 0.86-0.80 (m, 2H), −0.01-−0.05 (m, 9H).

Intermediate VIII: To a solution of tert-butyl (1-(4-(2,6-dioxo-1-((2-(trimethylsilyl)ethoxy)methyl)piperidin-3-yl)-3,5-difluorophenyl)azetidin-3-yl)carbamate VII (2.70 g, 5.14 mmol, 1.00 eq) in dichloromethane (20 mL) was added trifluoroacetic acid (10.0 mL, 15.4 g, 135 mmol, 26.3 eq). The mixture was stirred at 20° C. for 1 h. The mixture was concentrated under reduced pressure to afford 3-(4-(3-aminoazetidin-1-yl)-2,6-difluorophenyl)-1-(hydroxymethyl)piperidine-2,6-dione VIII (3.50 g, crude) as a yellow solid. MS (ESI) m/z. 326.1 [M+H]⁺.

Intermediate IX: To a solution of 3-(4-(3-aminoazetidin-1-yl)-2,6-difluorophenyl)-1-(hydroxymethyl)piperidine-2,6-dione (3.50 g, 10.8 mmol, 1.00 eq) in acetonitrile (40 mL) was added ammonium hydroxide (2.00 mL, 18.2 mmol, 35% purity, 1.69 eq). The mixture was stirred at 20° C. for 1 h. The mixture was concentrated under reduced pressure to give a residue. The residue was purified by reversed phase column chromatography (C18, 330 g; condition: water/acetonitrile=1/0 to 0/1, 0.1% formic acid) and lyophilized to afford 3-(4-(3-aminoazetidin-1-yl)-2,6-difluorophenyl)piperidine-2,6-dione IX (0.780 g, 2.29 mmol, 21% yield, formate) as a white solid and 3-(4-(3-aminoazetidin-1-yl)-2,6-difluorophenyl)piperidine-2,6-dione IX (0.650 g, 1.59 mmol, 15% yield, trifluoroacetate) as an off-white solid. ¹H NMR (400 MHz, DMSO-d₆) δ=10.87 (s, 1H), 8.33 (br s, 3H), 6.29 (s, 1H), 6.26 (s, 1H), 4.11 (br s, 3H), 4.05 (br dd, J=5.1, 12.6 Hz, 1H), 3.85-3.76 (m, 2H), 2.84-2.72 (m, 1H), 2.52 (br d, J=2.0 Hz, 1H), 2.14-2.02 (m, 1H), 1.98-1.89 (m, 1H).

### Synthesis of Intermediate XV:

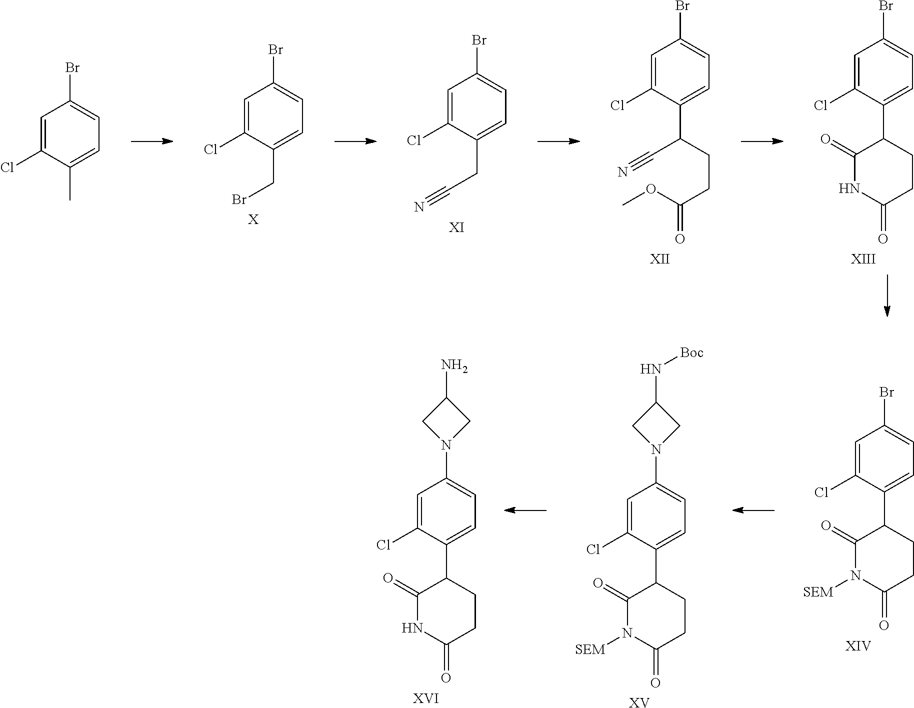

Intermediate X: To a solution of 4-bromo-2-chloro-1-methylbenzene (80.0 g, 389 mmol, 1.00 eq) in trichloromethane (600 mL) was added N-bromosuccinimide (76.4 g, 428 mmol, 1.10 eq) and benzoyl peroxide (9.44 g, 38.9 mmol, 0.100 eq). The mixture was stirred at 80° C. for 12 h under nitrogen. The mixture was extracted with water/ethyl acetate (800 mL/800 mL). The organic layer was collected and concentrated to give a residue. The residue was purified by column chromatography on silica gel (petroleum ether/ethyl acetate=1/0). The desired fraction was collected and concentrated to give 4-bromo-1-(bromomethyl)-2-chlorobenzene X (120 g, 359 mmol, 92% yield, 85% purity) as a yellow oil. ¹H NMR (400 MHz, DMSO-d₆) δ=7.81-7.70 (m, 1H), 7.56 (br s, 2H), 4.70 (s, 2H).

Intermediate XI: To a solution of 4-bromo-1-(bromomethyl)-2-chlorobenzene X (10.0 g, 35.2 mmol, 1.00 eq) in acetonitrile (50 mL) was added trimethylsilyl cyanide (13.2 mL, 105 mmol, 3.00 eq) and tetrabutylammonium fluoride (1M tetrahydrofuran) (1 M, 105 mL, 3.00 eq). The mixture was stirred at 25° C. for 1 h. The reaction mixture was quenched by addition water (100 mL), and then extracted with ethyl acetate (3×100 mL). The combined organic layers were dried over anhydrous sodium sulfate, filtered and concentrated under reduced pressure to give a residue. The residue was purified by column chromatography (SiO₂, petroleum ether/ethyl acetate=1/0 to 50/1) to give 2-(4-bromo-2-chlorophenyl)acetonitrile XI (6.16 g, 26.7 mmol, 76% yield) as a yellow oil. ¹H NMR (400 MHz, CDCl₃) δ=7.61 (d, J=2.0 Hz, 1H), 7.49-7.45 (m, 1H), 7.42-7.38 (m, 1H), 3.80 (s, 2H).

Intermediate XII: To a solution of 2-(4-bromo-2-chlorophenyl)acetonitrile XI (6.16 g, 26.7 mmol, 1.00 eq) and sodium methoxide (144 mg, 2.67 mmol, 0.100 eq) in tetrahydrofuran (50 mL) was added methyl acrylate (2.41 mL, 26.7 mmol, 1.00 eq) at 0° C. The mixture was stirred at 20° C. for 2 h. The reaction mixture was quenched by addition water (100 mL) at 0° C., and then extracted with ethyl acetate (3×100 mL). The combined organic layers were dried over anhydrous sodium sulfate, filtered and concentrated under reduced pressure to give a residue. The residue was purified by flash silica gel chromatography (ISCO®; 80 g SepaFlash® Silica Flash Column, Eluent of 0-100% ethyl acetate/petroleum ether gradient at 100 mL/min). Methyl 4-(4-bromo-2-chlorophenyl)-4-cyanobutanoate XII (6.16 g, 19.5 mmol, 73% yield) was obtained as a yellow solid. ¹H NMR (400 MHz, DMSO-d₆) δ=7.84 (d, J=2.1 Hz, 1H), 7.67 (dd, J=2.0, 8.4 Hz, 1H), 7.53 (d, J=8.4 Hz, 1H), 4.54 (dd, J=6.5, 8.4 Hz, 1H), 3.58 (s, 3H), 2.48-2.44 (m, 2H), 2.21-2.12 (m, 2H).

Intermediate XIII: A mixture of methyl 4-(4-bromo-2-chlorophenyl)-4-cyanobutanoate XII (6.15 g, 19.4 mmol, 1.00 eq), sulfuric acid (2.00 mL, 37.5 mmol, 1.93 eq) in acetic acid (20.0 mL, 349 mmol, 18.0 eq) was stirred at 90° C. for 2 h. The reaction mixture was quenched by addition saturated aqueous saturated sodium bicarbonate (150 mL) and extracted with ethyl acetate/dioxane (150 mL/100 mL). The combined organic layers were dried over anhydrous sodium sulfate, filtered and concentrated under reduced pressure to give 3-(4-bromo-2-chlorophenyl)piperidine-2,6-dione XIII (4.2 g, 13.8 mmol, 71% yield) as a yellow solid. ¹H NMR (400 MHz, DMSO-d₆) δ=10.93 (s, 1H), 7.74 (d, J=2.1 Hz, 1H), 7.55 (dd, J=2.1, 8.3 Hz, 1H), 7.32 (d, J=8.4 Hz, 1H), 4.21 (dd, J=5.0, 12.5 Hz, 1H), 2.84-2.71 (m, 1H), 2.59-2.52 (m, 1H), 2.30 (dq, J=4.3, 12.9 Hz, 1H), 1.97 (dtd, J=2.8, 5.2, 13.1 Hz, 1H).

Intermediate XIV: To a solution of 3-(4-bromo-2-chlorophenyl)piperidine-2,6-dione XIII (2.50 g, 8.26 mmol, 1.00 eq) in tetrahydrofuran (30 mL) and dimethyl formamide (3 mL) was added 1,8-diazabicyclo[5.4.0]-7-undecene (2.49 mL, 16.53 mmol, 2.00 eq) and (2-trimethylethylsilylethoxy)methyl chloride (2.63 mL, 14.9 mmol, 1.80 eq) at 0° C. The mixture was stirred at 25° C. for 1 h. The reaction mixture was quenched with water (50 mL), and then extracted with ethyl acetate (3×50. mL). The combined organic layers were washed with water (2×100 mL), dried over anhydrous sodium sulfate, filtered and concentrated under reduced pressure to give a residue. The residue was purified by column chromatography (SiO₂, petroleum ether/ethyl acetate=1/0 to 8/1) to give 3-(4-bromo-2-chlorophenyl)-1-((2-(trimethylsilyl)ethoxy) methyl)piperidine-2,6-dione XIV (5.73 g, 13.3 mmol, 80% yield) as a yellow oil. ¹H NMR (400 MHz, DMSO-d6) δ=7.76 (d, J=2.0 Hz, 1H), 7.56 (dd, J=2.1, 8.3 Hz, 1H), 7.31 (d, J=8.4 Hz, 1H), 5.08 (s, 2H), 4.36 (dd, J=4.9, 12.7 Hz, 1H), 3.56-3.50 (m, 2H), 3.00-2.87 (m, 1H), 2.78-2.69 (m, 1H), 2.38-2.27 (m, 1H), 2.04-1.94 (m, 1H), 0.85 (t, J=1.8 Hz, 1H), 0.83 (d, J=1.4 Hz, 1H), −0.03 (s, 9H).

Intermediate XV: A mixture of 3-(4-bromo-2-chlorophenyl)-1-((2-(trimethylsilyl)ethoxy)methyl)piperidine-2,6-dione XIV (5.70 g, 13.2 mmol, 1.00 eq), tert-butyl azetidin-3-ylcarbamate (5.50 g, 26.3 mmol, 2.00 eq, hydrochloric acid), cesium carbonate (12.9 g, 39.5 mmol, 3.00 eq) in dioxane (100 mL) was added tris(dibenzylideneacetone) dipalladium(0) (0.603 g, 0.658 mmol, 0.05 eq) and 4,5-bis(diphenylphosphino)-9,9-dimethylxanthene (1.14 g, 1.98 mmol, 0.150 eq). The mixture was stirred at 90° C. for 36 h under nitrogen atmosphere. The reaction mixture was diluted with water (100 mL) and extracted with ethyl acetate (3×100 mL). The combined organic layers were dried over anhydrous sodium sulfate, filtered and concentrated under reduced pressure to give a residue. The residue was purified by column chromatography (SiO₂, petroleum ether/ethyl acetate=0/1 to 5/1) to give tert-butyl (1-(3-chloro-4-(2,6-dioxo-1-((2-(trimethylsilyl)ethoxy)methyl) piperidin-3-yl)phenyl)azetidin-3-yl)carbamate XV (4.80 g, 8.97 mmol, 68% yield, 98% purity) as a yellow oil. ¹H NMR (400 MHz, DMSO-d6) δ=7.53 (br d, J=7.5 Hz, 1H), 7.06 (d, J=8.4 Hz, 1H), 6.47 (d, J=2.2 Hz, 1H), 6.37 (dd, J=2.3, 8.4 Hz, 1H), 5.08 (s, 2H), 4.45-4.35 (m, 1H), 4.16 (dd, J=5.0, 12.2 Hz, 1H), 4.10-4.05 (m, 2H), 3.60-3.50 (m, 4H), 2.89 (ddd, J=5.3, 12.6, 17.5 Hz, 1H), 2.73-2.65 (m, 1H), 2.23 (dq, J=4.2, 12.8 Hz, 1H), 1.96-1.90 (m, 1H), 1.39 (s, 9H), 0.85-0.81 (m, 2H), 0.01-−0.05 (m, 9H). MS (ESI) m/z 524.1 [M+H]⁺.

Intermediate XVI: To a solution of tert-butyl (1-(3-chloro-4-(2,6-dioxo-1-((2-(trimethylsilyl)ethoxy)methyl)piperidin-3-yl) phenyl)azetidin-3-yl)carbamate XV (4.80 g, 9.16 mmol, 1.00 eq) in dichloromethane (50 mL) was added trifluoroacetic acid (10.0 mL, 135 mmol, 14.7 eq). The mixture was stirred at 25° C. for 1 h. The reaction mixture was concentrated under reduced pressure to give 3-(4-(3-aminoazetidin-1-yl)-2-chlorophenyl)-1-(hydroxymethyl)piperidine-2,6-dione (3.80 g, crude) as a yellow oil. The crude product was dissolved in acetonitrile (50 mL)/ammonium hydroxide (5.24 mL). The mixture was stirred at 25° C. for 10 min. The reaction mixture was concentrated under reduced pressure to give a residue. The residue was purified by reverse phase HPLC (column: spherical C18, 20-45 μm, 100 Å, SW 120, mobile phase: [water (0.1% formic acid)-acetonitrile) to give 3-(4-(3-aminoazetidin-1-yl)-2-chlorophenyl)piperidine-2,6-dione XV (1.02 g, 3.16 mmol, 27% yield, 91% purity) as an off-white solid. MS (ESI) m/z 294.0 [M+H]⁺.

### Example 2. Synthesis of Compound 1

Step 1. To a solution of dimethyl malonate (739 μL, 6.43 mmol, 1.00 eq) in tetrahydrofuran (10 mL) was added sodium hydride (257 mg, 6.43 mmol, 60% purity, 1.00 eq) at 0° C. The mixture was stirred at 0° C. for 30 min. Then 3-chloro-4-fluorobenzonitrile (1.00 g, 6.43 mmol, 1.00 eq) was added into the mixture and it was stirred at 80° C. for 11.5 h. The mixture was quenched by adding saturated ammonium chloride solution (25 mL) at 0° C. (pH=6-7). The mixture was extracted with ethyl acetate (3×80 mL). The organic layers were washed with brine (60 mL), dried over anhydrous sodium sulfate, filtered and concentrated to give a residue. The residue was purified by silica gel chromatography (SiO₂, petroleum ether/ethyl acetate=1/0 to 5/1) to afford dimethyl 2-(2-chloro-4-cyanophenyl)malonate (700 mg, 2.62 mmol, 41% yield) as a white solid. ¹H NMR (400 MHz, DMSO-d₆) δ=8.15 (d, J=1.6 Hz, 1H), 7.88 (dd, J=1.6, 8.1 Hz, 1H), 7.62 (d, J=8.1 Hz, 1H), 5.38 (s, 1H), 3.73 (s, 6H).

Step 2. A mixture of dimethyl 2-(2-chloro-4-cyanophenyl)malonate (300 mg, 1.12 mmol, 1.00 eq) in dimethyl sulfoxide (1 mL) and water (1 mL) was added lithium chloride (68.9 μL, 3.36 mmol, 3.00 eq). The mixture was stirred at 110° C. for 12 h. The mixture was added water (30 mL) and extracted with ethyl acetate (3×50 mL). The combined organic phases were washed with brine (40 mL), dried with anhydrous sodium sulfate, filtered and concentrated under reduced pressure to afford methyl 2-(2-chloro-4-cyanophenyl)acetate (130 mg, 0.620 mmol, 55% yield) as a white solid. ¹H NMR (400 MHz, DMSO-d₆) δ=8.06 (s, 1H), 7.82 (dd, J=1.0, 7.9 Hz, 1H), 7.64 (d, J=7.9 Hz, 1H), 3.94 (s, 2H), 3.63 (s, 3H).

Step 3. To a solution of methyl 2-(2-chloro-4-cyanophenyl)acetate (100 mg, 477 μmol, 1.00 eq) in methanol (1.20 mL) and water (0.400 mL) was added lithium hydroxide monohydrate (22.9 mg, 954 μmol, 2.00 eq). The mixture was stirred at 25° C. for 2 h. The mixture was extracted with ethyl acetate (40 mL). The aqueous phases were added hydrochloric acid (1M, 10 mL) and extracted with ethyl acetate (3×40 mL). The combined organic phases were washed with brine (20 mL), dried with anhydrous sodium sulfate, filtered and concentrated under reduced pressure to afford 2-(2-chloro-4-cyanophenyl)acetic acid (90.0 mg, 460 μmol, 96% yield) as a yellow solid. ¹H NMR (400 MHz, DMSO-d₆) δ=12.96-12.48 (m, 1H), 8.05 (d, J=1.5 Hz, 1H), 7.80 (dd, J=1.5, 7.9 Hz, 1H), 7.62 (d, J=7.9 Hz, 1H), 3.83 (s, 2H).

Step 4. To a solution of 3-(4-(3-aminoazetidin-1-yl)-2,6-difluorophenyl)piperidine-2,6-dione IX (80.0 mg, 234 μmol, 1.00 eq, formate), 2-(2-chloro-4-cyanophenyl)acetic acid (45.9 mg, 234 μmol, 1.00 eq), N,N-diisopropylethylamine (122 μL, 703 μmol, 3.00 eq) and 1-(3-dimethylaminopropyl)-3-ethylcarbodiimide hydrochlorid (58.4 mg, 305 μmol, 1.30 eq) in dimethylformamide (1 mL) was added 1-hydroxybenzotriazole (31.7 mg, 234 μmol, 1.00 eq) at 0° C. The mixture was stirred at 25° C. for 1 h. The mixture was filtered. The filtrate was purified by Prep-HPLC (column: Phenomenex Synergi C18 150*25 mm*10 μm; mobile phase: [water (formic acid)-acetonitrile]; B %: 33%-55%, 11 min) and lyophilized to afford 2-(2-chloro-4-cyanophenyl)-N-(1-(4-(2,6-dioxopiperidin-3-yl)-3,5-difluorophenyl)azetidin-3-yl)acetamide #1 (61.29 mg, 128 μmol, 55% yield, 99% purity) as a white solid. ¹H NMR (400 MHz, DMSO-d₆) δ=10.86 (s, 1H), 8.85 (d, J=6.9 Hz, 1H), 8.03 (d, J=1.6 Hz, 1H), 7.79 (dd, J=1.7, 8.0 Hz, 1H), 7.58 (d, J=8.0 Hz, 1H), 6.18 (d, J=11.0 Hz, 2H), 4.61-4.52 (m, 1H), 4.12 (t, J=7.7 Hz, 2H), 4.04 (br dd, J=5.3, 12.5 Hz, 1H), 3.72 (s, 2H), 3.66 (dd, J=5.5, 7.8 Hz, 2H), 2.84-2.72 (m, 1H), 2.52 (br s, 1H), 2.14-2.01 (m, 1H), 1.98-1.90 (m, 1H). MS (ESI) m/z 472.8 [M+H]⁺.

### Example 3. Synthesis of Compound 2

To a solution of 3-(4-(3-aminoazetidin-1-yl)-2,6-difluorophenyl)piperidine-2,6-dione IX (70.0 mg, 205 μmol, 1.00 eq, formate), 2-cyclobutylacetic acid (23.41 mg, 205 μmol, 1.00 eq), N,N-diisopropylethylamine (107 μL, 615 μmol, 3.00 eq) and 1-(3-dimethylaminopropyl)-3-ethylcarbodiimide hydrochloride (51.1 mg, 267 μmol, 1.30 eq) in dimethylformamide (1 mL) was added 1-hydroxybenzotriazole (27.7 mg, 205 μmol, 1.00 eq) at 0° C. The mixture was stirred at 25° C. for 1 h. The mixture was filtered. The filtrate was purified by Prep-HPLC (column: Phenomenex Synergi C18 150*25 mm*10 μm; mobile phase: [water (formic acid)-acetonitrile]; B %: 33%-53%, 10 min) and lyophilized to give a residue. The residue was purified by Prep-HPLC (column: Phenomenex Synergi C18 150*25 mm*10 μm; mobile phase: [water (formic acid)-acetonitrile]; B %: 30%-60%, 10 min) and lyophilized to afford 2-cyclobutyl-N-(1-(4-(2,6-dioxopiperidin-3-yl)-3,5-difluorophenyl) azetidin-3-yl)acetamide #2 (25.87 mg, 65.4 μmol, 32% yield, 99% purity) as a white solid. ¹H NMR (400 MHz, DMSO-d₆) δ=10.86 (s, 1H), 8.48-8.40 (m, 1H), 6.16 (d, J=11.1 Hz, 2H), 4.58-4.49 (m, 1H), 4.09 (t, J=7.8 Hz, 2H), 4.03 (br dd, J=4.9, 12.5 Hz, 1H), 3.59 (dd, J=5.7, 7.7 Hz, 2H), 2.82-2.73 (m, 1H), 2.58 (br d, J=7.9 Hz, 2H), 2.19 (d, J=7.6 Hz, 2H), 2.12-1.92 (m, 4H), 1.86-1.72 (m, 2H), 1.71-1.58 (m, 2H). MS (ESI) m/z 392.2 [M+H]⁺.

### Example 4. Synthesis of Compound 3

Step 1. To a solution of 2-(1-(tert-butoxycarbonyl)azetidin-3-yl)acetic acid (2.00 g, 9.29 mmol, 1.00 eq) in dichloromethane (20 mL) were added 2-(3H-[1,2,3]triazolo[4,5-b]pyridin-3-yl)-1,1,3,3-tetramethylisouronium hexafluorophosphate(V) (5.30 g, 13.9 mmol, 1.50 eq) and N,N-diisopropylethylamine (4.86 mL, 27.8 mmol, 3.00 eq). The mixture was stirred at 25° C. for 1 h. 4-chloro-3-methylaniline (1.58 g, 11.1 mmol, 1.20 eq) was added into this mixture. The mixture was stirred at 25° C. for 2 h. The mixture was concentrated under reduced pressure to give a residue. The residue was diluted with water (50 mL), extracted with ethyl acetate (3×50 mL), dried over anhydrous sodium sulfate, filtered and concentrated under reduced pressure to give a residue. The residue was purified by column chromatography (SiO2, petroleum ether/ethyl acetate=2/1 to 1/1) to afford tert-butyl 3-(2-((4-chloro-3-methylphenyl)amino)-2-oxoethyl)azetidine-1-carboxylate (3.00 g, 8.85 mmol, 95% yield) as a light yellow solid. ¹H NMR (400 MHz, DMSO-d6) δ=10.01 (s, 1H), 7.57 (d, J=2.3 Hz, 1H), 7.40 (dd, J=2.4, 8.7 Hz, 1H), 7.30 (d, J=8.7 Hz, 1H), 3.95 (br s, 2H), 3.57 (br s, 2H), 2.88-2.79 (m, 1H), 2.63 (d, J=7.7 Hz, 2H), 2.28 (s, 3H), 1.37 (s, 9H).

Step 2. To a solution of tert-butyl 3-(2-((4-chloro-3-methylphenyl)amino)-2-oxoethyl)azetidine-1-carboxylate (100 mg, 295 μmol, 1.00 eq) in dichloromethane (1 mL) was added trifluoroacetic acid (0.200 mL). The mixture was stirred at 25° C. for 1 h. The mixture was concentrated under reduced pressure to afford 2-(azetidin-3-yl)-N-(4-chloro-3-methylphenyl)acetamide (600 mg, crude) as yellow oil. MS (ESI) m/z 239.2 [M+H]⁺.

Step 3. To a solution of 3-(4-bromo-2,6-difluorophenyl)piperidine-2,6-dione V (60.0 mg, 197 μmol, 1.00 eq) in dioxane (2 mL) were added 2-(azetidin-3-yl)-N-(4-chloro-3-methylphenyl)acetamide (189 mg, 295 μmol, 55% purity, 1.50 eq), cesium carbonate (32.0 mg, 986 μmol, 5.00 eq), palladium acetate (4.43 mg, 19.7 μmol, 0.100 eq) and 4,5-bis(diphenylphosphino)-9,9-dimethylxanthene (11.4 mg, 19.7 μmol, 0.100 eq). The mixture was stirred at 100° C. for 12 h under nitrogen atmosphere. The mixture was filtered and the filtrate was concentrated under reduced pressure to give a residue. The residue was purified by reverse phase column chromatography (C18, 330 g; condition: water/acetonitrile=1/0 to 0/1, 0.1% formic acid) and lyophilized to give a residue. The residue was purified by Prep-TLC (petroleum ether: ethyl acetate=1:1) and concentrated under reduced pressure to give a crude product. The crude product was purified by Prep-HPLC (column: Waters Xbridge 150*25 mm*5 μm; mobile phase: [water-acetonitrile]; B %: 47%-77%, 10 min) and lyophilized to afford N-(4-chloro-3-methylphenyl)-2-(1-(4-(2,6-dioxopiperidin-3-yl)-3,5-difluorophenyl)azetidin-3-yl)acetamide #3 (20.44 mg, 43.3 μmol, 7% yield, 98% purity) as a white solid. ¹H NMR (400 MHz, DMSO-d₆) δ=10.85 (br s, 1H), 10.05 (s, 1H), 7.59 (d, J=2.3 Hz, 1H), 7.42 (dd, J=2.4, 8.7 Hz, 1H), 7.31 (d, J=8.7 Hz, 1H), 6.13 (s, 1H), 6.10 (s, 1H), 4.04 (br d, J=5.3 Hz, 1H), 4.00-3.95 (m, 2H), 3.56 (br t, J=6.2 Hz, 2H), 3.08-2.98 (m, 1H), 2.82-2.74 (m, 1H), 2.73-2.68 (m, 2H), 2.52 (d, J=1.9 Hz, 1H), 2.28 (s, 3H), 2.13-2.01 (m, 1H), 1.97-1.89 (m, 1H). MS (ESI) m/z 462.1 [M+H]⁺.

### Example 5. Synthesis of Compound 4

To a solution of 3-(4-(3-aminoazetidin-1-yl)-2,6-difluorophenyl)piperidine-2,6-dione IX (60.0 mg, 176 μmol, 1.00 eq, formate), 2-(5-chloro-2,4-difluorophenyl)acetic acid (36.3 mg, 176 μmol, 1.00 eq), N,N-diisopropylethylamine (91.9 μL, 527 umol, 3.00 eq) and 1-(3-dimethylaminopropyl)-3-ethylcarbodiimide hydrochlorid (43.8 mg, 229 μmol, 1.30 eq) in dimethylformamide (1 mL) was added 1-hydroxybenzotriazole (23.8 mg, 176 μmol, 1.00 eq) at 0° C. The mixture was stirred at 25° C. for 2 h. The filtrate was purified by Prep-HPLC (column: Phenomenex Synergi C18 150*25 mm*10 μm; mobile phase: [water (formic acid)-acetonitrile]; B %: 37%-67%, 10 min) and lyophilized to afford 2-(5-chloro-2,4-difluorophenyl)-N-(1-(4-(2,6-dioxopiperidin-3-yl)-3,5-difluorophenyl)azetidin-3-yl)acetamide #4 (24.09 mg, 44.6 μmol, 25% yield, 98% purity, formate) as a grey solid. ¹H NMR (400 MHz, DMSO-d6) δ=10.87 (s, 1H), 8.80 (d, J=7.0 Hz, 1H), 7.61 (t, J=8.0 Hz, 1H), 7.49 (t, J=9.5 Hz, 1H), 6.18 (d, J=11.0 Hz, 2H), 4.60-4.51 (m, 1H), 4.11 (t, J=7.8 Hz, 2H), 4.04 (br dd, J=5.1, 12.7 Hz, 1H), 3.64 (dd, J=6.0, 7.3 Hz, 2H), 3.52 (s, 2H), 2.84-2.71 (m, 1H), 2.55-2.52 (m, 1H), 2.13-2.03 (m, 1H), 1.98-1.90 (m, 1H). MS (ESI) m/z 484.0 [M+H]⁺.

### Example 6. Synthesis of Compound 5

Step 1. A mixture of 1-chloro-4-fluoro-2-methyl-5-nitro-benzene (2.00 g, 10.5 mmol, 1.00 eq), iron power (2.95 g, 52.7 mmol, 5.00 eq) and ammonium chloride (2.82 g, 52.7 mmol, 5.00 eq) in methanol (10 mL) and water (5 mL). The reaction mixture was stirred at 80° C. for 2 h. The reaction mixture was filtered and concentrated to give a residue. The residue was diluted with saturated sodium bicarbonate (100 mL) and extracted with ethyl acetate (3×50.0 mL). The combined organic layers were washed with brine (20 mL), dried over anhydrous sodium sulfate, filtered and concentrated under reduced pressure to afford 5-chloro-2-fluoro-4-methylaniline (1.60 g, 10.0 mmol, 95% yield) as a yellow solid. ¹H NMR (400 MHz, DMSO-d₆) δ=6.98 (d, J=12.0 Hz, 1H), 6.80 (d, J=8.4 Hz, 1H), 5.19 (s, 2H), 2.15 (s, 3H). MS (ESI) m/z 160.1 [M+H]⁺.

Step 2. To a mixture of 2-(1-(tert-butoxycarbonyl)azetidin-3-yl)acetic acid (500 mg, 2.32 mmol, 1.00 eq) and N,N-diisopropylethylamine (1.21 mL, 6.97 mmol, 3.00 eq) and O-(7-azabenzotriazol-1-yl)-N,N,N,N-tetramethyluroniumhexafluorophosphate (1.06 g, 2.79 mmol, 1.20 eq) in dichloromethane (10 mL) was added 5-chloro-2-fluoro-4-methylaniline (407 mg, 2.55 mmol, 1.10 eq). The reaction mixture was stirred at 20° C. for 1 h. The mixture was concentrated in vacuum to give a residue, which was purified by silica gel chromatography (Petroleum ether/Ethyl acetate=10/1 to 1/1) to give tert-butyl 3-(2-((5-chloro-2-fluoro-4-methylphenyl)amino)-2-oxoethyl)azetidine-1-carboxylate (600 mg, 1.68 mmol, 72% yield) as a yellow solid. ¹H NMR (400 MHz, DMSO-d₆) δ=9.87 (s, 1H), 7.99 (d, J=7.2 Hz, 1H), 7.32 (d, J=11.6 Hz, 1H), 3.95 (br s, 2H), 3.57 (br s, 2H), 2.91-2.79 (m, 1H), 2.71 (d, J=7.6 Hz, 2H), 2.29 (s, 3H), 1.38 (s, 9H). MS (ESI) m/z 301.1 [M-56+1]⁺.

Step 3. To a mixture of tert-butyl 3-(2-((5-chloro-2-fluoro-4-methylphenyl)amino)-2-oxoethyl)azetidine-1-carboxylate (100 mg, 280 μmol, 1.00 eq) in dichloromethane (3 mL) was added trifluoroacetic acid (1 mL). The reaction mixture was stirred at 25° C. for 2 h. The reaction mixture was concentrated in vacuum to give 2-(azetidin-3-yl)-N-(5-chloro-2-fluoro-4-methylphenyl)acetamide (70.0 mg, crude) as a yellow oil and was used to the nect step without purification. MS (ESI) m/z 257.1 [M+H]⁺.

Step 4. A mixture of 3-(4-bromo-2,6-difluorophenyl)piperidine-2,6-dione V (55.2 mg, 181 μmol, 1.00 eq), 2-(azetidin-3-yl)-N-(5-chloro-2-fluoro-4-methylphenyl)acetamide (70.0 mg, 272 μmol, 1.50 eq), cesium carbonate (177 mg, 545 μmol, 3.00 eq) and tris(dibenzylideneacetone)dipalladium(0) (16.6 mg, 18.1 μmol, 0.100 eq) in dioxane (2 mL) was added 4,5-bis(diphenylphosphino)-9,9-dimethylxanthene (10.5 mg, 18.1 μmol, 0.100 eq) under nitrogen atmosphere. The reaction mixture was stirred at 100° C. for 12 h. The reaction mixture was filtered and concentrated to give a residue. The residue was purified by Prep-NPLC (column: Welch Ultimate XB-CN 250*50*10 μm; mobile phase: [hexane-ethyl alcohol]; B %: 15%-55%, 15 min) followed by Prep-HPLC (column: Phenomenex Luna C18 150*25 mm*10 μm; mobile phase: [water (formic acid)-acetonitrile]; B %: 41%-71%, 10 min) and lyophilized to give N-(5-chloro-2-fluoro-4-methylphenyl)-2-(1-(4-(2,6-dioxopiperidin-3-yl)-3,5-difluorophenyl)azetidin-3-yl)acetamide #5 (4.49 mg, 9.38 μmol, 5% yield, 99% purity, formic acid) as a yellow solid. ¹H NMR (400 MHz, DMSO-d₆) δ=10.86 (s, 1H), 9.89 (s, 1H), 8.01 (d, J=7.2 Hz, 1H), 7.32 (d, J=11.6 Hz, 1H), 6.12 (d, J=11.2 Hz, 2H), 4.06-4.01 (m, 1H), 4.01-3.95 (m, 2H), 3.55 (br t, J=6.6 Hz, 2H), 3.03 (td, J=6.4, 12.8 Hz, 1H), 2.83-2.72 (m, 3H), 2.48-2.45 (m, 1H), 2.28 (s, 3H), 2.14-2.01 (m, 1H), 1.98-1.89 (m, 1H). MS (ESI) m/z 480.3 [M+H]⁺.

### Example 7. Synthesis of Compound 6

Step 1. To a mixture of 2-fluoro-5-nitrophenol (10.0 g, 63.7 mmol, 1.00 eq) and sodium 2-chloro-2,2-difluoroacetate (24.3 g, 159 mmol, 2.50 eq) in dimethylformamide (100 mL) was added cesium carbonate (41.5 g, 127 mmol, 2.00 eq) in one portion. The mixture was stirred at 100° C. for 2 h. The mixture was diluted with water (200 mL) and extracted with ethyl acetate (3×200 mL). The combined organic layer was washed with brine (100 mL), dried over sodium sulfate, filtered and concentrated in vacuum. The residue was purified by silica gel chromatography (petroleum ether/ethyl acetate=1/0 to 10/1) to give 2-(difluoromethoxy)-1-fluoro-4-nitrobenzene (2.90 g, 14.0 mmol, 22% yield) as a yellow oil. ¹H NMR (400 MHz, DMSO-d₆) δ=8.33-8.16 (m, 2H), 7.72 (t, J=9.5 Hz, 1H), 7.44 (t, J=72.4 Hz, 1H).

Step 2. To a mixture of 2-(difluoromethoxy)-1-fluoro-4-nitrobenzene (2.90 g, 14.0 mmol, 1.00 eq) and ammonium chloride (3.75 g, 70.0 mmol, 5.00 eq) in methanol (40 mL) and water (20 mL) was added iron powder (2.35 g, 42 mmol, 3.00 eq) in portion. The mixture was stirred at 80° C. for 2 h. The mixture was diluted with water (80 mL) and extracted with ethyl acetate (3×80 mL). The combined organic layer was washed with brine (50 mL), dried over sodium sulfate, filtered and concentrated in vacuum to give 3-(difluoromethoxy)-4-fluoroaniline (2.10 g, 11.9 mmol, 85% yield) as a yellow oil. ¹H NMR (400 MHz, DMSO-d₆) δ=7.10 (t, J=73.6 Hz, 1H), 6.99 (dd, J=8.9, 10.8 Hz, 1H), 6.49 (dd, J=2.6, 6.8 Hz, 1H), 6.40 (td, J=3.2, 8.8 Hz, 1H), 5.21 (br s, 2H).

Step 3. To a mixture of 3-(difluoromethoxy)-4-fluoroaniline (2.00 g, 11.3 mmol, 1.00 eq), 2-(1-(tert-butoxycarbonyl) azetidin-3-yl)acetic acid (2.92 g, 13.6 mmol, 1.20 eq) and N,N-diisopropylethylamine (5.90 mL, 33.9 mmol, 3.00 eq) in dichloromethane (20 mL) was added 0-(7-azabenzotriazol-1-yl)-N,N,N′,N′-tetramethyluronium hexafluorophosphate (6.44 g, 16.9 mmol, 1.50 eq) in one portion. The mixture was stirred at 25° C. for 2 h. The mixture was diluted with water (150 mL) and extracted with ethyl acetate (3×80 mL). The combined organic layer was washed with brine (60 mL), dried over sodium sulfate, filtered and concentrated in vacuum. The residue was purified by silica gel chromatography (petroleum ether/ethyl acetate=3/1) and concentrated in vacuum. The residue was purified by reverse phase chromatography (column: spherical C18, 20-45 μm, 100 Å, SW 120, mobile phase: [water (0.1% formic acid)-acetonitrile) and lyophilized to give tert-butyl 3-(2-((3-(difluoromethoxy)-4-fluorophenyl)amino)-2-oxoethyl)azetidine-1-carboxylate (3.50 g, 9.35 mmol, 83% yield) as a yellow solid. ¹H NMR (400 MHz, DMSO-d₆) δ=10.17 (s, 1H), 7.75 (dd, J=1.9, 7.3 Hz, 1H), 7.35-7.29 (m, 2H), 7.19 (t, J=72.8 Hz, 1H), 4.00-3.89 (m, 2H), 3.57 (br s, 2H), 2.91-2.79 (m, 1H), 2.64 (d, J=7.8 Hz, 2H), 1.37 (s, 9H).

Step 4. A mixture of tert-butyl 3-(2-((3-(difluoromethoxy)-4-fluorophenyl)amino)-2-oxoethyl)azetidine-1-carboxylate (1.00 g, 2.67 mmol, 1.00 eq) in trifluoroacetic acid (2 mL) and dichloromethane (8.00 mL) was stirred at 25° C. for 1 h. The mixture was concentrated in vacuum. The residue was added water (20 mL) and lyophilized to give 2-(azetidin-3-yl)-N-(3-(difluoromethoxy)-4-fluorophenyl)acetamide (730 mg, crude) as a yellow oil. MS (ESI) m/z 275.0 [M+H]⁺.

Step 5. To a mixture of 2-(azetidin-3-yl)-N-(3-(difluoromethoxy)-4-fluorophenyl)acetamide (0.703 g, 2.57 mmol, 1.20 eq), 3-(4-bromo-2,6-difluorophenyl)piperidine-2,6-dione V (0.650 g, 2.14 mmol, 1.00 eq) and cesium carbonate (2.09 g, 6.41 mmol, 3.00 eq) in dioxane (10 mL) was added palladium acetate (0.0479 g, 0.214 mmol, 0.100 eq) and 4,5-bis(diphenylphosphino)-9,9-dimethylxanthene (0.247 g, 0.427 mmol, 0.200 eq) in one portion. The mixture was stirred at 100° C. for 12 h under nitrogen. The mixture was added water (80 mL), extracted with ethyl acetate (3×100 mL). The organic layer was washed with brine (30 mL), dried over anhydrous sodium sulfate, filtered and concentrated in vacuum. The residue was purified by silica gel chromatography (petroleum ether/ethyl acetate=1/1) and concentrated in vacuum to give the crude product. The crude product was purified by Prep-HPLC (column: Phenomenex Synergi C18 150*25 mm*10 μm; mobile phase: [water (formic acid)-acetonitrile]; B %: 39%-69%, 10 min) and lyophilized to give N-(3-(difluoromethoxy)-4-fluorophenyl)-2-(1-(4-(2,6-dioxopiperidin-3-yl)-3,5-difluorophenyl)azetidin-3-yl)acetamide #6 (92.74 mg, 179 umol, 8% yield, 96% purity) a white solid. ¹H NMR (400 MHz, DMSO-d₆) δ=10.87 (s, 1H), 10.23 (s, 1H), 7.78 (br d, J=6.5 Hz, 1H), 7.43-7.30 (m, 2H), 7.22 (s, 1H), 7.04 (s, 1H), 6.13 (d, J=11.1 Hz, 2H), 4.10-3.94 (m, 3H), 3.57 (br t, J=6.4 Hz, 2H), 3.11-2.96 (m, 1H), 2.86-2.76 (m, 1H), 2.73 (br d, J=7.8 Hz, 2H), 2.55 (br s, 1H), 2.14-2.01 (m, 1H), 1.99-1.90 (m, 1H). MS (ESI) m/z 498.2 [M+H]⁺.

### Example 8. Synthesis of Compound 7

To a solution of 3-(4-(3-aminoazetidin-1-yl)-2,6-difluorophenyl)piperidine-2,6-dione IX (60.0 mg, 176 μmol, 1.00 eq, formate), 2-(3-fluoro-5-methylphenyl)acetic acid (29.6 mg, 176 μmol, 1.00 eq), N,N-diisopropyl ethylamine (68.2 mg, 527 μmol, 91.9 uL, 3.00 eq) and 1-(3-dimethylaminopropyl)-3-ethylcarbodiimide hydrochlorid (43.8 mg, 229 μmol, 1.30 eq) in dimethylformamide (1 mL) was added 1-hydroxybenzotriazole (23.8 mg, 176 μmol, 1.00 eq) at 0° C. The mixture was stirred at 25° C. for 1 h. The mixture was filtered and the filtrate was purified by Prep-HPLC (column: Phenomenex Synergi C18 150*25 mm*10 μm; mobile phase: [water (formic acid)-acetonitrile]; B %: 34%-64%, 10 min) and lyophilized to afford N-(1-(4-(2,6-dioxopiperidin-3-yl)-3,5-difluorophenyl)azetidin-3-yl)-2-(3-fluoro-5-methylphenyl)acetamide #7 (34.67 mg, 77.1 μmol, 44% yield, 99% purity) as a blue solid. ¹H NMR (400 MHz, DMSO-d₆) δ=10.86 (s, 1H), 8.77 (d, J=7.0 Hz, 1H), 7.01-6.78 (m, 3H), 6.18 (d, J=11.0 Hz, 2H), 4.59-4.48 (m, 1H), 4.10 (t, J=7.8 Hz, 2H), 4.03 (dd, J=5.1, 12.8 Hz, 1H), 3.63 (dd, J=5.6, 7.8 Hz, 2H), 3.41 (s, 2H), 2.82-2.73 (m, 1H), 2.54-2.52 (m, 1H), 2.29 (s, 3H), 2.14-2.04 (m, 1H), 1.98-1.88 (m, 1H). MS (ESI) m/z 446.2 [M+H]⁺.

### Example 9 Synthesis of Compound 8

To a solution of 3-(4-(3-aminoazetidin-1-yl)-2,6-difluorophenyl)piperidine-2,6-dione IX (60.0 mg, 176 μmol, 1.00 eq, formate), 2-(3,5-difluorophenyl)acetic acid (30.3 mg, 176 μmol, 1.00 eq), N,N-diisopropylethylamine (91.9 μL, 527 μmol, 3.00 eq) and 1-(3-dimethylaminopropyl)-3-ethylcarbodiimide hydrochlorid (43.8 mg, 229 μmol, 1.30 eq) in dimethylformamide (1 mL) was added 1-hydroxybenzotriazole (23.8 mg, 176 μmol, 1.00 eq) at 0° C. The mixture was stirred at 25° C. for 1 h. The mixture was filtered. The filtrate was purified by Prep-HPLC (column: Phenomenex Synergi C18 150*25 mm*10 μm; mobile phase: [water (formic acid)-acetonitrile]; B %: 42%-60%, 9 min) and lyophilized to afford 2-(3,5-difluorophenyl)-N-(1-(4-(2,6-dioxopiperidin-3-yl)-3,5-difluorophenyl)azetidin-3-yl) acetamide #8 (43.06 mg, 94.9 μmol, 54% yield, 99% purity) as a white solid. ¹H NMR (400 MHz, DMSO-d₆) δ=10.87 (s, 1H), 8.81 (d, J=6.8 Hz, 1H), 7.10 (tt, J=2.3, 9.5 Hz, 1H), 7.05-6.91 (m, 2H), 6.18 (d, J=11.1 Hz, 2H), 4.60-4.50 (m, 1H), 4.11 (t, J=7.7 Hz, 2H), 4.04 (br dd, J=5.1, 12.4 Hz, 1H), 3.64 (dd, J=5.5, 7.7 Hz, 2H), 3.49 (s, 2H), 2.84-2.72 (m, 1H), 2.54 (br s, 1H), 2.14-2.00 (m, 1H), 1.98-1.88 (m, 1H). MS (ESI) m/z 450.1 [M+H]⁺.

### Example 10. Synthesis of Compound 9

Step 1. A mixture of 2-fluoro-5-(trifluoromethoxy)aniline (0.450 g, 2.31 mmol, 1.20 eq), 2-(1-(tert-butoxycarbonyl)azetidin-3-yl)acetic acid (0.414 g, 1.92 mmol, 1.00 eq), 0-(7-azabenzotriazol-1-yl)-N,N,N′,N′-tetramethyluronium hexafluorophosphate (0.877 g, 2.31 mmol, 1.20 eq) and N,N-diisopropylethylamine (0.745 g, 5.77 mmol, 3.00 eq) in dichloromethane (10 mL) was stirred at 25° C. for 12 h. The resulting mixture was poured into water (50 mL) and the resulting mixture was extracted with ethyl acetate (3×50 mL). The organic layer was washed with brine (30 mL), dried over sodium sulfate, filtered and concentrated under reduced pressure to give the residue which was purified by column chromatography (SiO₂, petroleum ether/ethyl acetate=2/1). The desired fraction was collected and concentrated under reduced pressure to give tert-butyl 3-(2-((2-fluoro-5-(trifluoromethoxy)phenyl)amino)-2-oxoethyl)azetidine-1-carboxylate (0.980 g, 2.22 mmol, 58% yield, 89% purity) as a light yellow solid. ¹H NMR (400 MHz, CDCl₃) δ 8.32 (d, J=3.9 Hz, 1H), 7.51 (s, 1H), 7.11 (t, J=9.7 Hz, 1H), 6.98-6.86 (m, 1H), 4.15-4.09 (m, 2H), 3.67 (dd, J=5.3, 8.7 Hz, 2H), 3.09-2.96 (m, 1H), 2.78 (d, J=7.8 Hz, 2H), 1.44 (s, 9H). MS (ESI) m/z 336.9 [M-56+H]⁺.

Step 2. A mixture of tert-butyl 3-(2-((2-fluoro-5-(trifluoromethoxy)phenyl)amino)-2-oxoethyl)azetidine-1-carboxylate (0.880 g, 2.24 mmol, 1.00 eq) and 2,2,2-trifluoroacetic acid (0.256 g, 2.24 mmol, 1.00 eq) in dichloromethane (10 mL) was stirred at 25° C. for 2 h. The reaction mixture was concentrated under reduced pressure to give 2-(azetidin-3-yl)-N-(2-fluoro-5-(trifluoromethoxy)phenyl)acetamide (1.00 g, crude) as a light yellow oil and was used to the next step without purification. MS (ESI) m/z 293.0 [M+H]⁺.

Step 3. Four reactions were carried out in parallel then pooled before purification. A mixture of 2-(azetidin-3-yl)-N-(2-fluoro-5-(trifluoromethoxy)phenyl)acetamide (0.200 g, 0.492 mmol, 1.00 eq), 3-(4-bromo-2,6-difluorophenyl)piperidine-2,6-dione V (0.150 g, 0.492 mmol, 1.00 eq), palladium(II) acetate (0.0111 g, 0.0492 mmol, 0.100 eq), 4,5-bis(diphenylphosphino)-9,9-dimethylxanthene (0.0570 g, 0.0985 mmol, 0.200 eq) and cesium carbonate (0.481 g, 1.48 mmol, 3.00 eq) in 1,4-dioxane (4 mL) was stirred at 100° C. for 12 h under argon atmosphere. The reaction mixture was cooled to room temperature. The mixture was poured into water (30 mL) and the resulting mixture was extracted with ethyl acetate (3×50 mL). The organic layer was washed with brine (30 mL), dried over sodium sulfate, filtered and concentrated reduced pressure to give a residue, which was purified by column chromatography of silica gel (petroleum ether/ethyl acetate=1/1). The desired fraction was collected and concentrated reduced pressure to give a crude product (˜0.210 g). The crude product was purified by prep-HPLC (column: Waters X bridge 150 mm*25 mm*5 μm; mobile phase: [water (ammonium bicarbonate)-acetonitrile]; B %: 47%-77%, 10 min). The desired fraction was collected and concentrated under reduced pressure to give 2-(1-(4-(2,6-dioxopiperidin-3-yl)-3,5-difluorophenyl)azetidin-3-yl)-N-(2-fluoro-5-(trifluoromethoxy)phenyl)acetamide #9 (0.105 g, 0.202 mmol, 10% yield, 99% purity) as a white solid. ¹H NMR (400 MHz, DMSO-d₆) δ 10.85 (s, 1H), 10.06 (s, 1H), 8.11 (dd, J=2.8, 5.9 Hz, 1H), 7.41 (dd, J=9.0, 10.4 Hz, 1H), 7.18-7.11 (m, 1H), 6.14 (s, 1H), 6.11 (s, 1H), 4.01 (q, J=7.8 Hz, 3H), 3.56 (t, J=6.1 Hz, 2H), 3.30 (s, 1H), 3.11-2.97 (m, 1H), 2.83 (d, J=7.8 Hz, 2H), 2.80-2.71 (m, 1H), 2.13-1.99 (m, 1H), 1.98-1.87 (m, 1H). ¹H NMR (400 MHz, CDCl3) δ 8.34 (d, J=3.9 Hz, 1H), 7.93 (s, 1H), 7.46 (s, 1H), 7.16-7.08 (m, 1H), 6.95 (dd, J=3.9, 8.2 Hz, 1H), 5.97 (s, 1H), 5.95 (s, 1H), 4.12 (dt, J=2.2, 7.7 Hz, 2H), 3.95 (dd, J=5.0, 12.3 Hz, 1H), 3.62 (t, J=5.9 Hz, 2H), 3.29-3.15 (m, 1H), 2.84 (d, J=7.8 Hz, 2H), 2.79 (t, J=3.7 Hz, 1H), 2.73-2.62 (m, 1H), 2.32 (dq, J=4.3, 12.9 Hz, 1H), 2.18-2.09 (m, 1H). MS (ESI) m/z 516.1 [M+H]⁺.

### Example 11. Synthesis of Compound 10

Step 1. To a mixture of 2-bromo-5-chloro-pyridine (2.00 g, 10.3 mmol, 1.00 eq), cesium carbonate (6.77 g, 20.7 mmol, 2.00 eq), copper iodide (197 mg, 1.04 mmol, 0.100 eq) and picolinic acid (255 mg, 2.08 mmol, 0.200 eq) in 1,4-dioxane (20 mL) was added diethyl propanedioate (3.33 g, 20.7 mmol, 2.00 eq). After stirring at 110° C. for 12 h, the reaction mixture was poured into saturated ammonium chloride aqueous solution (100 mL) and extracted with ethyl acetate (3×30 mL). The organic phase was concentrated under reduced pressure to give a residue which was purified by column chromatography (SiO₂, petroleum ether/ethyl acetate=20/1 to 10/1) to afford diethyl 2-(5-chloro-2-pyridyl)propanedioate (420 mg, crude) as colorless oil. MS (ESI) m/z 272.0 [M+H]⁺.

Step 2. To a mixture of diethyl 2-(5-chloro-2-pyridyl)propanedioate (420 mg, 1.55 mmol, 1.00 eq) in methanol (10 mL) and water (5 mL) was added lithium hydroxide (185 mg, 7.73 mmol, 5.00 eq). After stirring at 25° C. for 12 h, the reaction mixture was poured into water (20 mL) and adjusted pH to 5-6 with hydrochloric acid (1 M). The mixture was extracted with ethyl acetate (5×10 mL), washed with brine, dried over anhydrous sodium sulfate and concentrated under reduced pressure to afford 2-(5-chloro-2-pyridyl)acetic acid (210 mg, 71% yield, 90% purity) as a white solid. ¹H NMR (400 MHz, DMSO-d₆) δ=12.47 (br s, 1H), 8.53 (d, J=2.4 Hz, 1H), 7.88 (dd, J=2.4, 8.4 Hz, 1H), 7.40 (d, J=8.4 Hz, 1H), 3.76 (s, 2H).

Step 3. To a solution of 2-(5-chloro-2-pyridyl)acetic acid (60.0 mg, 0.315 mmol, 90% purity, 1.00 eq) in N,N-dimethylformamide (1 mL) were added N-ethyl-N-propan-2-ylpropan-2-amine (122 mg, 0.944 mmol, 0.160 mL, 3.00 eq), 2,4,6-tripropyl-1,3,5,2,4,6-trioxatriphosphinane 2,4,6-trioxide (0.380 mL, 0.630 mmol, 50% purity, 2.00 eq) and 3-(4-(3-aminoazetidin-1-yl)-2,6-difluoro-phenyl)piperidine-2,6-dione (103 mg, 0.315 mmol, 90% purity, 1.00 eq) at 25° C. The mixture was stirred at 25° C. for 2 h. The reaction mixture was poured into water (8 mL) and then extracted with ethyl acetate (3×8 mL), the combined organic phase was washed with brine (3×5 mL), dried with anhydrous sodium sulfate, filtered and the filtrate was concentrated under reduced pressure to give a residue. The residue was purified by prep-HPLC (FA condition; column: Phenomenex Luna C18 150*25 mm*10 μm; mobile phase: [water (formic acid)-acetonitrile]; B %: 26%-56%, 10 min) to afford 2-(5-chloropyridin-2-yl)-N-(1-(4-(2,6-dioxopiperidin-3-yl)-3,5-difluorophenyl)azetidin-3-yl)acetamide #10 (10.0 mg, 0.0218 mmol, 7% yield, 98% purity, formate) as a yellow solid. ¹H NMR (400 MHz, DMSO-d₆) δ=10.86 (s, 1H), 8.85 (d, J=7.2 Hz, 1H), 8.52 (d, J=2.8 Hz, 1H), 7.87 (dd, J=8.4, 2.4 Hz, 1H), 7.39 (d, J=8.4 Hz, 1H), 6.17 (d, J=11.2 Hz, 2H), 4.51-4.61 (m, 1H), 4.11 (t, J=7.6 Hz, 2H), 4.03 (dd, J=12.8, 4.8 Hz, 1H), 3.60-3.69 (m, 4H), 2.71-2.84 (m, 1H), 1.96-2.14 (m, 2H), 1.87-1.96 (m, 1H). MS (ESI) m/z. 449.1 [M+H]⁺.

### Example 12. Synthesis of Compound 11

To a solution of 3-(4-(3-aminoazetidin-1-yl)-2,6-difluorophenyl)piperidine-2,6-dione IX (80.0 mg, 234 μmol, 1.00 eq), 2-(2,4-difluorophenyl)acetic acid (40.4 mg, 234 μmol, 1.00 eq), N,N-diisopropylethylamine (122 μL, 703 μmol, 3.00 eq) and 1-(3-dimethylaminopropyl)-3-ethylcarbodiimide hydrochlorid (58.4 mg, 305 μmol, 1.30 eq) in dimethylformamide (1 mL) was added 1-hydroxybenzotriazole (31.7 mg, 234 μmol, 1.00 eq) at 0° C. The mixture was stirred at 25° C. for 2 h. The mixture was filtered. The filtrate was purified by Prep-HPLC (column: Phenomenex luna C18 150*25 mm*10 μm; mobile phase: [water (formic acid)-acetonitrile]; B %: 29%-59%, 10 min) and lyophilized to afford 2-(2,4-difluorophenyl)-N-(1-(4-(2,6-dioxopiperidin-3-yl)-3,5-difluorophenyl)azetidin-3-yl)acetamide #11 (38.86 mg, 85.6 umol, 37% yield, 99% purity) as an off-white solid. ¹H NMR (400 MHz, DMSO-d₆) δ=10.86 (s, 1H), 8.78 (d, J=7.0 Hz, 1H), 7.42-7.32 (m, 1H), 7.18 (dt, J=2.6, 9.8 Hz, 1H), 7.03 (dt, J=2.0, 8.6 Hz, 1H), 6.18 (d, J=11.0 Hz, 2H), 4.60-4.52 (m, 1H), 4.11 (t, J=7.7 Hz, 2H), 4.04 (dd, J=5.1, 12.7 Hz, 1H), 3.64 (dd, J=5.5, 7.8 Hz, 2H), 3.49 (s, 2H), 2.84-2.72 (m, 1H), 2.52 (m, 1H), 2.07 (m, 1H), 1.98-1.89 (m, 1H). MS (ESI) m/z 450.1 [M+H]⁺.

### Example 13. Synthesis of Compound 12

To a solution of 3-(4-(3-aminoazetidin-1-yl)-2,6-difluorophenyl)piperidine-2,6-dione IX (80.0 mg, 271 μmol, 1.00 eq), 2-(5-chloro-2-fluorophenyl)acetic acid (51.1 mg, 271 μmol, 1.00 eq), 1-(3-dimethylaminopropyl)-3-ethylcarbodiimide hydrochlorid (67.5 mg, 352 μmol, 1.30 eq) and N,N-diisopropylethylamine (142 μL, 813 μmol, 3.00 eq) in dimethylformamide (1 mL) was added 1-hydroxybenzotriazole (36.6 mg, 271 μmol, 1.00 eq) at 0° C. The mixture was stirred at 25° C. for 2 h. The mixture was filtered. The filtrate was purified by Prep-HPLC (column: Phenomenex Synergi C18 150*25 mm*10 μm; mobile phase: [water (formic acid)-acetonitrile]; B %: 35%-65%, 10 min) and lyophilized to afford 2-(5-chloro-2-fluorophenyl)-N-(1-(4-(2,6-dioxopiperidin-3-yl)-3,5-difluorophenyl) azetidin-3-yl)acetamide #12 (72.88 mg, 153 μmol, 57% yield, 98% purity) as a white solid. ¹H NMR (400 MHz, DMSO-d₆) δ=10.86 (s, 1H), 8.81 (d, J=7.1 Hz, 1H), 7.43 (dd, J=2.7, 6.5 Hz, 1H), 7.39-7.32 (m, 1H), 7.25-7.18 (m, 1H), 6.18 (d, J=11.0 Hz, 2H), 4.60-4.51 (m, 1H), 4.11 (t, J=7.7 Hz, 2H), 4.04 (dd, J=5.1, 12.7 Hz, 1H), 3.64 (dd, J=5.7, 7.8 Hz, 2H), 3.52 (s, 2H), 2.82-2.73 (m, 1H), 2.53-2.52 (m, 1H), 2.14-2.03 (m, 1H), 1.99-1.90 (m, 1H). MS (ESI) m/z 466.1 [M+H]⁺.

### Example 14. Synthesis of Compound 13

To a solution of 3-(4-(3-aminoazetidin-1-yl)-2,6-difluorophenyl)piperidine-2,6-dione IX (80.0 mg, 271 μmol, 1.00 eq), 2-(3-chloro-4-fluorophenyl)acetic acid (51.1 mg, 271 μmol, 1.00 eq), 1-(3-dimethylaminopropyl)-3-ethylcarbodiimide hydrochlorid (67.5 mg, 352 umol, 1.30 eq) and N,N-diisopropylethylamine (142 μL, 813 umol, 3.00 eq) in dimethylformamide (1 mL) was added 1-hydroxybenzotriazole (36.6 mg, 271 μmol, 1.00 eq) at 0° C. The mixture was stirred at 25° C. for 2 h. The mixture was filtered. The filtrate was purified by prep-HPLC (column: Phenomenex Synergi C18 150*25 mm*10 μm; mobile phase: [water (formic acid)-acetonitrile]; B %: 35%-65%, 10 min) and lyophilized to afford 2-(3-chloro-4-fluorophenyl)-N-(1-(4-(2,6-dioxopiperidin-3-yl)-3,5-difluorophenyl) azetidin-3-yl)acetamide #13 (67.11 mg, 140 μmol, 52% yield, 97% purity) as a white solid. ¹H NMR (400 MHz, DMSO-d₆) δ=10.86 (s, 1H), 8.77 (d, J=6.8 Hz, 1H), 7.47 (dd, J=2.1, 7.2 Hz, 1H), 7.39-7.30 (m, 1H), 7.29-7.21 (m, 1H), 6.18 (d, J=11.0 Hz, 2H), 4.59-4.49 (m, 1H), 4.10 (t, J=7.7 Hz, 2H), 4.04 (dd, J=5.0, 12.5 Hz, 1H), 3.63 (dd, J=5.5, 7.8 Hz, 2H), 3.45 (s, 2H), 2.82-2.73 (m, 1H), 2.54-2.53 (m, 1H), 2.14-2.02 (m, 1H), 1.98-1.91 (m, 1H). MS (ESI) m/z 466.1 [M+H]⁺.

### Example 15. Synthesis of Compound 14

To a solution of 3-(4-(3-aminoazetidin-1-yl)-2,6-difluorophenyl)piperidine-2,6-dione IX (80.0 mg, 271 μmol, 1.00 eq), 2-(4-chloro-3-fluoro-phenyl)acetic acid (51.1 mg, 271 μmol, 1.00 eq), 1-(3-dimethylaminopropyl)-3-ethylcarbodiimide hydrochlorid (67.5 mg, 352 μmol, 1.30 eq) and N,N-diisopropylethylamine (142 μL, 813 μmol, 3.00 eq) in dimethylformamide (1 mL) was added 1-hydroxybenzotriazole (36.6 mg, 271 μmol, 1.00 eq) at 0° C. The mixture was stirred at 25° C. for 2 h. The mixture was filtered. The filtrate was purified by prep-HPLC (column: Phenomenex Synergi C18 150*25 mm*10 μm; mobile phase: [water (formic acid)-acetonitrile]; B %: 35%-65%, 10 min) and lyophilized to afford 2-(4-chloro-3-fluorophenyl)-N-(1-(4-(2,6-dioxopiperidin-3-yl)-3,5-difluorophenyl) azetidin-3-yl)acetamide #14 (66.25 mg, 138 μmol, 51% yield, 97% purity) as a white solid. ¹H NMR (400 MHz, DMSO-d₆) δ=10.86 (s, 1H), 8.78 (d, J=7.0 Hz, 1H), 7.51 (t, J=8.1 Hz, 1H), 7.30 (dd, J=1.9, 10.5 Hz, 1H), 7.12 (dd, J=1.6, 8.2 Hz, 1H), 6.17 (d, J=11.0 Hz, 2H), 4.59-4.50 (m, 1H), 4.10 (t, J=7.7 Hz, 2H), 4.03 (br dd, J=4.9, 12.5 Hz, 1H), 3.63 (dd, J=5.4, 7.9 Hz, 2H), 3.48 (s, 2H), 2.83-2.73 (m, 1H), 2.52 (br s, 1H), 2.13-2.03 (m, 1H), 1.98-1.90 (m, 1H). MS (ESI) m/z 466.1 [M+H]⁺.

### Example 16. Synthesis of Compound 15

Step 1. A mixture of 2-(5-chloro-3-fluoropyridin-2-yl)acetonitrile (200 mg, 1.17 mmol, 1.00 eq) in sulfuric acid (1 mL) and water (1 mL) was stirred at 100° C. for 2 h. The mixture was quenched by saturated sodium carbonate (50 mL), extracted with ethyl acetate (4×50 mL). The organic layers were washed with brine (40 mL), dried over anhydrous sodium sulfate, filtered and concentrated to afford 2-(5-chloro-3-fluoropyridin-2-yl)acetic acid (100 mg, 0.528 mmol, 45% yield) as a brown solid. ¹H NMR (400 MHz, DMSO-d₆) δ=12.95-12.53 (m, 1H), 8.46 (d, J=1.1 Hz, 1H), 8.09 (dd, J=1.9, 9.5 Hz, 1H), 3.82 (d, J=2.4 Hz, 2H).

Step 2. To a solution of 3-(4-(3-aminoazetidin-1-yl)-2,6-difluorophenyl)piperidine-2,6-dione IX (90.0 mg, 264 μmol, 1.00 eq, formate), 2-(5-chloro-3-fluoropyridin-2-yl)acetic acid (50.0 mg, 264 μmol, 1.00 eq), N,N-diisopropylethylamine (139 μL, 791 μmol, 3.00 eq) and 1-(3-dimethylaminopropyl)-3-ethylcarbodiimide hydrochlorid (65.7 mg, 343 μmol, 1.30 eq) in dimethylformamide (1 mL) was added 1-hydroxybenzotriazole (35.6 mg, 264 μmol, 1.00 eq) at 0° C. The mixture was stirred at 20° C. for 12 h. The mixture was filtered. The filtrate was purified by prep-HPLC (column: Phenomenex Synergi C18 150*25 mm*10 μm; mobile phase: [water (formic acid)-acetonitrile]; B %: 33%-53%, 10 min) and lyophilized to afford 2-(5-chloro-3-fluoropyridin-2-yl)-N-(1-(4-(2,6-dioxopiperidin-3-yl)-3,5-difluorophenyl) azetidin-3-yl)acetamide #15 (26.56 mg, 54.6 μmol, 21% yield, 96% purity) as a white solid. ¹H NMR (400 MHz, DMSO-d₆) δ=10.88 (s, 1H), 8.88 (d, J=7.0 Hz, 1H), 8.45 (d, J=1.3 Hz, 1H), 8.06 (dd, J=2.1, 9.4 Hz, 1H), 6.19 (d, J=11.1 Hz, 2H), 4.60-4.52 (m, 1H), 4.11 (t, J=7.7 Hz, 2H), 4.04 (br dd, J=5.1, 12.8 Hz, 1H), 3.72 (d, J=2.2 Hz, 2H), 3.65 (dd, J=5.6, 7.8 Hz, 2H), 2.82-2.73 (m, 1H), 2.52 (br s, 1H), 2.13-2.03 (m, 1H), 1.98-1.91 (m, 1H). MS (ESI) m/z 467.1 [M+H]⁺.

### Example 17. Synthesis of Compound 16

Step 1. A mixture of 2-(4-chloro-2,6-difluorophenyl)acetonitrile (300 mg, 1.60 mmol, 1.00 eq) in sulfuric acid (10 mL) and water (10 mL) was stirred at 100° C. for 12 h. The reaction mixture was extracted with ethyl acetate (5×30 mL). The organic layers were washed with brine (30 mL), dried over anhydrous sodium sulfate, filtered and concentrated to afford 2-(4-chloro-2,6-difluorophenyl)acetic acid (200 mg, 0.968 mmol, 61% yield) as a white solid and was used to the next step without purification. ¹H NMR (400 MHz, DMSO-d₆) δ=13.08-12.38 (m, 1H), 7.51-7.17 (m, 2H), 3.62 (s, 2H).

Step 2. To a solution of 3-(4-(3-aminoazetidin-1-yl)-2,6-difluorophenyl)piperidine-2,6-dione IX (80.0 mg, 234 μmol, 1.00 eq, formate), 2-(4-chloro-2,6-difluorophenyl)acetic acid (48.4 mg, 234 μmol, 1.00 eq), 1-(3-dimethylaminopropyl)-3-ethylcarbodiimide hydrochlorid (58.4 mg, 305 μmol, 1.30 eq) and N,N-diisopropylethylamine (122 μL, 703 μmol, 3.00 eq) in dimethylformamide (1 mL) was added 1-hydroxybenzotriazole (31.7 mg, 234 μmol, 1.00 eq) at 0° C. The mixture was stirred at 25° C. for 2 h. The mixture was filtered. The filtrate was purified by prep-HPLC (column: Phenomenex Synergi C18 150*25 mm*10 μm; mobile phase: [water (formic acid)-acetonitrile]; B %: 36%-66%, 10 min) and lyophilized to afford 2-(4-chloro-2,6-difluorophenyl)-N-(1-(4-(2,6-dioxopiperidin-3-yl)-3,5-difluorophenyl)azetidin-3-yl) acetamide #16 (46.6 mg, 95.4 μmol, 41% yield, 99% purity) as a white solid. ¹H NMR (400 MHz, DMSO-d₆) δ=10.86 (s, 1H), 8.86 (d, J=6.9 Hz, 1H), 7.49-7.21 (m, 2H), 6.19 (s, 1H), 6.17 (s, 1H), 4.60-4.51 (m, 1H), 4.11 (t, J=7.7 Hz, 2H), 4.04 (dd, J=5.0, 12.5 Hz, 1H), 3.65 (dd, J=5.5, 8.0 Hz, 2H), 3.52 (s, 2H), 2.83-2.73 (m, 1H), 2.53-2.52 (m, 1H), 2.13-2.03 (m, 1H), 1.98-1.90 (m, 1H). MS (ESI) m/z 484.1 [M+H]⁺.

### Example 18. Synthesis of Compound 17

To a solution of 3-(4-(3-aminoazetidin-1-yl)-2,6-difluorophenyl)piperidine-2,6-dione IX (80.0 mg, 234 μmol, 1.00 eq, formate), 2-(3,4-difluorophenyl)acetic acid (40.4 mg, 234 μmol, 1.00 eq), 1-(3-dimethylaminopropyl)-3-ethylcarbodiimide hydrochlorid (58.4 mg, 305 μmol, 1.30 eq) and N,N-diisopropylethylamine (122 μL, 703 μmol, 3.00 eq) in dimethylformamide (1 mL) was added 1-hydroxybenzotriazole (31.7 mg, 234 μmol, 1.00 eq) at 0° C. The mixture was stirred at 25° C. for 2 h. The mixture was filtered. The filtrate was purified by prep-HPLC (column: Phenomenex Synergi C18 150*25 mm*10 μm; mobile phase: [water (formic acid)-acetonitrile]; B %: 32%-62%, 10 min) and lyophilized to afford 2-(3,4-difluorophenyl)-N-(1-(4-(2,6-dioxopiperidin-3-yl)-3,5-difluorophenyl)azetidin-3-yl)acetamide #17 (39.39 mg, 86.8 μmol, 37% yield, 99% purity) as a white solid. ¹H NMR (400 MHz, DMSO-d₆) δ=10.86 (s, 1H), 8.77 (d, J=7.0 Hz, 1H), 7.39-7.28 (m, 2H), 7.09 (ddd, J=2.1, 4.1, 6.1 Hz, 1H), 6.19 (s, 1H), 6.16 (s, 1H), 4.57-4.50 (m, 1H), 4.10 (t, J=7.7 Hz, 2H), 4.03 (dd, J=5.1, 12.8 Hz, 1H), 3.63 (dd, J=5.5, 8.0 Hz, 2H), 3.45 (s, 2H), 2.82-2.73 (m, 1H), 2.52 (br d, J=1.9 Hz, 1H), 2.12-2.03 (m, 1H), 1.98-1.92 (m, 1H). MS (ESI) m/z 450.1 [M+H]⁺.

### Example 19. Synthesis of Compound 18

Step 1. To a mixture of ethyl 2-(p-tolyl)acetate (9.00 g, 50.5 mmol, 1.00 eq) in trifluoroacetic acid (100 mL) was added hexamethylenetetraamine (7.10 g, 50.7 mmol, 1.00 eq) at 0° C. during a period of 10 min. After stirring at 80° C. for 16 h, the mixture was poured into water (300 mL) and extracted with ethyl acetate (3×300 mL). The combined organic layers were washed with brine, dried over anhydrous sodium sulfate, and filtered. The filtrate was concentrated under vacuum to give a residue. The residue was purified by column chromatography (SiO₂, petroleum ether/ethyl acetate=50/1 to 10/1) to afford ethyl 2-(3-formyl-4-methylphenyl)acetate (0.77 g, 3.73 mmol, 7% yield) as a light yellow oil. ¹H NMR (400 MHz, CDCl₃) δ=10.19 (s, 1H), 7.64 (s, 1H), 7.34 (dd, J=1.2, 8.0 Hz, 1H), 7.18 (d, J=8.0 Hz, 1H), 4.09 (q, J=7.2 Hz, 2H), 3.58 (s, 2H), 2.58 (s, 3H), 1.21-1.17 (t, J=7.2 Hz, 3H).

Step 2. A mixture of ethyl 2-(3-formyl-4-methylphenyl)acetate (670 mg, 2.60 mmol, 80% purity, 1.00 eq), acetic acid (223 μL, 3.90 mmol, 1.50 eq) and morpholine (274 μL, 3.12 mmol, 1.20 eq) in methanol (10 mL) was stirred at 20° C. for 30 min. Then sodium cyanotrihydroborate (245 mg, 3.90 mmol, 1.50 eq) was added to the mixture in portions at 0° C. After stirring at 20° C. for 16 h, the mixture was poured into water (50 mL) and extracted with ethyl acetate (3×50 mL). The combined organic layers were washed with brine, dried over anhydrous sodium sulfate and filtered. The filtrate was concentrated under vacuum to give a residue. The residue was purified by column chromatography (SiO₂, petroleum ether/ethyl acetate=20/1 to 5/1) to afford ethyl 2-(4-methyl-3-(morpholinomethyl)phenyl)acetate (500 mg, 1.53 mmol, 59% yield, 85% purity) as a colorless oil. ¹H NMR (400 MHz, DMSO-d₆) δ=7.14-7.09 (m, 2H), 7.06-7.03 (m, 1H), 4.09-4.04 (m, 2H), 3.59 (s, 2H), 3.55 (t, J=4.8 Hz, 4H), 3.40 (s, 2H), 2.38-2.32 (m, 4H), 2.30 (s, 3H), 1.19-1.15 (m, 3H).

Step 3. To a solution of ethyl ethyl 2-(4-methyl-3-(morpholinomethyl)phenyl)acetate (500 mg, 1.53 mmol, 85% purity, 1.00 eq) in sulfuric acid (10 mL) was added 1-chloropyrrolidine-2,5-dione (266 mg, 1.99 mmol, 1.30 eq). The mixture was stirred at 50° C. for 40 h. The mixture was poured into saturated sodium bicarbonate solution (300 mL) and extracted with ethyl acetate (3×50 mL). The aqueous phase was concentrated under vacuum to give a residue. The residue was washed with methanol (30 mL) and filtered. The filtrate was concentrated under vacuum to give a residue. The residue was purified by Prep-HPLC (column: Phenomenex Synergi C18; mobile phase: [water (0.225% formic acid)-acetonitrile]; B %: 0%-30%, 9 min) and concentrated under vacuum to give 2-(3-chloro-4-methyl-5-(morpholinomethyl)phenyl)acetic acid (150 mg, 476 μmol, 31% yield, 90% purity) as a colorless gum. ¹H NMR (400 MHz, DMSO-d₆) δ=12.48 (s, 1H), 7.28 (s, 1H), 7.14 (s, 1H), 3.61-3.42 (m, 8H), 2.44-2.23 (m, 4H), 2.35 (s, 3H). MS (ESI) m/z 284.0 [M+H]⁺.

Step 4. A mixture of 2-(3-chloro-4-methyl-5-(morpholinomethyl)phenyl)acetic acid (100 mg, 352 μmol, 1.00 eq), 3-(4-(3-aminoazetidin-1-yl)-2,6-difluorophenyl)piperidine-2,6-dione IX (104 mg, 352 umol, 1.00 eq), 1-propanephosphonic anhydride (314 uL, 529 umol, 50% purity in ethyl acetate, 1.50 eq) and N,N-diisopropylethylamine (123 μL, 705 μmol, 2.00 eq) in N,N-dimethylformamide (1 mL) was stirred at 20° C. for 12 h. The mixture was poured into water (10 mL) and extracted with ethyl acetate (3×20 mL). The combined organic layers were washed with brine, dried over anhydrous sodium sulfate, filtered and concentrated under vacuum to give a residue. The residue was purified by Prep-HPLC (column: Phenomenex C18 75*30 mm*3 μm; mobile phase: [water (formic acid)-acetonitrile]; B %: 10%-40%, 7 min), Prep-HPLC (column: Waters Xbridge 150*25 mm*5 μm; mobile phase: [water (ammonium bicarbonate)-acetonitrile]; B %: 38%-68%, 10 min) and lyophilized to afford 2-(3-chloro-4-methyl-5-(morpholino methyl)phenyl)-N-(1-(4-(2,6-dioxopiperidin-3-yl)-3,5-difluorophenyl)azetidin-3-yl)acetamide #18 (45.22 mg, 79.7 μmol, 23% yield, 99% purity) as white solid. ¹H NMR (400 MHz, DMSO-d₆) δ=10.85 (s, 1H), 8.73 (d, J=6.8 Hz, 1H), 7.24 (d, J=1.2 Hz, 1H), 7.10 (d, J=1.2 Hz, 1H), 6.17 (d, J=11.2 Hz, 2H), 4.58-4.48 (m, 1H), 4.10 (t, J=8.4 Hz, 2H), 4.06-3.99 (m, 1H), 3.63-3.58 (m, 2H), 3.54 (t, J=4.4 Hz, 4H), 3.42 (s, 2H), 3.39 (s, 2H), 2.83-2.73 (m, 1H), 2.59-2.56 (m, 1H), 2.36-2.32 (m, 7H), 2.13-2.01 (m, 1H), 1.98-1.90 (m, 1H). MS (ESI) m/z 561.2 [M+H]⁺.

### Example 20. Synthesis of Compound 19

Step 1. To a mixture of 2-(6-chloro-3-pyridyl)acetic acid (2.00 g, 11.7 mmol, 1.00 eq) in methanol (20 mL) was added thionyl dichloride (1.69 mL, 2.77 g, 23.3 mmol 2.00 eq) dropwise at 20° C. After addition, the mixture was stirred at 40° C. for 16 h. The mixture was concentrated under vacuum to give a residue. The residue was poured into saturated sodium bicarbonate aqueous solution (100 mL) and extracted with ethyl acetate (3×50 mL). The organic layer was washed with brine (50 mL), dried over anhydrous sodium sulfate and filtered. The filtrate was concentrated under vacuum to afford methyl 2-(6-chloro-3-pyridyl)acetate (1.90 g, 9.72 mmol, 83% yield, 95% purity) as a yellow oil and was used to next step directly without purification. ¹H NMR (400 MHz, DMSO-d₆) δ=8.32 (d, J=2.4 Hz, 1H), 7.79 (dd, J=2.4, 8.0 Hz, 1H), 7.50 (d, J=8.0 Hz, 1H), 3.79 (s, 2H), 3.64 (s, 3H).

Step 2. A mixture of methyl 2-(6-chloropyridin-3-yl)acetate (0.80 g, 4.09 mmol, 95% purity, 1.00 eq), (R)-2-methylpyrrolidine (418 mg, 4.91 mmol, 1.20 eq), cesium carbonate (4.00 g, 12.3 mmol, 3.00 eq) and chloro(2-dicyclohexylphosphino-2′,4′,6′-triisopropyl-1,1′-biphenyl)[2-(2′-amino-1,1′-biphenyl)]palladium (II) (322 mg, 0.409 mmol, 0.10 eq) in N,N-dimethylformamide (10 mL) was degassed under vacuum and purged 3 times with nitrogen. After stirring the mixture at 110° C. for 16 h under nitrogen atmosphere, the mixture was poured into water (20 mL), adjusted pH=7-8 with hydrochloric acid (1 M) and extracted with ethyl acetate (3×20 mL). The separated organic layer was washed with brine, dried over sodium sulfate, filtered and concentrated to give a residue. The crude product was purified by reverse phase column chromatography ([0.1% ammonium hydroxyde-acetonitrile]; B %: 0%-25%, 10 min) and lyophilized to afford (R)-2-(6-(2-methylpyrrolidin-1-yl)pyridin-3-yl)acetic acid (200 mg, 0.817 mmol, 20% yield, 90% purity) as a colorless gum. ¹H NMR (400 MHz, DMSO-d₆) δ=7.87 (d, J=2.4 Hz, 1H), 7.34 (dd, J=2.4, 8.4 Hz, 1H), 6.36 (d, J=8.4 Hz, 1H), 4.12-4.02 (m, 1H), 3.48-3.40 (m, 1H), 3.27 (s, 2H), 3.24-3.17 (m, 1H), 2.04-1.95 (m, 2H), 1.93-1.87 (m, 1H), 1.67-1.61 (m, 1H), 1.13 (d, J=6.0 Hz, 3H). MS (ESI) m/z 221.2 [M+H]⁺.

Step 3. A mixture of 3-(4-(3-aminoazetidin-1-yl)-2,6-difluorophenyl)piperidine-2,6-dione IX (100 mg, 339 μmol, 1.00 eq), (R)-2-(6-(2-methylpyrrolidin-1-yl)pyridin-3-yl)acetic acid (82.9 mg, 339 μmol, 90% purity, 1.00 eq), 1-propanephosphonic anhydride (302 μL, 508 μmol, 50% purity in ethyl acetate, 1.50 eq) and N,N-diisopropylethylamine (118 μL, 677 umol, 2.00 eq) in N,N-dimethylformamide (2 mL) was stirred at 25° C. for 12 h. The mixture was poured into water (10 mL) and extracted with ethyl acetate (3×20 mL). The organic layer was washed with brine, dried over anhydrous sodium sulfate, filtered and concentrated under vacuum to give a residue. The residue was purified by Prep-HPLC (column: Waters Xbridge 150*25 mm*5 μm; mobile phase: [water(ammonium bicarbonate)-acetonitrile]; B %: 33%-63%, 10 min) and Prep-HPLC (column: Waters Xbridge 150*25 mm*5 μm; mobile phase: [water(ammonium bicarbonate)-acetonitrile]; B %: 33%-63%, 10 min) and lyophilized to afford N-(1-(4-(2,6-dioxopiperidin-3-yl)-3,5-difluorophenyl)azetidin-3-yl)-2-(6-((R)-2-methylpyrrolidin-1-yl)pyridin-3-yl)acetamide #19 (47.24 mg, 94.0 μmol, 27.8% yield, 99% purity) as a white solid. ¹H NMR (400 MHz, DMSO-d₆) δ=10.85 (s, 1H), 8.65 (d, J=6.8 Hz, 1H), 7.90 (d, J=2.4 Hz, 1H), 7.35 (dd, J=2.4, 8.8 Hz, 1H), 6.37 (d, J=8.8 Hz, 1H), 6.16 (d, J=11.2 Hz, 2H), 4.58-4.48 (m, 1H), 4.12-4.00 (m, 4H), 3.65-3.58 (m, 2H), 3.46-3.39 (m, 1H), 3.24 (s, 2H), 3.22-3.15 (m, 1H), 2.82-2.72 (m, 1H), 2.48-2.45 (m, 1H), 2.13-1.87 (m, 5H), 1.69-1.60 (m, 1H), 1.12 (d, J=6.0 Hz, 3H). MS (ESI) m/z 498.2 [M+H]⁺.

### Example 21. Synthesis of Compound 20

Step 1. To a solution of 1,4-dibromobenzene (5.43 mL, 42.4 mmol, 1.00 eq) in tetrahydrofuran (200 mL) was added n-butyllithium (2.5 mol/L in n-hexane, 18.7 mL, 1.10 eq) at −78° C. under nitrogen atmosphere. The reaction was stirred at −78° C. for 30 min, and then cyclobutanone (3.33 mL, 44.5 mmol, 1.05 eq) was added to the mixture at −78° C. After stirring at 25° C. for 2 h, the reaction mixture was poured into saturated ammonium chloride aqueous solution (200 mL) at 0° C. and extracted with ethyl acetate (3×50 mL). The organic layer was washed with brine (50 mL), dried over anhydrous sodium sulfate, filtered and concentrated under reduced pressure to give a residue. The residue was purified by column chromatography (SiO₂, petroleum ether/ethyl acetate=10/1 to 3/1) to afford 1-(4-bromophenyl)cyclobutan-1-ol (5.2 g, crude) as colorless oil. ¹H NMR (400 MHz, DMSO-d₆) δ=7.51 (d, J=8.8 Hz, 2H), 7.43 (d, J=8.8 Hz, 2H), 5.58 (s, 1H), 2.42-2.19 (m, 4H), 2.00-1.82 (m, 1H), 1.70-1.56 (m, 1H).

Step 2. A mixture of 1-(4-bromophenyl)cyclobutan-1-ol (3.40 g, 15.0 mmol, 1.00 eq), triethylsilane (12.0 mL, 74.9 mmol, 5.00 eq) and 2,2,2-trifluoroacetic acid (11.1 mL, 17.1 g, 150 mmol, 10.0 eq) in dichloromethane (35 mL) was degassed under vacuum and purged 3 times with nitrogen. After stirring at 25° C. for 12 h under nitrogen atmosphere, the reaction mixture was quenched by addition saturated sodium bicarbonate aqueous solution (100 mL) at 0° C. and extracted with ethyl acetate (2×80 mL). The combined organic layers were washed with brine (50 mL), dried over anhydrous sodium sulfate, filtered and concentrated under vacuum to give a residue. The residue was purified by column chromatography (SiO₂, petroleum ether then petroleum ether/ethyl acetate=3/1) to afford 1-bromo-4-cyclobutylbenzene (3.9 g, crude) as colorless oil. ¹H NMR (400 MHz, DMSO-d₆) δ=7.46 (d, J=8.8 Hz, 2H), 7.18 (d, J=8.8 Hz, 2H), 3.55-3.41 (m, 1H), 2.34-2.19 (m, 2H), 2.14-1.88 (m, 3H), 1.84-1.73 (m, 1H).

Step 3. A mixture of 1-bromo-4-cyclobutyl-benzene (700 mg, 3.32 mmol, 1.00 eq), potassium 3-methoxy-3-oxopropanoate (777 mg, 4.97 mmol, 1.50 eq), 2,2′-bis(diphenylphosphaneyl)-1,1′-binaphthalene (124 mg, 0.199 mmol, 0.060 eq), diallyldipalladium dichloride (12.1 mg, 0.0663 mmol, 0.020 eq) and N,N-dimethylpyridin-4-amine (40.5 mg, 332 μmol, 0.100 eq) in mesitylene (20 mL) was degassed under vacuum and purged 3 times with nitrogen. After stirring at 130° C. for 12 h, the mixture was filtered. The filtrate was concentrated under vacuum to give a residue. The residue was purified by column chromatography (SiO₂, petroleum ether then petroleum ether/ethyl acetate=2/1) to afford methyl 2-(4-cyclobutylphenyl)acetate (170 mg, crude) as a light yellow solid. ¹H NMR (400 MHz, DMSO-d₆) δ=7.23-7.11 (m, 4H), 3.62 (s, 2H), 3.60 (s, 3H), 3.54-3.42 (m, 1H), 2.32-2.20 (m, 2H), 2.11-2.00 (m, 2H), 2.00-1.90 (m, 1H), 1.86-1.73 (m, 1H).

Step 4. To a solution of methyl 2-(4-cyclobutylphenyl)acetate (170 mg, 832 μmol, 1.00 eq) in methanol (5 mL) and water (1 mL) was added lithium hydroxide monohydrate (175 mg, 4.16 mmol, 5.00 eq). The mixture was stirred at 25° C. for 12 h. The pH of reaction mixture was adjusted to 4-5 with hydrochloric acid aqueous solution (1 M) and extracted with ethyl acetate (2×20 mL). The combined organic layers were washed with brine (50 mL), dried over anhydrous sodium sulfate, filtered and concentrated under vacuum to afford 2-(4-cyclobutylphenyl)acetic acid (120 mg, crude) as a white solid. ¹H NMR (400 MHz, CDCl₃) δ=7.42-7.17 (m, 4H), 3.63 (s, 2H), 3.60-3.47 (m, 1H), 2.41-2.28 (m, 2H), 2.20-1.96 (m, 4H), 1.92-1.79 (m, 1H).

Step 5. A mixture of 2-(4-cyclobutylphenyl)acetic acid (60.0 mg, 315 μmol, 1.00 eq), 3-(4-(3-aminoazetidin-1-yl)-2,6-difluorophenyl)piperidine-2,6-dione IX (93.1 mg, 315 μmol, 1.00 eq), 2,4,6-tripropyl-1,3,5,2,4,6-trioxatriphosphinane 2,4,6-trioxide (602 mg, 946 μmol, 50% purity in ethyl acetate, 3.00 eq) and N,N-diisopropylethylamine (204 mg, 1.58 mmol, 5.00 eq) in dimethylformamide (3 mL) was degassed under vacuum and purged 3 times with nitrogen. After stirring at 25° C. for 2 h, the mixture was diluted with water (20 mL) and extracted with ethyl acetate (2×20 mL). The combined organic layers were washed with brine (20 mL), dried over anhydrous sodium sulfate, filtered and concentrated under vacuum to give a brown residue. The residue was purified by Prep-HPLC (column: Phenomenex Synergi C18 150*25 mm*10 μm; mobile phase: [water (formic acid)-acetonitrile]; B %: 43%-73%, 10 min) and lyophilized to afford 2-(4-cyclobutylphenyl)-N-(1-(4-(2,6-dioxopiperidin-3-yl)-3,5-difluorophenyl)azetidin-3-yl)acetamide #20 (58.79 mg, 123 μmol, 39% yield, 98% purity) as a white solid. ¹H NMR (400 MHz, DMSO-d₆) δ=10.88 (s, 1H), 8.74 (d, J=6.8 Hz, 1H), 7.29-7.08 (m, 4H), 6.18 (d, J=11.2 Hz, 2H), 4.63-4.48 (m, 1H), 4.11 (t, J=7.2 Hz, 2H), 4.08-3.99 (m, 1H), 3.67-3.59 (m, 2H), 3.56-3.43 (m, 1H), 3.38 (s, 2H), 2.85-2.73 (m, 1H), 2.49-2.44 (m, 1H), 2.32-2.21 (m, 2H), 2.13-2.00 (m, 3H), 2.00-1.88 (m, 2H), 1.86-1.75 (m, 1H). MS (ESI) m/z 468.0 [M+H]⁺.

### Example 22. Synthesis of Compound 21

To a solution of 3-(4-(3-aminoazetidin-1-yl)-2,6-difluorophenyl)piperidine-2,6-dione IX (40.0 mg, 117 μmol, 1.00 eq, formate), 3-phenylbicyclo[1.1.1]pentane-1-carboxylic acid (24.3 mg, 129 μmol, 1.10 eq), N,N-diisopropylethylamine (61.2 μL, 352 μmol, 3.00 eq) and 1,2-diphenylhydrazine (15.8 mg, 117 umol, 1.00 eq) in dimethylformamide (1 mL) was added 1-(3-dimethylaminopropyl)-3-ethylcarbodiimide hydrochloride (29.2 mg, 152 μmol, 1.30 eq) at 0° C. The mixture was stirred at 20° C. for 1 h. The mixture was filtered. The filtrate was purified by prep-HPLC (column: Phenomenex Synergi C18 150*25 mm*10 μm; mobile phase: [water (formic acid)-acetonitrile]; B %: 35%-65%, 10 min) and lyophilized to afford N-(1-(4-(2,6-dioxopiperidin-3-yl)-3,5-difluorophenyl)azetidin-3-yl)-3-phenylbicyclo [1.1.1]pentane-1-carboxamide #21 (17.71 mg, 36.14 μmol, 31% yield, 95% purity) as a white solid. ¹H NMR (400 MHz, DMSO-d₆) δ=10.86 (s, 1H), 8.44 (d, J=7.3 Hz, 1H), 7.35-7.28 (m, 2H), 7.28-7.20 (m, 3H), 6.19 (s, 1H), 6.16 (s, 1H), 4.68-4.58 (m, 1H), 4.12 (t, J=7.9 Hz, 2H), 4.04 (dd, J=5.1, 12.3 Hz, 1H), 3.77-3.66 (m, 2H), 2.84-2.73 (m, 1H), 2.54 (br s, 1H), 2.18 (s, 6H), 2.12-2.02 (m, 1H), 1.98-1.90 (m, 1H). MS (ESI) m/z 466.3 [M+H]⁺.

### Example 23. Synthesis of Compound 22

Step 1. A mixture of 2-bromo-5-chloro-4-methyl-pyridine (0.500 g, 2.42 mmol, 1.00 eq), diethyl propanedioate (731 μL, 4.84 mmol, 2.00 eq), copper (I) iodide (46.1 mg, 0.242 mmol, 0.100 eq), cesium carbonate (1.58 g, 4.84 mmol, 2.00 eq) and L-proline (55.7 mg, 0.484 mmol, 0.200 eq) in dioxane (10 mL) was degassed under vacuum and purged with nitrogen for 3 times. After stirring at 110° C. for 16 h under nitrogen atmosphere, the mixture was poured into water (30 mL) and extracted with ethyl acetate (2×50 mL). The combined organic layers were washed with brine (30 mL), dried over anhydrous sodium sulfate, filtered and concentrated under vacuum to give a residue. The residue was purified by flash silica gel chromatography (20 g, ethyl acetate/petroleum ether=0/1 to 1/4) to afford diethyl 2-(5-chloro-4-methylpyridin-2-yl)malonate (300 mg, 0.997 mmol, 21% yield, 95% purity) as a yellow oil. ¹H NMR (400 MHz, CDCl₃) δ=8.44 (s, 1H), 7.18 (s, 1H), 4.23-4.18 (m, 4H), 3.37 (s, 1H), 2.38 (s, 3H), 1.30-1.26 (m, 6H).

Step 2. To a solution of diethyl diethyl 2-(5-chloro-4-methylpyridin-2-yl)malonate (300 mg, 0.997 mmol, 95% purity, 1.00 eq) in methanol (3 mL) and water (3 mL) was added lithium hydroxide monohydrate (252 mg, 6.01 mmol, 6.02 eq) at 25° C. After stirring at 25° C. for 16 h, the pH of the reaction mixture was adjusted to 3 with hydrochloric acid (1 M) and extracted with ethyl acetate (2×20 mL). The combined organic layers were washed with brine (15 mL), dried over anhydrous sodium sulfate, filtered and concentrated under vacuum to give a residue. The residue was purified by reversed-phase flash (column: Phenomenex Synergi C18; mobile phase: [water (0.225% formic acid)-acetonitrile]; B %: 0%-10%, 6 min) to afford 2-(5-chloro-4-methyl-2-pyridyl)acetic acid (200 mg, 0.969 mmol, 97% yield, 90% purity) as a white solid. ¹H NMR (400 MHz, CDCl₃) δ=8.46 (s, 1H), 7.20 (s, 1H), 3.88 (s, 2H), 2.45 (s, 3H).

Step 3. To a solution of 2-(5-chloro-4-methylpyridin-2-yl)acetic acid (68.0 mg, 329 μmol, 90% purity, 1.22 eq) and 3-3-(4-(3-aminoazetidin-1-yl)-2,6-difluorophenyl)piperidine-2,6-dione IX (80.0 mg, 270 μmol, 1.00 eq) in N,N-dimethyformamide (2 mL) was added N,N-diisopropylethylamine (141 μL, 812 umol, 3.00 eq) and 1-propanephosphonic anhydride (241 μL, 406 μmol, 50.0% purity in ethyl acetate, 1.50 eq). After stirring at 25° C. for 2 h, the mixture was poured into saturated ammonium chloride (10 mL) and extracted with ethyl acetate (2×20 mL). The combined organic phase was washed with brine (15 mL), dried over anhydrous sodium sulfate, filtered and concentrated under vacuum to give a residue. The residue was purified by Prep-HPLC (column: Unisil 3-100 C18 Ultra 150*50 mm*3 μm; mobile phase: [water (0.225% formic acid)-acetonitrile]; B %: 27%-57%, 10 min) to afford 2-(5-chloro-4-methylpyridin-2-yl)-N-(1-(4-(2,6-dioxopiperidin-3-yl)-3,5-difluorophenyl)azetidin-3-yl)acetamide #22 (56.23 mg, 118 umol, 44% yield, 98% purity) as an off-white solid. ¹H NMR (400 MHz, DMSO-d₆) δ=10.86 (s, 1H), 8.80 (d, J=6.8 Hz, 1H), 8.44 (s, 1H), 7.34 (s, 1H), 6.17 (d, J=10.8 Hz, 2H), 4.63-4.51 (m, 1H), 4.11 (t, J=7.6 Hz, 2H), 4.07-4.00 (m, 1H), 3.68-3.62 (m, 2H), 3.60 (s, 2H), 2.83-2.72 (m, 1H), 2.49-2.46 (m, 1H), 2.34 (s, 3H), 2.14-2.05 (m, 1H), 1.99-1.90 (m, 1H). MS (ESI) m/z 463.0 [M+H]⁺.

### Example 24. Synthesis of Compound 23

To a solution of 3-(4-(3-aminoazetidin-1-yl)-2,6-difluorophenyl)piperidine-2,6-dione IX (60.0 mg, 176 μmol, 1.00 eq, formate) and 3-(tert-butyl)bicyclo[1.1.1]pentane-1-carboxylic acid (44.4 mg, 264 μmol, 1.50 eq) in dimethylformamide (1 mL) was added N,N-diisopropylethylamine (68.2 mg, 527 umol, 91.9 uL, 3.00 eq). The mixture was stirred at 20° C. for 30 min. Then the 1,2-diphenylhydrazine (23.8 mg, 176 μmol, 1.00 eq) and 1-(3-dimethylaminopropyl)-3-ethylcarbodiimide hydrochloride (43.8 mg, 229 μmol, 1.30 eq) was added into the mixture at 0° C. and it was stirred at 20° C. for 1.5 h. The mixture was filtered. The filtrate was purified by prep-HPLC (column: Phenomenex luna C18 150*25 mm*10 μm; mobile phase: [water (0.225% formic acid)-acetonitrile]; B %: 41%-71%, 10 min) and lyophilized to afford 3-(tert-butyl)-N-(1-(4-(2,6-dioxopiperidin-3-yl)-3,5-difluorophenyl)azetidin-3-yl) bicyclo[1.1.1]pentane-1-carboxamide #23 (43.27 mg, 94.2 μmol, 54% yield, 97% purity) as a white solid. ¹H NMR (400 MHz, DMSO-d₆) δ=10.85 (s, 1H), 8.27 (d, J=7.3 Hz, 1H), 6.17 (s, 1H), 6.14 (s, 1H), 4.64-4.53 (m, 1H), 4.09 (t, J=7.6 Hz, 2H), 4.03 (br dd, J=5.1, 12.6 Hz, 1H), 3.67 (br t, J=6.6 Hz, 2H), 2.83-2.72 (m, 1H), 2.54-2.52 (m, 1H), 2.13-2.02 (m, 1H), 1.99-1.90 (m, 1H), 1.69 (s, 6H), 0.81 (s, 9H). MS (ESI) m/z 446.3 [M+H]⁺.

### Example 25. Synthesis of Compound 24

Step 1. A mixture of 6-bromo-2,3-dihydrobenzofuran (600 mg, 3.01 mmol, 1.00 eq), (3-methoxy-3-oxo-propanoyl)oxypotassium (37.9 μL, 4.52 mmol, 1.50 eq), (R)-(+)-2,2′-bis(diphenylphosphino)-1,1′-binaphthyl (112 mg, 180 μmol, 0.06 eq), allyl(chloro)palladium (11.0 mg, 0.0603 mmol, 0.020 eq) and 6-dimethylaminopurine (36.8 mg, 0.301 mmol, 0.100 eq) in mesitylene (6 mL) was degassed under vacuum and purged 3 times with nitrogen, and then the mixture was stirred at 130° C. under nitrogen atmosphere for 16 h. The mixture was poured into water (20 mL) and extracted with ethyl acetate (2×25 mL). The combined organic phase was washed with brine (2×15 mL), dried over anhydrous sodium sulfate, filtered and concentrated to give a residue. The residue was purified by flash silica gel chromatography (ISCO®; 20 g SepaFlash® silica flash column, eluent of 0-20% ethyl acetate/petroleum ether gradient at 30 mL/min) to afford methyl 2-(2,3-dihydrobenzofuran-6-yl)acetate (350 mg, 1.73 mmol, 57% yield, 95% purity) as a yellow oil. ¹H NMR (400 MHz, CDCl₃) δ=7.13 (d, J=7.2 Hz, 1H), 6.77-6.74 (m, 1H), 6.72 (s, 1H), 4.57 (t, J=8.4 Hz, 2H), 3.69 (s, 3H), 3.57 (s, 2H), 3.19 (t, J=8.4 Hz, 2H).

Step 2. To a solution of methyl 2-(2,3-dihydrobenzofuran-6-yl)acetate (350 mg, 1.73 mmol, 95% purity, 1.00 eq) in methanol (3 mL) and water (3 mL) was added lithium hydroxide monohydrate (363 mg, 8.65 mmol, 5.00 eq) at 25° C. After addition, the mixture was stirred at 25° C. for 2 h. The reaction mixture was adjusted pH=3 with hydrochloric acid (1 M) and extracted with ethyl acetate (2×20 mL). The combined organic layers were washed with brine (2×15 mL), dried over anhydrous sodium sulfate, filtered and concentrated under reduced pressure to afford 2-(2,3-dihydrobenzofuran-6-yl)acetic acid (260 mg, 1.39 mmol, 80.1% yield, 95% purity) as a yellow solid. ¹H NMR (400 MHz, CDCl₃) δ=7.15 (d, J=7.6 Hz, 1H), 6.77-6.74 (m, 1H), 6.73 (s, 1H), 4.58 (t, J=8.4 Hz, 2H), 3.60 (s, 2H), 3.19 (t, J=8.4 Hz, 2H).

Step 3. To a solution of 2-(2,3-dihydrobenzofuran-6-yl)acetic acid (50.0 mg, 266 μmol, 95% purity, 1.00 eq) and 3-(4-(3-aminoazetidin-1-yl)-2,6-difluorophenyl)piperidine-2,6-dione IX (75.0 mg, 254 μmol, 1.00 eq) in N,N-dimethyformamide (1 mL) was added N,N-diisopropylethylamine (139 μL, 799 umol, 3.00 eq) and 1-propanephosphonic anhydride (237 uL, 399 μmol, 50% purity in ethyl acetate, 1.50 eq). After addition, the mixture was stirred at 25° C. for 2 h. The mixture was poured into saturated ammonium chloride (10 mL) and extracted with ethyl acetate (2×20 mL). The combined organic phase was washed with brine (2×15 mL), dried over anhydrous sodium sulfate, filtered and concentrated under vacuum. The residue was purified by prep-HPLC (column: Unisil 3-100 C18 Ultra 150*50 mm*3 μm; mobile phase: [water (0.225% formic acid)-acetonitrile]; B %: 27%-57%, 10 min) to afford 2-(2,3-dihydrobenzofuran-6-yl)-N-(1-(4-(2,6-dioxopiperidin-3-yl)-3,5-difluorophenyl)azetidin-3-yl)acetamide #24 (29.60 mg, 64.4 μmol, 24% yield, 99% purity) as a white solid. ¹H NMR (400 MHz, DMSO-d₆) δ=10.87 (s, 1H), 8.82 (d, J=6.8 Hz, 1H), 7.11 (d, J=7.2 Hz, 1H), 6.70-6.68 (m, 1H), 6.65 (s, 1H), 6.16 (d, J=10.8 Hz, 2H), 4.57-4.45 (m, 3H), 4.13-4.00 (m, 3H), 3.63-3.57 (m, 2H), 3.34-3.32 (m, 2H), 3.11 (t, J=8.8 Hz, 2H), 2.80-2.71 (m, 1H), 2.46-2.43 (m, 1H), 2.13-2.00 (m, 1H), 1.95-1.85 (m, 1H). MS (ESI) m/z 456.2 [M+H]⁺.

### Example 26. Synthesis of Compound 25

Step 1. A mixture of 6-bromo-3-chloro-2-methyl-pyridine (1.00 g, 4.84 mmol, 1.00 eq), diethyl malonate (878 μL, 5.81 mmol, 1.20 eq), copper (I) iodide (92.2 mg, 0.484 mmol, 0.100 eq), cesium carbonate (4.73 g, 14.5 mmol, 3.00 eq) and (2S)-pyrrolidine-2-carboxamide (111 mg, 968 umol, 0.200 eq) in dioxane (20 mL) was degassed under vacuum and purged 3 times with nitrogen. The mixture was stirred at 110° C. for 16 h under nitrogen atmosphere. The mixture was poured into water (30 mL) and extracted with ethyl acetate (2×50 mL). The combined organic phase was washed with brine (2×30 mL), dried over anhydrous sodium sulfate, filtered and concentrated to give a residue. The residue was purified by flash silica gel chromatography (20.0 g, ethyl acetate in petroleum ether was 0%-20%) to afford diethyl 2-(5-chloro-6-methylpyridin-2-yl)malonate (1.20 g, 1.39 mmol, 29% yield, 33% purity) as a yellow oil. ¹H NMR (400 MHz, CDCl₃) δ=7.64 (d, J=8.4 Hz, 1H), 7.31 (d, J=8.4 Hz, 1H), 4.88 (s, 1H), 4.26-4.23 (m, 4H), 2.60 (s, 3H), 1.46-1.43 (m, 6H).

Step 2. To a solution of diethyl 2-(5-chloro-6-methyl-2-pyridyl)propanedioate (1.50 g, 1.73 mmol, 33% purity, 1.00 eq) in methanol (10 mL) and water (10 mL) was added lithium hydroxide monohydrate (436 mg, 10.4 mmol, 6.00 eq) at 25° C., the mixture was stirred at 25° C. for 16 h. The reaction mixture was adjusted pH=3 with hydrochloric acid (1.00 mol/L) and extracted with ethyl acetate (2×20 mL). The combined organic layers were washed with brine (15 mL), dried over anhydrous sodium sulfate, filtered and concentrated under reduced pressure to give a residue. The residue was purified by reversed-phase flash (column: Phenomenex Synergi C18; mobile phase: [water (0.225% formic acid)-acetonitrile]; B %: 0%-10%, 6 min) to afford 2-(5-chloro-6-methyl-2-pyridyl)acetic acid (150 mg, 0.800 mmol, 90% yield, 99% purity) as a white solid. ¹H NMR (400 MHz, CDCl₃) δ=7.73 (d, J=8.4 Hz, 1H), 7.08 (d, J=8.4 Hz, 1H), 3.87 (s, 2H), 2.68 (s, 3H).

Step 3. To a solution of 3-(4-(3-aminoazetidin-1-yl)-2,6-difluorophenyl)piperidine-2,6-dione IX (50.0 mg, 146 μmol, 1.00 eq) and 2-(3,5-dimethylphenyl)acetic acid (36.1 mg, 220 μmol, 1.50 eq) in N,N-dimethyformamide (1 mL) was added N,N-diisopropylethylamine (76.6 μL, 439 μmol, 3.00 eq) and 1-propanephosphonic anhydride (242 μL, 406 μmol, 50% purity in ethyl acetate, 1.50 eq), the mixture was stirred at 25° C. for 2 h. The mixture was poured into saturated ammonium chloride (10 mL) and extracted with ethyl acetate (2×20 mL). The combined organic phase was washed with brine (2×15 mL), dried over anhydrous sodium sulfate, filtered and concentrated under vacuum to give a residue. The residue was purified by prep-HPLC (column: Unisil 3-100 C18 Ultra 150*50 mm*3 μm; mobile phase: [water (0.225% formic acid)-acetonitrile]; B %: 27%-57%, 10 min) and then lyophilized to afford 2-(5-chloro-6-methylpyridin-2-yl)-N-(1-(4-(2,6-dioxopiperidin-3-yl)-3,5-difluorophenyl)azetidin-3-yl)acetamide #25 (26.6 mg, 56.5 μmol, 21% yield, 98% purity) as a white solid. ¹H NMR (400 MHz, DMSO-d₆) δ=10.86 (s, 1H), 8.69 (d, J=6.8 Hz, 1H), 7.79 (d, J=8.4 Hz, 1H), 7.21 (d, J=8.4 Hz, 1H), 6.18 (d, J=10.8 Hz, 2H), 4.61-4.50 (m, 1H), 4.11 (t, J=8.0 Hz, 2H), 4.03 (dd, J=4.8, 12.8 Hz, 1H), 3.67-3.62 (m, 2H), 3.60 (s, 2H), 2.84-2.71 (m, 1H), 2.51 (s, 3H), 2.48-2.44 (m, 1H), 2.14-2.00 (m, 1H), 1.98-1.88 (m, 1H). MS (ESI) m/z 463.0 [M+H]⁺.

### Example 27. Synthesis of Compound 26

Step 1. A mixture of 3-bromo-5-fluoro-phenol (3.00 g, 15.7 mmol, 1.00 eq), (2-chloro-2,2-difluoro-acetyl)oxysodium (5.99 g, 39.3 mmol, 2.50 eq) and cesium carbonate (7.68 g, 23.6 mmol, 1.50 eq) in N,N-dimethylformamide (50 mL) was stirred at 100° C. for 12 h. The mixture was poured into water (200 mL) and extracted with ethyl acetate (3×50 mL). The organic phase was washed with saturated calcium chloride solution (50 mL), brine (50 mL), dried over anhydrous sodium sulfate, filtered and concentrated under reduced pressure to give a residue. The residue was purified by silica gel column chromatography (petroleum ether) to give 1-bromo-3-(difluoromethoxy)-5-fluoro benzene (1.00 g, 3.73 mmol, 24% yield, 90% purity) as a colorless oil. ¹H NMR (400 MHz, CDCl₃) δ=7.16-7.10 (m, 2H), 6.87-6.82 (m, 1H), 6.70-6.33 (m, 1H).

Step 2. A mixture of 1-bromo-3-(difluoromethoxy)-5-fluorobenzene (800 mg, 3.15 mmol, 90% purity, 1.00 eq), (3-methoxy-3-oxo-propanoyl)oxypotassium (985 mg, 6.31 mmol, 2.00 eq), allyl(chloro)palladium (57.7 mg, 0.315 mmol, 0.100 eq), 2,2′-bis(diphenylphosphino)-1,1′-binaphthalene (393 mg, 0,631 mmol, 0.200 eq) and 4-dimethylaminopyridine (77.1 mg, 0.631 mmol, 0.200 eq) in 1,3,5-trimethylbenzene (10 mL) was degassed and purged 3 times with nitrogen. The mixture was stirred at 130° C. for 12 h under nitrogen atmosphere. After filtration, the filtrate was concentrated under vacuum to give a residue. The residue was purified by silica gel column chromatography (100-200 mesh, petroleum ether, then petroleum ether/ethyl acetate=9/1) to afford methyl 2-(3-(difluoromethoxy)-5-fluorophenyl)acetate (240 mg, 0.922 mmol, 29% yield, 90% purity) as a light yellow oil. ¹H NMR (400 MHz, CDCl₃) δ=6.92-6.84 (m, 2H), 6.82-6.77 (m, 1H), 6.71-6.33 (m, 1H), 3.73 (s, 3H), 3.63 (s, 2H).

Step 3. To a solution of methyl 2-(3-(difluoromethoxy)-5-fluorophenyl)acetate (227 mg, 0.871 mmol, 90% purity, 1.00 eq) in methanol (5 mL) was added the solution of lithium hydroxide monohydrate (110 mg, 2.61 mmol, 3.00 eq) in water (2 mL). After addition, the mixture was stirred at 25° C. for 12 h. The reaction mixture was adjusted pH to 3-4 with hydrochloric acid solution (1 M) and extracted with ethyl acetate (3×10 mL). The organic phase was washed by brine (10 mL), dried over anhydrous sodium sulfate, filtered and concentrated under reduced pressure to give 2-(3-(difluoromethoxy)-5-fluorophenyl)acetic acid (80.0 mg, 0.328 mmol, 38% yield, 90% purity) as a light yellow oil. The crude product was used into next step without purification. ¹H NMR (400 MHz, CDCl₃) δ=6.94-6.85 (m, 2H), 6.84-6.79 (m, 1H), 6.72-6.32 (m, 1H), 3.66 (s, 2H).

Step 4. To a solution of 2-(3-(difluoromethoxy)-5-fluorophenyl)acetic acid (65.0 mg, 266 μmol, 90% purity, 1.00 eq) and 3-(4-(3-aminoazetidin-1-yl)-2,6-difluorophenyl)piperidine-2,6-dione IX (87.2 mg, 266 μmol, 90% purity, 1.00 eq) in N,N-dimethylformamide (3 mL) was added 1-propylphosphonic anhydride (127 mg, 399 μmol, 50% purity in ethyl acetate, 1.50 eq) and N,N-diisopropylethylamine (92.6 μL, 532 μmol, 2.00 eq). The mixture was stirred at 25° C. for 2 h. The mixture was poured into water (20 mL) and extracted with ethyl acetate (3×10 mL). The organic phase was washed with saturated calcium chloride solution (20 mL), brine (20 mL), dried over anhydrous sodium sulfate, filtered and concentrated under reduced pressure to give a residue. The residue was purified by prep-HPLC (column: Phenomenex Gemini-NX C18 75*30 mm*3 μm; mobile phase: [water (0.225% formic acid)-acetonitrile]; B %: 35%-65%, 7 min) and then lyophilized to afford 2-(3-(difluoromethoxy)-5-fluorophenyl)-N-(1-(4-(2,6-dioxopiperidin-3-yl)-3,5-difluorophenyl) azetidin-3-yl)acetamide #26 (36.53 mg, 72.0 μmol, 27% yield, 98% purity) as a white solid. ¹H NMR (400 MHz, DMSO-d₆) δ=10.86 (s, 1H), 8.81 (d, J=7.2 Hz, 1H), 7.45-7.06 (m, 1H), 7.00 (dd, J=2.0, 10.0 Hz, 2H), 6.94 (s, 1H), 6.17 (d, J=10.8 Hz, 2H), 4.60-4.49 (m, 1H), 4.10 (t, J=8.0 Hz, 2H), 4.04-4.00 (m, 1H), 3.66-3.61 (m, 2H), 3.48 (s, 2H), 2.83-2.71 (m, 1H), 2.48-2.44 (m, 1H), 2.13-2.00 (m, 1H), 1.98-1.88 (m, 1H). MS (ESI) m/z 498.0 [M+H]⁺.

### Example 28. Synthesis of Compound 27

To a mixture of 3-(4-(3-aminoazetidin-1-yl)-2,6-difluorophenyl)piperidine-2,6-dione IX (50.0 mg, 169 μmol, 1.00 eq) and 2-(4-chloro-2-methoxyphenyl)acetic acid (34.0 mg, 169 μmol, 1.00 eq) in dimethylformamide (1 mL) was added N,N-diisopropylethylamine (88.5 μL, 508 μmol, 3.00 eq) at 0° C. After 1 h, 1,2-diphenylhydrazine (22.9 mg, 169 μmol, 1.00 eq) and 1-(3-dimethylaminopropyl)-3-ethylcarbodiimide hydrochlorid (42.2 mg, 220 μmol, 1.30 eq) was added, then the mixture was stirred at 25° C. for 12 h. The reaction mixture was filtered. The filtrate was purified by prep-HPLC (formic acid condition; column: Unisil 3-100 C18 Ultra 150*50 mm*3 μm; mobile phase: [water (0.225% formic acid)-acetonitrile]; B %: 35%-65%, 10 min) and lyophilized to afford a crude product. The crude product was purified by prep-HPLC (neutral condition; column: Waters Xbridge 150*25 mm*5 μm; mobile phase: [water (10 mM ammonium bicarbonate)-acetonitrile]; B %: 34%-64%, 10 min) and lyophilized to give a residue. The residue was purified by prep-HPLC (column: Waters Xbridge 150*25 mm*5 μm; mobile phase: [water (10 mM ammonium bicarbonate)-acetonitrile]; B %: 35%-65%, 8 min) and lyophilized to give a crude product. The crude product was further purified by prep-HPLC (column: Unisil 3-100 C18 Ultra 150*50 mm*3 μm; mobile phase: [water (0.225% formic acid)-acetonitrile]; B %: 33%-63%, 10 min) to afford 2-(4-chloro-2-methoxyphenyl)-N-(1-(4-(2,6-dioxopiperidin-3-yl)-3,5-difluorophenyl) azetidin-3-yl)acetamide #27 (7.82 mg, 16.20 μmol, 26% yield, 99% purity) as a white solid. ¹H NMR (400 MHz, DMSO-d₆) δ=10.88 (br s, 1H), 8.62 (br d, J=7.0 Hz, 1H), 8.44 (br s, 1H), 7.18 (d, J=8.2 Hz, 1H), 7.03 (d, J=1.6 Hz, 1H), 6.95 (dd, J=1.8, 8.0 Hz, 1H), 6.20 (s, 1H), 6.17 (s, 1H), 4.60-4.53 (m, 1H), 4.11 (br t, J=7.8 Hz, 2H), 4.04 (br dd, J=5.0, 12.8 Hz, 1H), 3.78 (s, 3H), 3.64 (br dd, J=6.2, 7.0 Hz, 3H), 3.40 (br s, 1H), 2.83-2.73 (m, 2H), 2.10-2.06 (m, 1H), 1.97-1.92 (m, 1H). MS (ESI) m/z 478.1 [M+H]⁺.

### Example 29. Synthesis of Compound 28

To a solution of 2-(3-(trifluoromethoxy)phenyl)acetic acid (49.9 mg, 227 μmol, 1.20 eq) in N,N-dimethyformamide (4 mL) was added 3-(4-(3-aminoazetidin-1-yl)-2,6-difluorophenyl) piperidine-2,6-dione (108 mg, 283 μmol, 1.50 eq) and N,N-diisopropylethylamine (65.8 μL, 378 μmol, 2.00 eq). After stirring for 10 min, 3-(4-(3-aminoazetidin-1-yl)-2,6-difluorophenyl) piperidine-2,6-dione IX (62.0 mg, 189 μmol, 90% purity, 1.00 eq) was added. The mixture was stirred at 25° C. for 12 h. The mixture was poured into water (30 mL) and extracted with ethyl acetate (3×10 mL). The organic phase was washed with brine (20 mL), dried over anhydrous sodium sulfate, filtered and concentrated under reduced pressure to give a residue. The residue was purified by prep-HPLC (column: Phenomenex Synergi C18 150*25 mm*10 μm; mobile phase: [water (0.225% formic acid)-acetonitrile]; B %: 39%-69%, 10 min) and lyophilized to afford N-(1-(4-(2,6-dioxopiperidin-3-yl)-3,5-difluorophenyl)azetidin-3-yl)-2-(3-(trifluoromethoxy)phenyl) acetamide #28 (39.76 mg, 79.13 μmol, 42% yield, 99% purity) as an off-white solid. ¹H NMR (400 MHz, DMSO-d₆) δ=10.88 (s, 1H), 8.84 (d, J=7.2 Hz, 1H), 7.53-7.39 (m, 1H), 7.35-7.19 (m, 3H), 6.18 (d, J=10.8 Hz, 2H), 4.62-4.46 (m, 1H), 4.11 (t, J=7.6 Hz, 2H), 4.04 (dd, J=4.8, 12.0 Hz, 1H), 3.63 (dd, J=5.6, 8.0 Hz, 2H), 3.51 (s, 2H), 2.85-2.71 (m, 1H), 2.48-2.44 (m, 1H), 2.15-2.00 (m, 1H), 1.98-1.88 (m, 1H). MS (ESI) m/z 498.0 [M+H]⁺.

### Example 30. Synthesis of Compound 29

To a solution of 3-(4-(3-aminoazetidin-1-yl)-2,6-difluorophenyl)piperidine-2,6-dione IX (50.0 mg, 146 μmol, 1.00 eq, formic acid) and 2-(3,5-dimethylphenyl)acetic acid (36.1 mg, 220 μmol, 1.50 eq) in dimethyformamide (1 mL) was added N,N-diisopropylethylamine (56.8 mg, 439 μmol, 76.6 uL, 3.00 eq). The mixture was stirred at 20° C. for 30 min. Then 1,2-diphenylhydrazine (19.8 mg, 146 μmol, 1.00 eq) and 1-(3-dimethylaminopropyl)-3-ethylcarbodiimide hydrochloride (36.5 mg, 190 μmol, 1.30 eq) was added into the mixture at 0° C. and it was stirred at 20° C. for 1.5 h. The mixture was filtered. The filtrate was purified by Prep-HPLC (column: Phenomenex Synergi C18 150*25 mm*10 μm; mobile phase: [water (0.225% formic acid)-acetonitrile]; B %: 35%-65%, 10 min) and lyophilized to afford 2-(3,5-dimethylphenyl)-N-(1-(4-(2,6-dioxopiperidin-3-yl)-3,5-difluorophenyl)azetidin-3-yl)acetamide #29 (22.77 mg, 50.6 μmol, 35% yield, 98% purity) as a white solid. ¹H NMR (400 MHz, DMSO-d₆) δ=10.85 (s, 1H), 8.70 (d, J=6.97 Hz, 1H), 6.74-6.95 (m, 3H), 6.17 (d, J=11.13 Hz, 2H), 4.49-4.58 (m, 1H), 4.10 (t, J=7.70 Hz, 2H), 4.03 (br dd, J=12.53, 4.95 Hz, 1H), 3.62 (dd, J=7.76, 5.56 Hz, 2H), 3.33-3.35 (m, 2H), 2.69-2.84 (m, 1H), 2.51-2.54 (m, 1H), 2.23 (s, 6H), 2.03-2.12 (m, 1H), 1.87-1.99 (m, 1H). MS (ESI) m/z 442.2 [M+H]⁺.

### Example 31. Synthesis of Compound 30

To a mixture of 3-(4-(3-aminoazetidin-1-yl)-2,6-difluorophenyl)piperidine-2,6-dione IX (50.0 mg, 169 μmol, 1.00 eq) and 2-(4-fluoro-3-methylphenyl)acetic acid (28.5 mg, 169 μmol, 1.00 eq) in dimethylformamide (1 mL) was added N-ethyl-N,N-diisopropylamine (65.7 mg, 508 μmol, 88.5 μL, 3.00 eq) at 0° C. After 1 h, benzotriazol-1-ol (22.9 mg, 169 μmol, 1.00 eq) and 1-(3-dimethylaminopropyl)-3-ethylcarbodiimide hydrochloride (42.2 mg, 220 μmol, 1.30 eq) was added, then the mixture was stirred at 25° C. for 12 h. The reaction mixture was filtered. The filtrate was purified by Prep-HPLC (column: Unisil 3-100 C18 Ultra 150*50 mm*3 μm; mobile phase: [water (0.225% formic acid)-acetonitrile]; B %: 32%-62%, 10 min) to afford N-(1-(4-(2,6-dioxopiperidin-3-yl)-3,5-difluorophenyl) azetidin-3-yl)-2-(4-fluoro-3-methylphenyl)acetamide #30 (30.9 mg, 68.0 μmol, 40% yield, 98% purity) as a white solid. ¹H NMR (400 MHz, DMSO-d₆) δ=10.87 (s, 1H), 8.74 (br d, J=6.8 Hz, 1H), 7.15 (br d, J=6.6 Hz, 1H), 7.12-7.01 (m, 2H), 6.18 (br d, J=11.0 Hz, 2H), 4.61-4.48 (m, 1H), 4.11 (br t, J=7.6 Hz, 2H), 4.04 (br dd, J=4.8, 12.8 Hz, 1H), 3.63 (br dd, J=5.6, 7.5 Hz, 2H), 3.38 (s, 2H), 2.84-2.71 (m, 1H), 2.48-2.45 (m, 1H), 2.21 (s, 3H), 2.13-2.02 (m, 1H), 1.99-1.89 (m, 1H). MS (ESI) m/z 446.1 [M+H]⁺.

### Example 32. Synthesis of Compound 31

To a mixture of 3-(4-(3-aminoazetidin-1-yl)-2,6-difluorophenyl)piperidine-2,6-dione IX (50.0 mg, 169 μmol, 1.00 eq) and 2-(4-chloro-2-fluorophenyl)acetic acid (31.9 mg, 169 μmol, 1.00 eq) in dimethylformamide (1 mL) was added N,N-diisopropylethylamine (88.5 μL, 508 μmol, 3.00 eq). The mixture was stirred at 25° C. for 1 h. Then the 1,2-diphenylhydrazine (22.8 mg, 169 μmol, 1.00 eq) and 1-(3-dimethylaminopropyl)-3-ethylcarbodiimide hydrochloride (42.2 mg, 220 μmol, 1.30 eq) was added and the mixture was stirred at 25° C. for 3 h. The reaction mixture was filtered. The filtrate was purified by Prep-HPLC (formic acid condition; column: Unisil 3-100 C18 Ultra 150*50 mm*3 μm; mobile phase: [water (0.225% formic acid)-acetonitrile]; B %: 35%-65%, 10 min) to afford 2-(4-chloro-2-fluorophenyl)-N-(1-(4-(2,6-dioxopiperidin-3-yl)-3,5-difluorophenyl)azetidin-3-yl)acetamide #31 (30.0 mg, 63.8 μmol, 38% yield, 99% purity) as a white solid. ¹H NMR (400 MHz, DMSO-d₆) δ=10.87 (s, 1H), 8.81 (br d, J=6.96 Hz, 1H), 8.42 (s, 1H), 7.33-7.42 (m, 2H), 7.25 (dd, J=8.26, 2.02 Hz, 1H), 6.20 (s, 1H), 6.17 (s, 1H), 4.48-4.63 (m, 1H), 4.12 (t, J=7.70 Hz, 2H), 4.04 (br dd, J=12.36, 5.01 Hz, 1H), 3.65 (br dd, J=7.70, 5.50 Hz, 2H), 3.51 (s, 2H), 2.74-2.84 (m, 1H), 2.48-2.49 (m, 1H), 2.04-2.11 (m, 1H), 1.95 (ddd, J=7.92, 5.23, 2.51 Hz, 1H). MS (ESI) m/z 466.2 [M+H]⁺.

### Example 33. Synthesis of Compound 32

To a mixture of 3-(4-(3-aminoazetidin-1-yl)-2,6-difluoro-phenyl)piperidine-2,6-dione (30.0 mg, 102 μmol, 1.00 eq) and 2-(3-chlorophenyl)acetic acid (17.3 mg, 102 μmol, 1.00 eq) in dimethylformamide (1 mL) was added N,N-diisopropylethylamine (53.1 μL, 305 μmol, 3.00 eq). The mixture was stirred at 25° C. for 1 h. Then 1,2-diphenylhydrazine (13.7 mg, 102 μmol, 1.00 eq) and 1-(3-dimethylaminopropyl)-3-ethylcarbodiimide hydrochloride (25.3 mg, 132 μmol, 1.30 eq) was added into the mixture. It was stirred at 25° C. for 2 h. The reaction mixture was filtered. The filtrate was purified by Prep-HPLC (column: Unisil 3-100 C18 Ultra 150*50 mm*3 μm; mobile phase: [water (0.225% formic acid)-acetonitrile]; B %: 35%-65%, 10 min) to afford 2-(3-chlorophenyl)-N-(1-(4-(2,6-dioxo-3-piperidyl)-3,5-difluoro-phenyl)azetidin-3-yl)acetamide #32 (28.0 mg, 61.1 μmol, 60% yield, 98% purity) as a white solid. ¹H NMR (400 MHz, DMSO-d₆) δ=10.86 (s, 1H), 8.81 (d, J=6.96 Hz, 1H), 7.28-7.38 (m, 3H), 7.21-7.25 (m, 1H), 6.20 (s, 1H), 6.17 (s, 1H), 4.51-4.59 (m, 1H), 4.12 (t, J=7.70 Hz, 2H), 4.04 (dd, J=12.72, 5.01 Hz, 1H), 3.64 (dd, J=7.82, 5.62 Hz, 2H), 3.46 (s, 2H), 2.73-2.84 (m, 1H), 2.50 (m, 1H), 2.08 (qd, J=13.10, 3.85 Hz, 1H), 1.91-1.99 (m, 1H). MS (ESI) m/z 448.2 [M+H]⁺.

### Example 34. Synthesis of Compound 33

To a solution of 3-(4-(3-aminoazetidin-1-yl)-2-chlorophenyl)piperidine-2,6-dione XVI (100 mg, 340 μmol, 1.00 eq) and 2-(4-chloro-3-methylphenyl)acetic acid (75.4 mg, 408 μmol, 1.20 eq) in dimethyl formamide (1.50 mL) was added O-(7-azabenzotriazol-1-yl)-N,N,N′,N′-tetramethyluronium (194 mg, 511 μmol, 1.50 eq) and N,N-diisopropylethylamine (178 μL, 132 mg, 1.02 mmol, 3.00 eq). The mixture was stirred at 30° C. for 12 h. The pH of the mixture was adjusted to around 6 by adding formic acid. The mixture was purified by prep-HPLC (column: Phenomenex Synergi C18 150*25 mm*10 μm; mobile phase: [water (0.225% formic acid)-acetonitrile]; B %: 35%-68%, 11 min) to afford 2-(4-chloro-3-methylphenyl)-N-(1-(3-chloro-4-(2,6-dioxopiperidin-3-yl) phenyl)azetidin-3-yl)acetamide #33 (42.6 mg, 91.6 μmol, 27% yield, 99% purity) as a white solid. ¹H NMR (400 MHz, DMSO-d₆) δ=10.81 (s, 1H), 8.74 (d, J=7.0 Hz, 1H), 7.32 (d, J=8.2 Hz, 1H), 7.21 (s, 1H), 7.12-7.06 (m, 2H), 6.49 (d, J=2.4 Hz, 1H), 6.40 (dd, J=2.3, 8.4 Hz, 1H), 4.59-4.50 (m, 1H), 4.10 (t, J=7.5 Hz, 2H), 4.03 (dd, J=5.0, 12.1 Hz, 1H), 3.60 (dd, J=5.6, 7.6 Hz, 2H), 3.39 (br s, 2H), 2.78-2.66 (m, 2H), 2.30 (s, 3H), 2.25-2.16 (m, 1H), 1.96-1.88 (m, 1H). MS (ESI) m/z 460.2 [M+H]⁺.

### Example 35. Synthesis of Compound 34

To a solution of 3-(4-(3-aminoazetidin-1-yl)-2-chlorophenyl)piperidine-2,6-dione XVI (100 mg, 340 μmol, 1.00 eq) and 2-(4-chlorophenyl)acetic acid (69.7 mg, 408 μmol, 1.20 eq) in dimethyl formamide (1.50 mL) was added O-(7-azabenzotriazol-1-yl)-N,N,N′,N′-tetramethyluronium (194 mg, 511 μmol, 1.50 eq) and N,N-diisopropylethylamine (178 μL, 1.02 mmol, 3.00 eq). The mixture was stirred at 25° C. for 12 h. The pH of the mixture was adjusted to around 6 by adding formic acid. The reaction mixture filtered and concentrated under reduced pressure to give a residue. The residue was purified by prep-HPLC (column: Phenomenex Synergi C18 150*25 mm*10 μm; mobile phase: [water (0.225% formic acid)-acetonitrile]; B %: 31%-64%, 11 min) and lyophilized to give a white solid. The white solid was re-purified by prep-HPLC (column: Phenomenex Synergi C18 150*25 mm*10 μm; mobile phase: [water (0.225% formic acid)-acetonitrile]; B %: 32%-62%, 10 min) and lyophilized to afford N-(1-(3-chloro-4-(2,6-dioxopiperidin-3-yl)phenyl)azetidin-3-yl)-2-(4-chlorophenyl)acetamide #34 (35.9 mg, 78.8 μmol, 23% yield, 98% purity) as a white solid. ¹H NMR (400 MHz, DMSO-d₆) δ=10.82 (s, 1H), 8.77 (d, J=7.0 Hz, 1H), 7.38-7.33 (m, 2H), 7.32-7.24 (m, 2H), 7.09 (d, J=8.5 Hz, 1H), 6.50 (d, J=2.4 Hz, 1H), 6.40 (dd, J=2.3, 8.3 Hz, 1H), 4.59-4.50 (m, 1H), 4.10 (t, J=7.5 Hz, 2H), 4.03 (dd, J=4.8, 12.2 Hz, 1H), 3.60 (dd, J=5.5, 7.8 Hz, 2H), 3.43 (s, 2H), 2.76-2.69 (m, 1H), 2.52 (br s, 1H), 2.24-2.16 (m, 1H), 1.96-1.89 (m, 1H). MS (ESI) m/z 446.2 [M+H]⁺.

### Example 36. Synthesis of Compound 35

To a solution of 3-(4-(3-aminoazetidin-1-yl)-2-chlorophenyl)piperidine-2,6-dione XVI (100 mg, 340 μmol, 1.00 eq) and 2-(4-chlorophenyl)-2,2-difluoroacetic acid (84.4 mg, 408 μmol, 1.20 eq) in dimethyl formamide (1.50 mL) was added O-(7-azabenzotriazol-1-yl)-N,N,N′,-tetramethyluronium (194 mg, 510 μmol, 1.50 eq) and N,N-diisopropylethylamine (178 μL, 1.02 mmol, 3.00 eq). The mixture was stirred at 25° C. for 12 h. The pH of the mixture was adjusted to around 6 by adding formic acid. The reaction mixture was filtered and the filtrate was purified by prep-HPLC (column: Phenomenex Synergi C18 150*25 mm*10 μm; mobile phase: [water (0.225% formic acid)-acetonitrile]; B %: 39%-72%, 11 min) and lyophilized to afford N-(1-(3-chloro-4-(2,6-dioxopiperidin-3-yl)phenyl)azetidin-3-yl)-2-(4-chlorophenyl)-2,2-difluoroacetamide #35 (23.9 mg, 49.2 μmol, 14% yield, 99% purity) as a white solid. ¹H NMR (400 MHz, DMSO-d₆) δ=10.80 (s, 1H), 9.61 (br d, J=6.8 Hz, 1H), 7.61 (s, 4H), 7.09 (d, J=8.4 Hz, 1H), 6.50 (d, J=2.3 Hz, 1H), 6.40 (dd, J=2.3, 8.4 Hz, 1H), 4.65 (qd, J=6.7, 13.0 Hz, 1H), 4.14 (t, J=7.6 Hz, 2H), 4.04 (dd, J=5.0, 12.2 Hz, 1H), 3.75 (dd, J=5.9, 7.6 Hz, 2H), 2.79-2.68 (m, 1H), 2.45-2.34 (m, 1H), 2.21 (dt, J=8.5, 12.6 Hz, 1H), 1.97-1.88 (m, 1H). MS (ESI) m/z 482.2 [M+H]⁺.

### Example 37. Synthesis of Compound 36

Step 1. To a solution of 2-(4-chlorophenyl)acetic acid (140 mg, 0.821 mmol, 1.00 eq) in dichloromethane (5 mL) was added oxalyl dichloride (360 μL, 4.10 mmol, 5.00 eq) and dimethyl formamide (63.1 μL, 0.821 mmol, 1.00 eq). The mixture was stirred at 25° C. for 5 h. The mixture was concentrated to give 2-(4-chlorophenyl)acetyl chloride (120 mg, crude) as a yellow oil and was used to the next step without purification.

Step 2. To a solution of 3-(4-(3-aminoazetidin-1-yl)-2,6-difluorophenyl)piperidine-2,6-dione IX (100 mg, 293 μmol, 1.00 eq, formic acid) in dimethyl formamide (2 mL) was added 2-(4-chlorophenyl)acetyl chloride (51.5 μL, 352 μmol, 1.20 eq) and triethylamine (122 μL, 876 μmol, 2.99 eq). The mixture was stirred at 25° C. for 12 h. The mixture was filtered to give filtrate. The filtrate was purified by reverse phase HPLC (column: spherical C18, 20-45 μm, 100 Å, SW 120, mobile phase: [water (0.1% formic acid)-acetonitrile). The desired fraction was collected and lyophilized to give a residue. The residue was purified by prep-NPLC (column: Welch Ultimate XB-CN 250*50*10 μm; mobile phase: [hexane-isopropyl alcohol]; B %: 25%-65%, 15 min) and further purified by prep-HPLC (column: Unisil 3-100 C18 Ultra 150*50 mm*3 μm; mobile phase: [water (0.225% formic acid)-acetonitrile]; B %: 35%-65%, 10 min). The desired fraction was collected and lyophilized to give 2-(4-chlorophenyl)-N-(1-(4-(2,6-dioxopiperidin-3-yl)-3,5-difluorophenyl)azetidin-3-yl)acetamide #36 (11.8 mg, 25.5 μmol, 8.7% yield, 97% purity) as a white solid. ¹H NMR (400 MHz, DMSO-d₆) δ=10.88 (s, 1H), 8.81 (br t, J=6.1 Hz, 1H), 7.40-7.32 (m, 2H), 7.31-7.22 (m, 2H), 6.17 (d, J=11.1 Hz, 2H), 4.59-4.48 (m, 1H), 4.10 (t, J=7.7 Hz, 2H), 4.04 (br dd, J=5.0, 12.5 Hz, 1H), 3.66-3.59 (m, 2H), 3.43 (s, 2H), 2.82-2.72 (m, 1H), 2.47 (br s, 1H), 2.13-2.02 (m, 1H), 1.98-1.89 (m, 1H). MS (ESI) m/z 448.2 [M+H]⁺.

### Example 38. Synthesis of Compound 37

To a solution of 3-(4-(3-aminoazetidin-1-yl)-2,6-difluorophenyl)piperidine-2,6-dione IX (100 mg, 339 μmol, 1.00 eq) in dimethyl formamide (1 mL) was added 2-(4-chlorophenyl)-2,2-difluoroacetic acid (70.0 mg, 339 μmol, 1.00 eq), 0-(7-azabenzotriazol-1-yl)-N,N,N′,N′-tetramethyluronium hexafluorophosphate (193 mg, 508 μmol, 1.50 eq) and triethylamine (141 μL, 1.02 mmol, 3.00 eq). The mixture was stirred at 25° C. for 3 h. The mixture was filtered to give filtrate. The filtrate was purified by prep-HPLC (column: Phenomenex luna C18 150*25 mm*10 μm; mobile phase: [water (0.2% formic acid)-acetonitrile]; B %: 45%-75%, 12 min). The desired fraction was collected and lyophilized to give 2-(4-chlorophenyl)-N-(1-(4-(2,6-dioxopiperidin-3-yl)-3,5-difluorophenyl)azetidin-3-yl)-2,2-difluoroacetamide #37 (15.8 mg, 31.6 μmol, 9% yield, 97% purity) as an off-white solid. ¹H NMR (400 MHz, DMSO-d₆) δ=10.88 (s, 1H), 9.66 (br d, J=7.0 Hz, 1H), 7.62 (s, 4H), 6.18 (d, J=11.1 Hz, 2H), 4.64 (br d, J=6.6 Hz, 1H), 4.13 (t, J=7.9 Hz, 2H), 4.04 (br dd, J=5.1, 12.7 Hz, 1H), 3.80-3.71 (m, 2H), 2.83-2.73 (m, 1H), 2.48-2.45 (m, 1H), 2.12-2.02 (m, 1H), 1.98-1.89 (m, 1H). MS (ESI) m/z 484.2 [M+H]⁺.

### Example 39. Synthesis of Compound 38

Step 1. To a mixture of 2-(1-(tert-butoxycarbonyl)azetidin-3-yl)acetic acid (1.00 g, 4.65 mmol, 1.00 eq) and N,N-diisopropylethylamine (2.43 mL, 13.9 mmol, 3.00 eq) and O-(7-azabenzotriazol-1-yl)-N,N,N,N-tetramethyluroniumhexafluorophosphate (2.12 g, 5.58 mmol, 1.20 eq) in dichloromethane (10 mL) was added 3-chloro-4-methyl-5-(morpholinomethyl)aniline (1.34 g, 5.58 mmol, 1.20 eq) (see Compound 18). The reaction mixture was stirred at 20° C. for 2 h. The reaction mixture was concentrated to give a residue. The residue was purified by column chromatography (ISCO®; 120 g SepaFlash® Silica Flash Column, Eluent of 0-100% ethyl acetate/petroleum ether at 80 mL/min) to afford tert-butyl 3-(2-((3-chloro-4-methyl-5-(morpholinomethyl)phenyl)amino)-2-oxoethyl)azetidine-1-carboxylate (2.00 g, 4.57 mmol, 98% yield) as a yellow solid. MS (ESI) m/z 438.2 [M+H]⁺.

Step 2. To a mixture of tert-butyl 3-(2-((3-chloro-4-methyl-5-(morpholinomethyl)phenyl)amino)-2-oxoethyl) azetidine-1-carboxylate (2.00 g, 4.57 mmol, 1.00 eq) in dichloromethane (50 mL) was added trifluoroacetic acid (10.0 mL, 135 mmol, 29.5 eq). The reaction mixture was stirred at 20° C. for 2 h. The reaction was concentrated to give a residue. The residue was used to next step without further purification.

Step 3. To a mixture of 3-(4-bromo-2,6-difluorophenyl)piperidine-2,6-dione V (400 mg, 1.32 mmol, 1.00 eq), 2-(azetidin-3-yl)-N-(3-chloro-4-methyl-5-(morpholinomethyl)phenyl)acetamide (577 mg, 1.71 mmol, 1.30 eq), cesium carbonate (1.29 g, 3.95 mmol, 3.00 eq) in dioxane (10 mL) was added 4,5-bis(diphenylphosphino)-9,9-dimethylxanthene (38.0 mg, 0.065.7 mmol, 0.0500 eq) and tris(dibenzylideneacetone)dipalladium(0) (60.2 mg, 0.0657 mmol, 0.0500 eq) under nitrogen atmosphere. The reaction mixture was stirred at 100° C. for 24 h. The reaction mixture was concentrated to give a residue. The residue was purified by Prep-HPLC (column: Phenomenex Synergi C18 150*25 mm*10 μm; mobile phase: [water (0.225% formic acid)-acetonitrile]; B %: 12%-42%, 10 min) to give a crude product. The crude product was re-purified by Prep-HPLC (column: Phenomenex Synergi C18 150*25 mm*10 μm; mobile phase: [water (0.225% formic acid)-acetonitrile]; B %: 10%-40%, 10 min), NPLC (column: Welch Ultimate XB-CN 250*70*10 μm; mobile phase: [hexane-ethanol]; B %: 27%-67%, 12 min) and NPLC (column: Welch Ultimate XB-SiOH 250*50*10 μm; mobile phase: [hexane-ethanol]; B %: 20%-60%, 15 min) to afford N-(3-chloro-4-methyl-5-(morpholinomethyl)phenyl)-2-(1-(4-(2,6-dioxopiperidin-3-yl)-3,5-difluorophenyl)azetidin-3-yl)acetamide #38 (10.8 mg, 0.0183 mmol, 1% yield) as a yellow solid. ¹H NMR (400 MHz, DMSO-d₆) δ=10.85 (s, 1H), 10.09 (s, 1H), 7.77 (d, J=2.0 Hz, 1H), 7.35 (d, J=2.0 Hz, 1H), 6.12 (d, J=11.0 Hz, 2H), 4.06-3.96 (m, 3H), 3.56 (br d, J=4.8 Hz, 6H), 3.41 (s, 2H), 3.10-2.99 (m, 1H), 2.83-2.66 (m, 4H), 2.36 (br s, 4H), 2.29 (s, 3H), 2.13-2.00 (m, 1H), 1.98-1.90 (m, 1H). MS (ESI) m/z 561.2 [M+H]⁺.

### Example 40. Synthesis of Compound 39

Step 1. To a mixture of 2-(1-(tert-butoxycarbonyl)azetidin-3-yl)acetic acid (0.500 g, 2.32 mmol, 1.00 eq) and N,N-diisopropylethylamine (1.21 mL, 6.97 mmol, 3.00 eq) and O-(7-azabenzotriazol-1-yl)-N,N,N,N-tetramethyluroniumhexafluorophosphate (1.06 g, 2.79 mmol, 1.20 eq) in dichloromethane (10 mL) was added 3-(trifluoromethoxy)aniline (371 μL, 2.79 mmol, 1.20 eq). The reaction mixture was stirred at 20° C. for 2 h. The reaction mixture was concentrated under reduced pressure to give a residue. The residue was purified by silica gel chromatography (petroleum ether/ethyl acetate=10/1 to 0/1) to afford tert-butyl 3-(2-oxo-2-((3-(trifluoromethoxy)phenyl)amino)ethyl)azetidine-1-carboxylate (0.800 g, 2.14 mmol, 92% yield) as a yellow solid. ¹H NMR (400 MHz, DMSO-d₆) δ=10.26 (s, 1H), 7.78 (s, 1H), 7.48-7.44 (m, 1H), 7.44-7.39 (m, 1H), 7.02 (br d, J=7.6 Hz, 1H), 3.96 (br s, 2H), 3.58 (br s, 2H), 2.91-2.81 (m, 1H), 2.69 (s, 2H), 1.37 (s, 9H).

Step 2. To a mixture of tert-butyl 3-(2-oxo-2-((3-(trifluoromethoxy)phenyl)amino)ethyl)azetidine-1-carboxylate (250 mg, 668 μmol, 1.00 eq) in dichloromethane (8 mL) was added trifluoroacetic acid (1.60 mL). The reaction mixture was stirred at 20° C. for 2 h. The mixture was diluted with toluene (1 mL) and concentrated under reduced pressure to afford 2-(azetidin-3-yl)-N-(3-(trifluoromethoxy)phenyl)acetamide (200 mg, crude) as a white solid and was used to the next step without purification. MS (ESI) m/z. 275.2 [M+H]⁺.

Step 3. A solution of 3-(4-bromo-2,6-difluorophenyl)piperidine-2,6-dione V (200 mg, 0.658 mmol, 1.00 eq) in dioxane (2 mL) was added 2-(azetidin-3-yl)-N-(3-(trifluoromethoxy)phenyl)acetamide (200 mg, 0.729 mmol, 1.11 eq), cesium carbonate (643 mg, 1.97 mmol, 3.00 eq), bis(dibenzylideneacetone)palladium (30.1 mg, 0.0329 mmol, 0.0500 eq) and 4,5-bis(diphenylphosphino)-9,9-dimethylxanthene (57.1 mg, 0.0987 mmol, 0.150 eq) under nitrogen protected, and then the mixture was stirred at 100° C. for 12 h under nitrogen atmosphere. The mixture was diluted with water (80 mL) and extracted with ethyl acetate (3×20 mL). The combined organic layers were dried over anhydrous sodium sulfate, filtered and concentrated under reduced pressure to give a residue. The residue was purified by chromatography on silica gel (petroleum ether/ethyl acetate=1/0 to 50/1) and Prep-HPLC (column: Phenomenex Synergi C18 150*25 mm*10 μm; mobile phase: [water (0.225% formic acid)-acetonitrile]; B %: 44%-74%, 10 min). The desired eluent was lyophilized and further purified by Prep-NPLC (column: Welch Ultimate XB-CN 250*50*10 μm; mobile phase: [hexane-isopropyl alcohol]; B %: 35%-75%, 15 min) to afford 2-(1-(4-(2,6-dioxopiperidin-3-yl)-3,5-difluorophenyl)azetidin-3-yl)-N-(3-(trifluoromethoxy)phenyl)acetamide #39 (27.68 mg, 49.9 μmol, 8% yield, 98% purity, formic acid) as a white solid. ¹H NMR (400 MHz, DMSO-d₆) δ=10.85 (s, 1H), 10.31 (s, 1H), 7.80 (s, 1H), 7.50-7.37 (m, 2H), 7.02 (br d, J=7.6 Hz, 1H), 6.12 (d, J=11.2 Hz, 2H), 4.07-3.94 (m, 3H), 3.56 (br t, J=6.4 Hz, 2H), 3.10-3.01 (m, 1H), 2.83-2.76 (m, 1H), 2.74 (br d, J=7.6 Hz, 2H), 2.47 (br s, 1H), 2.14-2.00 (m, 1H), 1.99-1.89 (m, 1H). MS (ESI) m/z. 498.2 [M+H]⁺.

### Example 41. Synthesis of Compound 40

Step 1. To a mixture of 2-(1-(tert-butoxycarbonyl)azetidin-3-yl)acetic acid (0.500 g, 2.32 mmol, 1.00 eq) and N,N-diisopropylethylamine (1.21 mL, 6.97 mmol, 3.00 eq) and O-(7-azabenzotriazol-1-yl)-N,N,N,N-tetramethyluroniumhexafluorophosphate (1.06 g, 2.79 mmol, 1.20 eq) in dichloromethane (10 mL) was added 3-chloro-4-methylaniline (0.394 g, 2.79 mmol, 1.20 eq). The reaction mixture was stirred at 20° C. for 2 h. The mixture was concentrated in vacuum to give a residue. The residue was purified by silica gel chromatography (petroleum ether/ethyl acetate=10/1 to 0/1) to give tert-butyl 3-(2-((3-chloro-4-methylphenyl)amino)-2-oxoethyl)azetidine-1-carboxylate (0.700 g, 2.07 mmol, 88% yield) as a yellow solid. ¹H NMR (400 MHz, DMSO-d₆) δ=10.05 (s, 1H), 7.78 (d, J=1.6 Hz, 1H), 7.35-7.30 (m, 1H), 7.29-7.23 (m, 1H), 3.96 (br s, 2H), 3.57 (br s, 2H), 2.92-2.79 (m, 1H), 2.63 (d, J=8.0 Hz, 2H), 2.26 (s, 3H), 1.38 (s, 9H). MS (ESI) m/z 283.0 [M+H-56]⁺.

Step 2. A mixture of tert-butyl 3-(2-((3-chloro-4-methylphenyl)amino)-2-oxoethyl)azetidine-1-carboxylate (500 mg, 1.48 mmol, 1.00 eq) in dichloromethane (2 mL) was added trifluoroacetic acid (0.400 mL). The reaction mixture was stirred at 20° C. for 2 h. The reaction mixture was concentrated in vacuum to give a residue. The residue was purified by reversed-phase HPLC (0.1% formic acid) to give 2-(azetidin-3-yl)-N-(3-chloro-4-methylphenyl)acetamide (200 mg, crude) as a yellow solid. MS (ESI) m/z 239.1 [M+H]⁺.

Step 3. A mixture of 3-(4-bromo-2,6-difluorophenyl)piperidine-2,6-dione V (200 mg, 0.657 mmol, 1.00 eq), 2-(azetidin-3-yl)-N-(3-chloro-4-methylphenyl)acetamide (188 mg, 0.789 mmol, 1.20 eq), cesium carbonate (642 mg, 1.97 mmol, 3.00 eq) and palladium(II) acetate (7.38 mg, 0.0328 mmol, 0.050 eq) in dioxane (2 mL) was added 4,5-bis(diphenylphosphino)-9,9-dimethylxanthene (19.0 mg, 0.0328 mmol, 0.050 eq) under nitrogen atmosphere. The reaction mixture was stirred at 100° C. for 12 h. The reaction mixture was concentrated to give a residue. The residue was purified by Prep-HPLC (column: Phenomenex Gemini-NX C18 75*30 mm*3 μm; mobile phase: [water (0.225% formic acid)-acetonitrile]; B %: 42%-72%, 7 min) and lyophilized to give N-(3-chloro-4-methylphenyl)-2-(1-(4-(2,6-dioxopiperidin-3-yl)-3,5-difluorophenyl) azetidin-3-yl)acetamide #40 (7.49 mg, 0.0146 mmol, 2% yield, 99% purity, formic acid) as a yellow solid. ¹H NMR (400 MHz, DMSO-d₆) δ=10.86 (s, 1H), 10.11 (s, 1H), 7.80 (s, 1H), 7.35 (dd, J=1.2, 8.0 Hz, 1H), 7.29-7.24 (m, 1H), 6.13 (d, J=11.2 Hz, 2H), 4.09-3.95 (m, 3H), 3.56 (br t, J=6.4 Hz, 2H), 3.04 (td, J=6.8, 13.6 Hz, 1H), 2.83-2.75 (m, 1H), 2.71 (br d, J=8.0 Hz, 2H), 2.48-2.37 (m, 1H), 2.27 (s, 3H), 2.14-2.01 (m, 1H), 1.99-1.88 (m, 1H). MS (ESI) m/z 462.0 [M+H]⁺.

### Example 42. Synthesis of Compound 41

Step 1. To a solution of tert-butyl 2-bromoacetate (4.00 g, 20.5 mmol, 3.03 mL, 1.00 eq) in tetrahydrofuran (20 mL) was added zinc powder (13.4 g, 205 mmol, 10.0 eq) and chloro(trimethyl)silane (260 μL, 2.05 mmol, 0.10 eq). Then the mixture was stirred at 75° C. for 2 h. After reaction, the mixture was filtered to give crude product bromo-(2-tert-butoxy-2-oxo-ethyl)zinc (5.00 g, crude) as a yellow liquid.

Step 2. To a solution of bromo-(2-tert-butoxy-2-oxo-ethyl)zinc (2.00 g, 7.68 mmol, 1.00 eq) in tetrahydrofuran (10 mL) was added 4-bromo-2-chloro-1-methylbenzene (1.58 g, 7.68 mmol, 1.00 eq) and bis(tri-tert-butylphosphine)palladium(0) (392 mg, 0.768 mmol, 0.100 eq) under nitrogen atmosphere. Then the mixture was stirred at 75° C. for 2 h. The reaction mixture was concentrated to give a residue, which was purified by flash silica gel chromatography (ISCO®; 40 g SepaFlash® Silica Flash Column, Eluent of 0-30% ethyl acetate/petroleum ether gradient at 80 mL/min) to afford tert-butyl 2-(3-chloro-4-methylphenyl)acetate (1.20 g, 4.98 mmol, 65% yield) as a colorless oil. ¹H NMR (400 MHz, CDCl₃) δ=7.26 (d, J=1.5 Hz, 1H), 7.17 (d, J=7.8 Hz, 1H), 7.06 (dd, J=1.5, 7.8 Hz, 1H), 3.47 (s, 2H), 2.35 (s, 3H), 1.45 (s, 9H).

Step 3. A mixture of tert-butyl 2-(3-chloro-4-methylphenyl)acetate (500 mg, 2.08 mmol, 1.00 eq) in hydrochloric acid/dioxane (4 M, 20 mL) was stirred at 25° C. for 12 h. The reaction mixture was concentrated to afford 2-(3-chloro-4-methylphenyl)acetic acid (300 mg, crude) as a yellow oil. ¹H NMR (400 MHz, CDCl₃) δ=7.29 (d, J=1.2 Hz, 1H), 7.19 (d, J=7.7 Hz, 1H), 7.08 (dd, J=1.5, 7.7 Hz, 1H), 3.60 (s, 2H), 2.36 (s, 3H).

Step 4. To a solution of 2-(3-chloro-4-methylphenyl)acetic acid (65.0 mg, 0.352 mmol, 1.00 eq) and 3-(4-(3-aminoazetidin-1-yl)-2,6-difluorophenyl)piperidine-2,6-dione IX (104 mg, 0.352 mmol, 1.00 eq) in dimethyl formamide (2 mL) was added diisopropylethylamine (184 μL, 1.06 mmol, 3.00 eq) and O-(7-azabenzotriazol-1-yl)-N,N,N′,N′-tetramethyluronium hexafluorophosphate (174 mg, 0.458 mmol, 1.30 eq) at 25° C. Then the mixture was stirred at 25° C. for 1 h. The reaction was filtered. The filtrate was purified by Prep-HPLC (column: Phenomenex Luna C18 150*25 mm*10 μm; mobile phase: [water (formic acid)-acetonitrile]; B %: 34%-64%, 10 min) and Prep-HPLC (column: Waters Xbridge 150*25 mm*5 μm; mobile phase: [water (ammonium bicarbonate)-acetonitrile]; B %: 36%-66%, 8 min) to afford 2-(3-chloro-4-methylphenyl)-N-(1-(4-(2,6-dioxopiperidin-3-yl)-3,5-difluorophenyl)azetidin-3-yl)acetamide #41 (15.62 mg, 0.033 mmol, 9% yield, 99% purity) as a white solid. ¹H NMR (400 MHz, DMSO-d₆) δ=10.87 (br s, 1H), 8.77 (d, J=7.0 Hz, 1H), 7.30 (d, J=1.2 Hz, 1H), 7.27 (d, J=7.8 Hz, 1H), 7.11 (dd, J=1.4, 7.8 Hz, 1H), 6.17 (d, J=11.0 Hz, 2H), 4.58-4.47 (m, 1H), 4.10 (t, J=7.7 Hz, 2H), 4.03 (br dd, J=5.0, 12.6 Hz, 1H), 3.62 (dd, J=5.6, 7.8 Hz, 2H), 3.40 (br s, 2H), 2.85-2.70 (m, 1H), 2.48-2.44 (m, 1H), 2.28 (s, 3H), 2.07 (dq, J=3.5, 13.0 Hz, 1H), 1.99-1.87 (m, 1H). MS (ESI) m/z 462.2 [M+H]⁺.

### Example 43. Synthesis of Compound 42

Step 1. To a solution of 1-bromo-3,5-dimethylbenzene (1.32 mL, 9.73 mmol, 1.00 eq) and benzoyl peroxide (236 mg, 0.973 mmol, 0.100 eq) in acetonitrile (20 mL) was added N-bromosuccinimide (1.73 g, 9.73 mmol, 1.00 eq). The mixture was stirred at 90° C. for 2 h. The mixture was concentrated to give a residue. The residue was purified by silica gel chromatography (petroleum ether/ethyl acetate=100/1 to 10/1) to afford 1-bromo-3-(bromomethyl)-5-methylbenzene (1.70 g, 6.44 mmol, 66% yield) as a colorless oil. ¹H NMR (400 MHz, CDCl₃) δ=7.35 (s, 1H), 7.27 (s, 1H), 7.13 (s, 1H), 4.40 (s, 2H), 2.33 (s, 3H).

Step 2. To a solution of 1-bromo-3-(bromomethyl)-5-methylbenzene (1.70 g, 6.44 mmol, 1.00 eq) and tetrabutylammonium fluoride (1 M, 9.66 mL, 1.50 eq) in acetonitrile (10 mL) was dropwise added trimethylsilyl cyanide (1.21 mL, 9.66 mmol, 1.50 eq) at 0° C. The mixture was stirred at 25° C. for 2 h. The mixture was quenched by water (80 mL) and extracted with ethyl acetate (3×80 mL). The combined organic layers were washed with brine (80 mL), dried over anhydrous sodium sulfate, filtered and concentrated under reduced pressure to give a residue. The residue was purified by silica gel chromatography (petroleum ether/ethyl acetate=100/1 to 10/1) to afford 2-(3-bromo-5-methylphenyl) acetonitrile (900 mg, 4.28 mmol, 67% yield) as a colorless oil. ¹H NMR (400 MHz, CDCl₃) δ=7.31 (s, 1H), 7.28 (s, 1H), 7.09 (s, 1H), 3.70 (s, 2H), 2.35 (s, 3H).

Step 3. A mixture of 2-(3-bromo-5-methylphenyl)acetonitrile (300 mg, 1.43 mmol, 1.00 eq) in hydrochloric acid/methanol (4M, 3 mL) was stirred at 50° C. for 2 h. The reaction mixture was diluted with water (25 mL) and extracted with ethyl acetate (3×50 mL). The combined organic layers were washed with brine (20 mL), dried over anhydrous sodium sulfate, filtered and concentrated to afford methyl 2-(3-bromo-5-methylphenyl)acetate (340 mg, 1.40 mmol, 98% yield) as a colorless oil. ¹H NMR (400 MHz, CDCl₃) δ=7.25 (s, 2H), 7.02 (s, 1H), 3.71 (s, 3H), 3.56 (s, 2H), 2.32 (s, 3H).

Step 4. To a solution of methyl 2-(3-bromo-5-methylphenyl)acetate (200 mg, 0.823 mmol, 1.00 eq) and zinc cyanide (78.3 μL, 1.23 mmol, 1.50 eq) in dimethylformamide (2 mL) was added tetrakis(triphenylphosphine)palladium(0) (95.1 mg, 0.0823 mmol, 0.100 eq). The mixture was stirred at 100° C. for 12 h under nitrogen atmosphere. The mixture was quenched by water (30 mL), extracted with ethyl acetate (3×40 mL). The combined organic layers were washed with brine (40 mL), dried over anhydrous sodium sulfate, filtered and concentrated to give a residue. The residue was purified by Prep-TLC (petroleum ether/ethyl acetate=10/1) to afford methyl 2-(3-cyano-5-methylphenyl)acetate (20.0 mg, 0.106 mmol, 13% yield) as a colorless oil. ¹H NMR (400 MHz, CDCl₃) δ=7.39 (br s, 2H), 7.33 (s, 1H), 3.72 (s, 3H), 3.63 (s, 2H), 2.39 (s, 3H).

Step 5. To a solution of methyl 2-(3-cyano-5-methylphenyl)acetate (20.0 mg, 106 μmol, 1.00 eq) in methanol (1.20 mL) and water (0.400 mL) was added lithium hydroxide (5.06 mg, 211 μmol, 2.00 eq). The mixture was stirred at 25° C. for 2 h. The mixture was quenched by hydrochloride acid (1 M, 10 mL), extracted with ethyl acetate (3×20 mL). The combined organic layers were washed with brine (20 mL), dried over anhydrous sodium sulfate, filtered and concentrated to afford 2-(3-cyano-5-methylphenyl)acetic acid (30.0 mg, crude) as a yellow solid. ¹H NMR (400 MHz, CDCl₃) δ=7.41 (br s, 2H), 7.35 (s, 1H), 3.67 (s, 2H), 2.40 (s, 3H).

Step 6. To a solution of 3-(4-(3-aminoazetidin-1-yl)-2,6-difluorophenyl)piperidine-2,6-dione IX (55.0 mg, 161 μmol, 1.00 eq), 2-(3-cyano-5-methylphenyl)acetic acid (28.2 mg, 161 μmol, 1.00 eq), N,N-diisopropylethylamine (84.2 μL, 483 μmol, 3.00 eq) and 1-(3-dimethylaminopropyl)-3-ethylcarbodiimide hydrochloride (40.2 mg, 209 μmol, 1.30 eq) in dimethylformamide (1.00 mL) was added 1-hydroxybenzotriazole (21.8 mg, 161 μmol, 1.00 eq) at 0° C. The mixture was stirred at 25° C. for 1 h. The mixture was filtered. The filtrate was purified by Prep-HPLC (column: Phenomenex Synergi C18 150*25 mm*10 μm; mobile phase: [water (formic acid)-acetonitrile]; B %: 29%-59%, 10 min) and lyophilized to afford 2-(3-cyano-5-methylphenyl)-N-(1-(4-(2,6-dioxopiperidin-3-yl)-3,5-difluorophenyl)azetidin-3-yl)acetamide #42 (24.59 mg, 53.3 μmol, 33% yield, 98% purity) as a white solid. ¹H NMR (400 MHz, DMSO-d₆) δ=10.85 (br s, 1H), 8.80 (d, J=7.0 Hz, 1H), 7.53 (s, 1H), 7.50 (s, 1H), 7.41 (s, 1H), 6.17 (d, J=11.0 Hz, 2H), 4.59-4.50 (m, 1H), 4.10 (t, J=7.7 Hz, 2H), 4.03 (br dd, J=5.0, 12.6 Hz, 1H), 3.64 (dd, J=5.5, 7.8 Hz, 2H), 3.48 (s, 2H), 2.83-2.75 (m, 1H), 2.52 (br d, J=1.8 Hz, 1H), 2.33 (s, 3H), 2.14-2.03 (m, 1H), 1.97-1.90 (m, 1H). MS (ESI) m/z 453.2 [M+H]⁺.

### Example 44. Synthesis of Compound 43

Step 1. To a solution of 2-(5-chloro-2-fluoro-phenyl)acetic acid (10.0 g, 53.0 mmol, 1.00 eq) in methanol (100 mL) was added thionyl chloride (5.77 mL, 79.5 mmol, 1.50 eq). The mixture was stirred at 80° C. for 2 h. The mixture was concentrated under reduced pressure to remove methanol. The residue was diluted with water (200 mL) and extracted with ethyl acetate (3×100 mL). The combined organic phase was washed with brine (50 mL), dried over anhydrous sodium sulfate, filtered and concentrated under reduced pressure to afford methyl 2-(5-chloro-2-fluorophenyl)acetate (10.6 g, 52.0 mmol, 98% yield) as a yellow oil. ¹H NMR (400 MHz, DMSO-d₆) δ=7.52-7.42 (m, 1H), 7.41-7.34 (m, 1H), 7.28-7.19 (m, 1H), 3.76 (s, 2H), 3.64 (s, 3H)

Step 2. To a solution of methyl 2-(5-chloro-2-fluoro-phenyl)acetate (500 mg, 2.47 mmol, 1.00 eq) in tetrahydrofuran (5 mL) was added dropwise lithium bis(trimethylsilyl)amide (1.00 M, 7.40 mL, 3.00 eq) at 0° C. The mixture was stirred at 25° C. for 1 h. Then the mixture was added dropwise iodomethane (460 μL, 7.40 mmol, 3.00 eq) at 0° C. The mixture was stirred at 25° C. for 12 h. The residue was diluted with saturated ammonium chloride solution (60 mL) and extracted with ethyl acetate (3×30 mL). The combined organic layers were washed with brine (30 mL), dried over anhydrous sodium sulfate, filtered and concentrated under reduced pressure to give a residue. The residue was purified by column chromatography (SiO₂, petroleum ether/ethyl acetate=10/1 to 5/1) and concentrated in vacuum to afford methyl 2-(5-chloro-2-fluorophenyl)-2-methylpropanoate (300 mg, crude) as a yellow oil. ¹H NMR (400 MHz, DMSO-d6) δ=7.44 (dd, J=2.6, 6.9 Hz, 1H), 7.42-7.37 (m, 1H), 7.23 (dd, J=8.8, 11.0 Hz, 1H), 3.63-3.57 (m, 3H), 1.48 (s, 6H).

Step 3. To a solution of methyl 2-(5-chloro-2-fluoro-phenyl)-2-methyl-propanoate (300 mg, 1.30 mmol, 1.00 eq) in water (0.400 mL) and methanol (1.60 mL) was added lithium hydroxide (218 mg, 5.20 mmol, 4.00 eq) in portions at 25° C. Then the mixture was stirred at 50° C. for 12 h. The mixture was diluted with water (40 mL), extracted with ethyl acetate (3×30 mL). The pH of aqueous phase was adjusted to 5-6 by hydrochloric acid (1M, 10 mL) and extracted with ethyl acetate (3×30 mL), washed with brine (20 mL), dried over anhydrous sodium sulfate and filtered. The organic phase was concentrated to afford 2-(5-chloro-2-fluorophenyl)-2-methylpropanoic acid (145 mg, 0.629 mmol, 48% yield, 94% purity) as a white solid. ¹H NMR (400 MHz, DMSO-d₆) δ=12.47 (br s, 1H), 7.42-7.35 (m, 2H), 7.22 (dd, J=8.6, 10.9 Hz, 1H), 1.46 (s, 6H).

Step 4. To a solution of 3-(4-(3-aminoazetidin-1-yl)-2,6-difluorophenyl)piperidine-2,6-dione IX (110 mg, 372 μmol, 1.00 eq) in N,N-dimethyl formamide (2.00 mL) was added 2-(5-chloro-2-fluoro-phenyl)-2-methyl-propanoic acid (96.8 mg, 447 μmol, 1.20 eq), 0-(7-azabenzotriazol-1-yl)-N,N,N,N-tetramethyl uronium hexafluorophosphate (212 mg, 558 μmol, 1.50 eq), N,N-diisopropylethylamine (144 mg, 1.12 mmol, 194 μL, 3.00 eq) in portions and the mixture was stirred at 25° C. for 2 h. Then the mixture was heated to 50° C. and stirred for 12 h. The pH of the solution was adjusted to 5-6 by formic acid (0.1 mL) and filtered. The filtrate was purified by Prep-HPLC (column: Phenomenex Luna C18 150*25 mm*10 μm; mobile phase: [water (formic acid)-acetonitrile]; B %: 40%-70%, 10 min) and lyophilized to afford 2-(5-chloro-2-fluorophenyl)-N-(1-(4-(2,6-dioxopiperidin-3-yl)-3,5-difluorophenyl)azetidin-3-yl)-2-methylpropanamide #43 (35.37 mg, 68 μmol, 18% yield, 95% purity) as a white solid. ¹H NMR (400 MHz, DMSO-d₆) δ=10.85 (br s, 1H), 7.91 (br d, J=7.0 Hz, 1H), 7.45-7.31 (m, 2H), 7.24-7.13 (m, 1H), 6.13 (br d, J=11.1 Hz, 2H), 4.68-4.55 (m, 1H), 4.12-3.98 (m, 3H), 3.61 (br t, J=6.6 Hz, 2H), 2.82-2.71 (m, 1H), 2.57-2.56 (m, 1H), 2.12-2.01 (m, 1H), 1.99-1.86 (m, 1H), 1.43 (s, 6H). MS (ESI) m/z 494.2[M+H]⁺.

### Example 45. Synthesis of Compound 44

Step 1. To a solution of potassium tert-butoxide (10.2 g, 91.1 mmol, 4.00 eq) in tetrahydrofuran (50.0 mL) was added tosylmethyl isocyanide (8.90 g, 45.5 mmol, 2.00 eq) in tetrahydrofuran (20.0 mL) at −78° C. The mixture was stirred at −78° C. for 10 min. Then 3-bromo-5-chlorobenzaldehyde (5.00 g, 22.7 mmol, 1.00 eq) in tetrahydrofuran (50 mL) was added dropwise. The mixture was stirred at −78° C. for 1.5 h. Then methanol (10.0 mL) was added and the mixture was stirred at 20° C. for 1 h. The mixture was diluted with water (100 mL) and extracted with ethyl acetate (3×100 mL). The combined organic layers were dried over anhydrous sodium sulfate, filtered and concentrated under reduced pressure to give a residue. The residue was purified by column chromatography (SiO₂, petroleum ether/ethyl acetate=1/0 to 20/1) and concentrated under reduced pressure to afford 2-(3-bromo-5-chlorophenyl)acetonitrile (0.700 g, 3.04 mmol, 13% yield) as yellow oil. ¹H NMR (400 MHz, CDCl₃) δ=7.51 (t, J=1.8 Hz, 1H), 7.41 (td, J=0.8, 1.5 Hz, 1H), 7.32-7.29 (m, 1H), 3.73 (s, 2H).

Step 2. A mixture of 2-(3-bromo-5-chlorophenyl)acetonitrile (700 mg, 3.04 mmol, 1.00 eq) in hydrochloric acid (4 M in methanol, 2 mL) was stirred at 50° C. for 2 h. The mixture was concentrated under reduced pressure to give a residue. The residue was diluted with saturated aqueous sodium bicarbonate solution (20 mL) and extracted with ethyl acetate (3×20 mL). The combined organic layers were dried over anhydrous sodium sulfate, filtered and concentrated under reduced pressure to afford methyl 2-(3-bromo-5-chloro-phenyl)acetate (700 mg, 2.66 mmol, 87% yield) as a yellow oil. ¹H NMR (400 MHz, CDCl₃) δ=7.44 (t, J=1.8 Hz, 1H), 7.34 (t, J=1.5 Hz, 1H), 7.23 (t, J=1.6 Hz, 1H), 3.73 (s, 3H), 3.58 (s, 2H).

Step 3. To a solution of methyl 2-(3-bromo-5-chlorophenyl)acetate (650 mg, 2.47 mmol, 1.00 eq) in dimethylformamide (5 mL) were added zinc cyanide (156 μL, 2.47 mmol, 1.00 eq) and tetrakis[triphenylphosphine]palladium(0) (285 mg, 0.246 mmol, 0.100 eq). The mixture was stirred at 90° C. for 2 h under nitrogen atmosphere. The mixture was filtered. The filtrate was diluted with water (50 mL) and extracted with ethyl acetate (3×50 mL). The combined organic layers were washed with brine (50 mL), dried over anhydrous sodium sulfate, filtered and concentrated under reduced pressure to give a residue, which was purified by column chromatography (SiO₂, petroleum ether/ethyl acetate=1/0 to 30/1) and concentrated to afford methyl 2-(3-chloro-5-cyanophenyl)acetate (350 mg, 1.67 mmol, 67% yield) as colorless oil. ¹H NMR (400 MHz, CDCl₃) δ=7.56 (s, 1H), 7.54 (s, 1H), 7.49 (s, 1H), 3.74 (s, 3H), 3.66 (s, 2H).

Step 4. To a solution of methyl 2-(3-chloro-5-cyanophenyl)acetate (100 mg, 477 μmol, 1.00 eq) in methanol (1.50 mL) and water (0.500 mL) was added lithium hydroxide (60.0 mg, 1.43 mmol, 3.00 eq). The mixture was stirred at 20° C. for 2 h. The mixture was diluted with water (10 mL), extracted with ethyl acetate (10 mL). The pH of the aqueous phase was adjusted to 4-5 by adding hydrochloric acid solution and then extracted with ethyl acetate (2×10 mL). The combined organic phases were dried with anhydrous sodium sulfate, filtered and concentrated under reduced pressure to afford 2-(3-chloro-5-cyanophenyl)acetic acid (50.0 mg, crude) as a white solid.

Step 5. To a solution of 2-(3-chloro-5-cyanophenyl)acetic acid (40.0 mg, 204 μmol, 1.00 eq), 3-(4-(3-aminoazetidin-1-yl)-2,6-difluorophenyl)piperidine-2,6-dione IX (72.4 mg, 245 μmol, 1.20 eq), N,N-diisopropylethylamine (106 μL, 613 μmol, 3.00 eq), 1-(3-dimethylaminopropyl)-3-ethylcarbodiimide hydrochloride (50.9 mg, 265 μmol, 1.30 eq) in dimethylformamide (1.00 mL) was added 1-hydroxybenzotriazole (27.6 mg, 204 μmol, 1.00 eq) at 0° C. The mixture was stirred at 20° C. for 1 h. The pH of the mixture was adjusted to 5-6 with formic acid and filtered. The filtrate was purified by Prep-HPLC (column: Phenomenex Luna C18 150*25 mm*10 μm; mobile phase: [water (formic acid)-acetonitrile]; B %: 33%-63%, 9 min) and lyophilized to give crude product. It was triturated with tert-butyl methyl ether (5 mL) at 20° C. for 1 h and filtered. The filter cake was dried under reduced pressure to afford 2-(3-chloro-5-cyanophenyl)-N-(1-(4-(2,6-dioxopiperidin-3-yl)-3,5-difluorophenyl) azetidin-3-yl)acetamide #44 (12.38 mg, 29.3 μmol 14% yield, 99% purity) as a white solid. ¹H NMR (400 MHz, DMSO-d₆) δ=10.85 (s, 1H), 8.81 (d, J=6.9 Hz, 1H), 7.92 (t, J=1.7 Hz, 1H), 7.70 (d, J=1.5 Hz, 2H), 6.18 (d, J=11.0 Hz, 2H), 4.55 (br d, J=7.0 Hz, 1H), 4.11 (t, J=7.7 Hz, 2H), 4.03 (m, 1H), 3.65 (m, 2H), 3.56 (s, 2H), 2.84-2.73 (m, 1H), 2.52 (s, 1H), 2.14-2.02 (m, 1H), 1.98-1.90 (m, 1H). MS (ESI) m/z 473.1 [M+H]⁺.

### Example 46. Synthesis of Compound 45

Step 1. Sodium hydride (1.99 g, 49.7 mmol, 60% purity, 1.10 eq) was added into a solution of methanol (7.00 mL, 172 mmol, 3.82 eq) in N-methyl pyrrolidone (70.0 mL) at 0° C. The mixture was stirred at 0° C. for 1 h. Then 1,3-difluoro-5-nitrobenzene (7.20 g, 45.2 mmol, 1.00 eq) was added into this mixture at 0° C. The mixture was stirred at 25° C. for 2 h. Sodium hydride (905 mg, 22.6 mmol, 60% purity, 0.500 eq) was added into this mixture at 0° C. Then the mixture was stirred at 25° C. for 2 h. The mixture was quenched by 1M hydrochloric acid (100 mL) to adjust pH=3. The mixture was extracted with ethyl acetate (3×100 mL), washed with brine (300 mL), dried over anhydrous sodium sulfate, filtered and concentrated under reduced pressure to give a residue. The residue was purified by column chromatography (SiO₂, petroleum ether/ethyl acetate=100/0 to 100/1) to afford 1-fluoro-3-methoxy-5-nitrobenzene (5.30 g, 30.9 mmol, 68% yield) as a light green solid. ¹H NMR (400 MHz, CDCl₃) δ=7.58 (s, 1H), 7.55 (td, J=2.0, 8.2 Hz, 1H), 6.95 (td, J=2.3, 9.8 Hz, 1H), 3.91 (s, 3H).

Step 2. To a solution of 1-fluoro-3-methoxy-5-nitrobenzene (5.30 g, 30.9 mmol, 1.00 eq) in dichloromethane (55 mL) was added boron tribromide (8.95 mL, 92.9 mmol, 3.00 eq) at −78° C. The mixture was stirred at 25° C. for 2 h. The mixture was quenched with methanol (200 mL) and concentrated under reduced pressure to give a residue. The residue was purified by column chromatography (SiO₂, petroleum ether/ethyl acetate=1/0 to 10/1) to afford 3-fluoro-5-nitrophenol (4.00 g, 25.4 mmol, 82% yield) as an off-white solid. ¹H NMR (400 MHz, CDCl₃) δ=7.55 (s, 1H), 7.50 (td, J=2.0, 8.4 Hz, 1H), 7.08 (br s, 1H), 6.94 (td, J=2.2, 9.4 Hz, 1H).

Step 3. To a solution of 3-fluoro-5-nitrophenol (3.50 g, 22.2 mmol, 1.00 eq) in dimethylformamide (30 mL) and water (6 mL) were added into potassium carbonate (6.16 g, 44.5 mmol, 2.00 eq) and sodium 2-chloro-2,2-difluoroacetate (10.1 g, 66.8 mmol, 3.00 eq). The mixture was stirred at 100° C. for 2 h. The mixture was filtered and the filtrate was concentrated under reduced pressure to give a residue. The residue was dissolved with water (100 mL), extracted with ethyl acetate (3×50 mL). The combined organic phases were washed with brine (3×100 mL), dried over anhydrous sodium sulfate, filtered and concentrated under reduced pressure to give a residue. The residue was purified by column chromatography (SiO₂, petroleum ether/ethyl acetate=1/0 to 10/1) to afford 1-(difluoromethoxy)-3-fluoro-5-nitrobenzene (4.38 g, 21.1 mmol, 94% yield) as yellow oil. ¹H NMR (400 MHz, CDCl₃) δ=7.86-7.80 (m, 2H), 7.27-7.23 (m, 1H), 6.63 (t, J=71.6 Hz, 1H).

Step 4. To a solution of 1-(difluoromethoxy)-3-fluoro-5-nitrobenzene (4.50 g, 21.7 mmol, 1.00 eq) in methanol (40 mL) and water (20 mL) were added iron powder (3.64 g, 65.1 mmol, 3.00 eq) and ammonium chloride (5.81 g, 108 mmol, 5.00 eq). Then the mixture was stirred at 80° C. for 1 h. The reaction mixture was filtered and the filtrate was concentrated under reduced pressure to give a residue. The pH of the residue was adjusted to 8 by adding saturated sodium bicarbonate solution. The aqueous phase was extracted with ethyl acetate (3×100 mL). The combined organic phases were washed with brine (100 mL), dried over anhydrous sodium sulfate, filtered and concentrated under reduced pressure to afford 3-(difluoromethoxy)-5-fluoroaniline (3.60 g, 20.3 mmol, 93% yield) as yellow oil. ¹H NMR (400 MHz, DMSO-d₆) δ=7.13 (t, J=74.2 Hz, 1H), 6.21-6.14 (m, 2H), 6.11 (dd, J=2.1, 10.0 Hz, 1H), 5.72 (br s, 2H).

Step 5. To a solution of 2-(1-(tert-butoxycarbonyl)azetidin-3-yl)acetic acid (2.00 g, 9.29 mmol, 1.00 eq) in dichloromethane (20 mL) were added 2-(3H-[1,2,3]triazolo[4,5-b]pyridin-3-yl)-1,1,3,3-tetramethylisouronium hexafluorophosphate(V) (5.30 g, 13.9 mmol, 1.50 eq) and N,N-diisopropylethylamine (4.86 mL, 27.8 mmol, 3.00 eq). The mixture was stirred at 25° C. for 0.5 h. 3-(difluoromethoxy)-5-fluoroaniline (1.97 g, 11.1 mmol, 1.20 eq) was added into this mixture and was stirred at 25° C. for 11.5 h. The mixture was concentrated under reduced pressure to give a residue and was dissolved with water (50 mL), extracted with ethyl acetate (3×50 mL), washed with brine (100 mL), and dried over anhydrous sodium sulfate, filtered and concentrated under reduced pressure to give a residue. The residue was purified by column chromatography (SiO₂, petroleum ether/ethyl acetate=10/0 to 3/1) to give a crude product. The crude product was purified by reverse phase column chromatography (C18, 80 g; condition: water/acetonitrile=1/0 to 0/1, 0.1% formic acid) and lyophilized to afford tert-butyl 3-(2-((3-(difluoromethoxy)-5-fluorophenyl)amino)-2-oxoethyl)azetidine-1-carboxylate (2.20 g, 5.88 mmol, 73% yield) as a yellow oil. ¹H NMR (400 MHz, DMSO-d₆) δ=10.32 (s, 1H), 7.35 (td, J=1.9, 11.0 Hz, 1H), 7.25 (br s, 1H), 7.24 (s, J=73.6 Hz, 1H), 6.79 (td, J=2.1, 9.7 Hz, 1H), 4.00-3.91 (m, 2H), 3.57 (br s, 2H), 2.90-2.79 (m, 1H), 2.66 (d, J=7.8 Hz, 2H), 1.37 (s, 9H).

Step 6. To a solution of 3-(4-bromo-2,6-difluorophenyl)piperidine-2,6-dione (65.0 mg, 213 μmol, 1.00 eq) in dioxane (3 mL) were added 2-(azetidin-3-yl)-N-(3-(difluoromethoxy)-5-fluorophenyl)acetamide (222 mg, 320 μmol, 56% purity, 1.50 eq), cesium carbonate (208 mg, 641 μmol, 3.00 eq), 1,3-bis[2,6-bis(1-propylbutyl)phenyl]-4,5-dichloro-2H-imidazol-1-ium-2-ide;3-chloropyridine;dichloropalladium (20.7 mg, 21.3 μmol, 0.100 eq). Then the mixture was stirred at 100° C. for 12 h under nitrogen atmosphere. The mixture was filtered and the filtrate was concentrated under reduced pressure to give a residue. The residue was purified by Prep-TLC (SiO₂, petroleum ether: ethyl acetate=1:1) and concentrated under reduced pressure to give a crude product. The crude product was purified by Prep-HPLC (column: Waters xbridge 150*25 mm*10 μm; mobile phase: [water (ammonium bicarbonate)-acetonitrile]; B %: 33%-63%, 11 min) and lyophilized to afford N-(3-(difluoromethoxy)-5-fluorophenyl)-2-(1-(4-(2,6-dioxopiperidin-3-yl)-3,5-difluorophenyl)azetidin-3-yl)acetamide #45 (15.36 mg, 30.5 μmol, 14% yield, 99% purity) as a white solid. ¹H NMR (400 MHz, DMSO-d₆) δ=10.95-10.76 (m, 1H), 10.36 (s, 1H), 7.37 (td, J=1.9, 11.0 Hz, 1H), 7.27 (s, 1H), 7.24 (s, J=73.2 Hz, 1H), 6.80 (td, J=2.2, 9.7 Hz, 1H), 6.14 (s, 1H), 6.11 (s, 1H), 4.04 (br d, J=4.9 Hz, 1H), 4.01-3.97 (m, 2H), 3.56 (br t, J=6.3 Hz, 2H), 3.09-2.99 (m, 1H), 2.84-2.77 (m, 1H), 2.77-2.69 (m, 3H), 2.12-2.01 (m, 1H), 1.98-1.89 (m, 1H). MS (ESI) m/z 498.1 [M+H]⁺.

### Example 47. Synthesis of Compound 46

Step 1. To a solution of 5-chloro-2-fluorobenzoyl chloride (100 mg, 573 μmol, 1.00 eq) in dichloromethane (3 mL) was added N,N-dimethyl formamide (8.82 μL, 115 μmol, 0.200 eq), then oxalyl dichloride (100 μL, 1.15 mmol, 2.00 eq) was added into the mixture at 0° C. The mixture was stirred at 0° C. for 1 h. The reaction mixture was concentrated under reduced pressure to afford 5-chloro-2-fluorobenzoyl chloride (120 mg, crude) as a yellow solid.

Step 2. To a solution of 3-(4-(3-aminoazetidin-1-yl)-2,6-difluorophenyl)piperidine-2,6-dione IX (90.0 mg, 305 μmol, 1.00 eq) in N,N-dimethyl formamide (2 mL) was added triethylamine (127 μL, 914 μmol, 3.00 eq) and 5-chloro-2-fluorobenzoyl chloride (88.2 mg, 457 μmol, 1.50 eq) at 0° C. The mixture was stirred at 0° C. for 0.5 h. The mixture was filtered and the filtrate was purified by Prep-HPLC (column: Phenomenex C18 150 mm*25 mm*10 μm; mobile phase: [water (formic acid)-acetonitrile]; B %: 35%-65%, 8 min) and Prep-HPLC (column: Phenomenex Luna C18 150 mm*25 mm*10 μm; mobile phase: [water(formic acid)-acetonitrile]; B %: 34%-64%, 9 min). The desired fraction was collected and the aqueous solution was lyophilized to afford 5-chloro-N-(1-(4-(2,6-dioxopiperidin-3-yl)-3,5-difluorophenyl)azetidin-3-yl)-2-fluorobenzamide #46 (4.00 mg, 8.03 μmol, 36% yield) as a white solid. ¹H NMR (400 MHz, DMSO-d₆) δ=10.87 (br s, 1H), 9.15 (br d, J=6.3 Hz, 1H), 7.66 (br d, J=2.4 Hz, 1H), 7.63-7.52 (m, 1H), 7.37 (br t, J=9.3 Hz, 1H), 6.19 (br d, J=11.4 Hz, 2H), 4.77 (br d, J=6.1 Hz, 1H), 4.19 (br t, J=7.2 Hz, 2H), 4.04 (br d, J=8.5 Hz, 1H), 3.77 (br s, 2H), 2.83-2.72 (m, 1H), 2.64 (br dd, J=3.7, 5.3 Hz, 1H), 2.16-2.02 (m, 1H), 1.95 (br s, 1H). MS (ESI) m/z 452.0 [M+H]⁺.

### Example 48. Synthesis of Compound 47

Step 1. To a mixture of 3,4-difluorobenzonitrile (5.00 g, 35.9 mmol, 1.00 eq) in N,N-dimethylformamide (80 mL) was added dimethyl malonate (4.96 mL, 43.1 mmol, 1.20 eq) and potassium carbonate (14.9 g, 108 mmol, 3.00 eq) at 25° C. After stirring at 85-90° C. for 4 h, the mixture was partitioned between ethyl acetate (200 mL) and water (200 mL). The aqueous layer was extracted with ethyl acetate (2×200 mL). The combined organic layers were dried over sodium sulfate and evaporated to give a crude material. The crude material was purified by flash silica gel chromatography (petroleum ether/ethyl acetate=0/1 to 7/3) to afford dimethyl 2-(4-cyano-2-fluorophenyl)malonate (5.89 g, 22.2 mmol, 62% yield, 95% purity) as a white solid. MS (ESI) m/z 251.9 [M+H]⁺.

Step 2. To a mixture of dimethyl 2-(4-cyano-2-fluorophenyl)malonate (2.89 g, 11.5 mmol, 1.00 eq) in dimethyl sulfoxide (30 mL) was added lithium chloride (966 μL, 47.2 mmol, 4.10 eq) and water (1.00 mL, 55.5 mmol, 4.83 eq) at 25° C. After stirring at 130° C. for 5 h, the mixture was partitioned between ethyl acetate (60 mL) and water (60 mL). The aqueous layer was extracted with ethyl acetate (2×40 mL). The combined organic layers were dried over sodium sulfate and evaporated to give a crude material. The crude material was purified by flash silica gel chromatography (petroleum ether/ethyl acetate=0/1 to 7/3) to afford methyl 2-(4-cyano-2-fluorophenyl)acetate (1.00 g, 4.61 mmol, 40% yield, 89% purity) as a yellow solid. ¹H NMR (400 MHz, CDCl₃) δ=7.46-7.36 (m, 3H), 3.75 (s, 2H), 3.74 (s, 3H). MS (NEG) m/z 192.0 [M−H]⁺.

Step 3. To a solution of methyl 2-(4-cyano-2-fluorophenyl)acetate (200 mg, 1.04 mmol, 1.00 eq) in water (3 mL) and tetrahydrofuran (3 mL) was added lithium hydroxide (80.0 mg, 3.34 mmol, 3.23 eq). It was stirred at 25° C. for 2 h. The reaction mixture was diluted with water (30.0 mL) and exacted with ethyl acetate (3×30.0 mL). The organic phase was separated, washed with brine (2×10 mL), dried over anhydrous sodium sulfate, filtered and concentrated under reduced pressure to give a residue. The reaction mixture was concentrated under reduced pressure to give 2-(4-cyano-2-fluoro-phenyl)acetic acid (130 mg, crude) as a white solid. ¹H NMR (400 MHz, DMSO-d₆) δ=7.85-7.79 (m, 1H), 7.69-7.64 (m, 1H), 7.61-7.54 (m, 1H), 3.74 (s, 2H).

Step 4. To a solution of 3-(4-(3-aminoazetidin-1-yl)-2,6-difluorophenyl)piperidine-2,6-dione IX (80.0 mg, 270 μmol, 1.00 eq) and 2-(4-cyano-2-fluorophenyl)acetic acid (72.8 mg, 406 μmol, 1.50 eq) in dimethyl formamide (1 mL) was added diisopropylethylamine (61.3 μL, 352 μmol, 1.30 eq), 1H-benzo[ ][1,2,3]triazol-1-ol (36.6 mg, 270 μmol, 1.00 eq) and 1-(3-dimethylaminopropyl)-3-ethylcarbodiimide hydrochloride (51.9 mg, 270 μmol, 1.00 eq). The mixture was stirred at 25° C. for 2 h. The reaction mixture was diluted with water (30.0 mL) and exacted with ethyl acetate (3×30.0 mL). The organic phase was separated, washed with brine (2×10 mL), dried over anhydrous sodium sulfate, filtered and concentrated under reduced pressure to give a residue. The reaction mixture was concentrated under reduced pressure to give a residue. The crude product was dissolved in dimethyl formamide (2 mL) and purified by Prep-HPLC (column: Phenomenex Synergi C18 150*25 mm*10 μm; mobile phase: [water(formic acid)-acetonitrile]; B %: 26%-59%, 11 min) and lyophilized to give 2-(4-cyano-2-fluorophenyl)-N-(1-(4-(2,6-dioxopiperidin-3-yl)-3,5-difluorophenyl)azetidin-3-yl)acetamide #47 (25.0 mg, 51.4 μmol, 19% yield, 94% purity, formate) as a white solid. ¹H NMR (400 MHz, DMSO-d₆) δ=10.87 (br s, 1H), 8.90 (br d, J=6.60 Hz, 1H), 8.44 (br s, 1H), 7.80 (dd, J=9.72, 1.28 Hz, 1H), 7.66 (dd, J=7.83, 1.34 Hz, 1H), 7.59-7.50 (m, 1H), 6.18 (d, J=11.00 Hz, 2H), 4.60-4.0 (m, 1H), 4.11 (t, J=7.76 Hz, 2H), 4.03 (br dd, J=12.59, 5.14 Hz, 1H), 3.66-3.59 (m, 4H), 2.82-2.73 (m, 1H), 2.52-2.50 (m, 1H), 2.10-2.02 (m, 1H), 1.97-1.90 (m, 1H). MS (ESI) m/z 457.2 [M+H]⁺.

### Example 49. Synthesis of Compound 48

Step 1. To a solution of dimethyl malonate (4.43 mL, 38.6 mmol, 1.20 eq) in tetrahydrofuran (50 mL) was added sodium hydride (1.67 g, 41.8 mmol, 60% purity, 1.30 eq) in portions at 0° C. The mixture was stirred at 0° C. for 0.5 h. Then the mixture was added 2-chloro-4-fluorobenzonitrile (5.00 g, 32.1 mmol, 1.00 eq) in portions. The mixture was stirred at 80° C. for 12 h. The mixture was quenched with saturated ammonium chloride solution (30 mL) and extracted with ethyl acetate (3×10 mL). The combined organic layer was washed with brine (10 mL) and dried over sodium sulfate, filtered and concentrated to give crude product. The residue was purified by silica gel chromatography (SiO₂, petroleum ether/ethyl acetate=1/0 to 20/1) to give dimethyl 2-(3-chloro-4-cyanophenyl)malonate (1.50 g, 5.60 mmol, 17% yield) as a white solid.

¹H NMR (400 MHz, CDCl₃) δ=7.70 (d, J=8.0 Hz, 1H), 7.63 (d, J=1.6 Hz, 1H), 7.46 (dd, J=1.6, 8.0 Hz, 1H), 4.68 (s, 1H), 3.81 (s, 6H).

Step 2. To a solution of dimethyl 2-(3-chloro-4-cyano-phenyl)propanedioate (500 mg, 1.87 mmol, 1.00 eq) in methanol (1.20 mL) and water (0.400 mL) was added lithium hydroxide (224 mg, 9.34 mmol, 5.00 eq). The mixture was stirred at 25° C. for 12 h. The mixture was adjusted to pH=7 with 1N hydrochloric acid aqueous solution. The reaction mixture was diluted with water (30 mL), extracted with ethyl acetate (3×10 mL). The combined organic layer was washed with brine (30 mL), dried over anhydrous sodium sulfate, filtered and concentrated under reduced pressure to afford 2-(3-chloro-4-cyano-phenyl)acetic acid (200 mg, 1.02 mmol, 55% yield) as a white solid. ¹H NMR (400 MHz, DMSO-d₆) δ=7.95-7.78 (m, 1H), 7.63 (s, 1H), 7.41 (d, J=7.9 Hz, 1H), 3.62 (s, 2H).

Step 3. To a solution of 3-(4-(3-aminoazetidin-1-yl)-2,6-difluorophenyl)piperidine-2,6-dione (100 mg, 0.339 mmol, 1.00 eq) and 2-(3-chloro-4-cyanophenyl)acetic acid (79.5 mg, 0.406 mmol, 1.20 eq), diisopropylethylamine (177 μL 1.02 mmol, 3.00 eq), 1-(3-dimethylaminopropyl)-3-ethylcarbodiimide hydrochloride (97.4 mg, 0.508 mmol, 1.50 eq) in dimethyl formamide (2 mL) was added 1-hydroxybenzotriazole (68.6 mg, 0.508 mmol, 1.50 eq) at 0° C. The reaction mixture was stirred at 25° C. for 1 h. The mixture was filtered and the filtrate was purified by reversed phase column chromatography (C18, 80 g; condition: water/acetonitrile=1/0 to 0/1, 0.1% formic acid) and lyophilized to afford 2-(3-chloro-4-cyanophenyl)-N-(1-(4-(2,6-dioxopiperidin-3-yl)-3,5-difluorophenyl)azetidin-3-yl)acetamide #48 (7.00 mg, 0.0147 mmol, 4% yield, 99% purity) as a white solid. ¹H NMR (400 MHz, DMSO-d₆) δ=10.86 (s, 1H), 8.85 (br d, J=6.2 Hz, 1H), 7.92 (d, J=8.1 Hz, 1H), 7.65 (s, 1H), 7.43 (d, J=7.6 Hz, 1H), 6.18 (br d, J=11.1 Hz, 2H), 4.59-4.50 (m, 1H), 4.11 (br t, J=7.8 Hz, 2H), 4.07-4.00 (m, 1H), 3.61-3.67 (m, 2H), 3.59 (s, 2H), 2.84-2.72 (m, 1H), 2.60-2.56 (m, 1H), 2.14-2.01 (m, 1H), 1.99-1.90 (m, 1H). MS (ESI) m/z. 473.1 [M+H]⁺.

### Example 50. Synthesis of Compound 49

Step 1. To a solution of 2,4,6-trimethylpyridine (1.09 mL, 8.25 mmol, 1.00 eq) in tetrahydrofuran (30 mL) was added lithium diisopropylamide (2 M, 8.25 mL, 2.00 eq) at −10° C. under nitrogen atmosphere. The mixture was stirred at −10° C. for 0.5 h. Dimethyl carbonate (695 μL, 8.25 mmol, 1.00 eq) was added into the mixture and the mixture was stirred at 25° C. for 1.5 h. The mixture was quenched by saturated ammonium chloride solution (100 mL) at 0° C. The mixture was extracted with ethyl acetate (3×80 mL). The organic layers were washed with brine (60 mL), dried over anhydrous sodium sulfate, filtered and concentrated to afford a residue. The residue was purified by silica gel chromatography (petroleum ether/ethyl acetate=10/1 to 6/1) to afford methyl 2-(2,6-dimethylpyridin-4-yl)acetate (100 mg, 0.558 mmol, 7% yield) as a yellow oil. ¹H NMR (400 MHz, DMSO-d₆) δ=6.93 (s, 2H), 3.64 (s, 2H), 3.62 (s, 3H), 2.39 (s, 6H).

Step 2. To a solution of methyl 2-(2,6-dimethylpyridin-4-yl)acetate (100 mg, 0.558 mmol, 1.00 eq) in methanol (1.20 mL) and water (0.400 mL) was added lithium hydroxide monohydrate (46.8 mg, 1.12 mmol, 2.00 eq). The mixture was stirred at 25° C. for 2 h. The mixture was added hydrochloric acid (1 M, 2.5 mL) and lyophilized to afford 2-(2,6-dimethylpyridin-4-yl)acetic acid (90.0 mg, 0.545 mmol, 98% yield) as a yellow solid. ¹H NMR (400 MHz, DMSO-d₆) δ=13.56-12.54 (m, 1H), 7.60 (s, 2H), 3.88 (s, 2H), 2.71 (s, 6H).

Step 3. To a solution of 3-(4-(3-aminoazetidin-1-yl)-2,6-difluorophenyl)piperidine-2,6-dione IX (80.0 mg, 234 μmol, 1.00 eq, formate), 2-(2,6-dimethylpyridin-4-yl)acetic acid (38.7 mg, 234 μmol, 1.00 eq), N,N-diisopropylethylamine (122 μL, 703 μmol, 3.00 eq) and 1-(3-dimethylaminopropyl)-3-ethylcarbodiimide hydrochlorid (58.4 mg, 305 μmol, 1.30 eq) in dimethylformamide (1 mL) was added 1-hydroxybenzotriazole (31.7 mg, 234 μmol, 1.00 eq) at 0° C. and was stirred at 25° C. for 1 h. The mixture was filtered. The filtrate was purified by Prep-HPLC (column: Phenomenex Synergi C18 150*25 mm*10 μm; mobile phase: [water (formic acid)-acetonitrile]; B %: 1%-31%, 10 min) and further purified by Prep-HPLC (column: Phenomenex Synergi C18 150*25 mm*10 μm; mobile phase: [water (formic acid)-acetonitrile]; B %: 1%-31%, 10 min) and lyophilized to give 2-(2,6-dimethylpyridin-4-yl)-N-(1-(4-(2,6-dioxopiperidin-3-yl)-3,5-difluorophenyl)azetidin-3-yl)acetamide #49 (22.91 mg, 46 μmol, 20% yield, 99% purity, formate) as an off-white solid. ¹H NMR (400 MHz, DMSO-d₆) δ=10.86 (s, 1H), 8.81 (br d, J=6.8 Hz, 1H), 6.91 (s, 2H), 6.18 (d, J=11.1 Hz, 2H), 4.59-4.50 (m, 1H), 4.11 (t, J=7.7 Hz, 2H), 4.04 (br dd, J=5.0, 12.5 Hz, 1H), 3.65-3.61 (m, 2H), 3.38 (s, 2H), 2.83-2.73 (m, 1H), 2.53-2.51 (m, 1H), 2.38 (s, 6H), 2.15-2.03 (m, 1H), 1.98-1.90 (m, 1H). MS (ESI) m/z 443.2 [M+H]⁺.

### Example 51. Synthesis of Compound 50

Step 1. To a solution of (3,5-dimethylphenyl)boronic acid (0.300 g, 2.00 mmol, 1.00 eq), ethyl 2-bromo-2,2-difluoroacetate (514 μL, 4.00 mmol, 2.00 eq), 4,5-bis(diphenylphosphino)-9,9-dimethylxanthene (116 mg, 0.200 mmol, 0.100 eq), cuprous iodide (19.1 mg, 0.100 mmol, 0.0500 eq) and potassium carbonate (1.11 g, 8.00 mmol, 4.00 eq) in dioxane (5 mL) was added tetrakis(triphenylphosphine)palladium(0) (116 mg, 0.100 mmol, 0.0500 eq). The mixture was stirred at 80° C. for 12 h. The mixture was added water (40 mL), and then extracted with ethyl acetate (3×40 mL). The combined organic layers were washed with brine (3×50 mL), dried over anhydrous sodium sulfate, filtered and concentrated under reduced pressure to give a residue. The residue was purified by silica gel chromatography (petroleum ether/ethyl acetate=100/1-10/1) to afford ethyl 2-(3,5-dimethylphenyl)-2,2-difluoroacetate (110 mg, 0.482 mmol, 24% yield) as colorless oil. ¹H NMR (400 MHz, DMSO-d₆) δ=7.22 (s, 1H), 7.18 (s, 2H), 4.30 (q, J=7.2 Hz, 2H), 2.33 (s, 6H), 1.23 (t, J=7.1 Hz, 3H).

Step 2. To a solution of ethyl 2-(3,5-dimethylphenyl)-2,2-difluoroacetate (70.0 mg, 307 μmol, 1.00 eq) in methanol (1.20 mL) and water (0.500 mL) was added lithium hydroxide monohydrate (25.7 mg, 613 μmol, 2.00 eq). The mixture was stirred at 25° C. for 2 h. The mixture was added hydrochloric acid (1 M, 3 mL) to adjust pH=4, extracted with ethyl acetate (3×20 mL). The combined organic layers were washed with brine (10 mL), dried over anhydrous sodium sulfate, filtered and concentrated under reduced pressure to give 2-(3,5-dimethylphenyl)-2,2-difluoroacetic acid (60.0 mg, 300 μmol, 98% yield) as yellow oil. ¹H NMR (400 MHz, DMSO-d₆) δ=7.19 (s, 1H), 7.16 (s, 2H), 2.32 (s, 6H).

Step 3. To a solution of 3-(4-(3-aminoazetidin-1-yl)-2,6-difluorophenyl)piperidine-2,6-dione IX (70.0 mg, 205 μmol, 1.00 eq, formate), 2-(3,5-dimethylphenyl)-2,2-difluoroacetic acid (41.1 mg, 205 μmol, 1.00 eq), N,N-diisopropylethylamine (107 μL 615 μmol, 3.00 eq) and 1-(3-dimethylaminopropyl)-3-ethylcarbodiimide hydrochlorid (51.1 mg, 267 μmol, 1.30 eq) in dimethylformamide (1 mL) was added 1-hydroxybenzotriazole (27.7 mg, 205 μmol, 1.00 eq) at 0° C. The mixture was stirred at 25° C. for 1 h. The mixture was filtered and the filtrate was purified by Prep-HPLC (column: Phenomenex Synergi C18 150*25 mm*10 μm; mobile phase: [water (formic acid)-acetonitrile]; B %: 48%-68%, 10 min) and lyophilized to afford 2-(3,5-dimethylphenyl)-N-(1-(4-(2,6-dioxopiperidin-3-yl)-3,5-difluorophenyl)azetidin-3-yl)-2,2-difluoroacetamide #50 (16.64 mg, 34.5 μmol, 17% yield, 99% purity) as a white solid. ¹H NMR (400 MHz, DMSO-d₆) δ=10.86 (br s, 1H), 9.53 (br d, J=7.0 Hz, 1H), 7.18 (s, 3H), 6.18 (d, J=11.0 Hz, 2H), 4.69-4.60 (m, 1H), 4.13 (t, J=7.6 Hz, 2H), 4.04 (br dd, J=5.1, 12.5 Hz, 1H), 3.80-3.72 (m, 2H), 2.89-2.73 (m, 1H), 2.55-2.52 (m, 1H), 2.32 (s, 6H), 2.13-2.02 (m, 1H), 1.97-1.89 (m, 1H). MS (ESI) m/z 478.2 [M+H]⁺.

### Example 52. Synthesis of Compound 51

Step 1. To a solution of 2,4,6-trimethylpyridine (1.09 mL, 8.25 mmol, 1.00 eq) in tetrahydrofuran (30 mL) was added lithium diisopropylamide (2 M, 8.25 mL, 2.00 eq) at −10° C. under nitrogen atmosphere. The mixture was stirred at −10° C. for 0.5 h. Dimethyl carbonate (695 μL, 8.25 mmol, 1.00 eq) was added to the mixture and the mixture was stirred at 20° C. for 1.5 h. The mixture was quenched by saturated ammonium chloride solution (100 mL) at 0° C. The mixture was extracted with ethyl acetate (3×80 mL). The organic layers were washed with brine (60 mL), dried over anhydrous sodium sulfate, filtered and concentrated to afford a residue. The residue was purified by silica gel chromatography (petroleum ether/ethyl acetate=10/1-6/1) to afford methyl 2-(4,6-dimethylpyridin-2-yl)acetate (170 mg, 0.949 mmol, 11% yield) as yellow oil. ¹H NMR (400 MHz, DMSO-d₆) δ=6.96 (s, 2H), 3.73 (s, 2H), 3.61 (s, 3H), 2.37 (s, 3H), 2.25 (s, 3H).

Step 2. To a solution of methyl 2-(4,6-dimethylpyridin-2-yl)acetate (100 mg, 0.558 mmol, 1.00 eq) in methanol (1.20 mL) and water (0.400 mL) was added lithium hydroxide monohydrate (46.8 mg, 1.12 mmol, 2.00 eq). The mixture was stirred at 25° C. for 2 h. The mixture was added to hydrochloric acid (1M, 2.5 mL) and lyophilized to give 2-(4,6-dimethylpyridin-2-yl)acetic acid (80.0 mg, 0.484 mmol, 88% yield) as a white solid. ¹H NMR (400 MHz, DMSO-d₆) δ=7.62 (br d, J=2.1 Hz, 2H), 4.17 (s, 2H), 2.69 (s, 3H), 2.51-2.50 (m, 3H).

Step 3. To a solution of 3-(4-(3-aminoazetidin-1-yl)-2,6-difluorophenyl)piperidine-2,6-dione IX (80.0 mg, 234 μmol, 1.00 eq, formate), 2-(4,6-dimethylpyridin-2-yl)acetic acid (38.7 mg, 234 μmol, 1.00 eq), N,N-diisopropylethylamine (122 μL, 703 μmol, 3.00 eq) and 1-(3-dimethylaminopropyl)-3-ethylcarbodiimide hydrochlorid (58.4 mg, 305 μmol, 1.30 eq) in dimethylformamide (1 mL) was added 1-hydroxybenzotriazole (31.7 mg, 234 μmol, 1.00 eq) at 0° C. The mixture was stirred at 25° C. for 1 h. The mixture was filtered. The filtrate was purified by Prep-HPLC (column: Phenomenex Synergi C18 150*25 mm*10 μm; mobile phase: [water (formic acid)-acetonitrile]; B %: 3%-33%, 10 min) and lyophilized to afford 2-(4,6-dimethylpyridin-2-yl)-N-(1-(4-(2,6-dioxopiperidin-3-yl)-3,5-difluorophenyl)azetidin-3-yl) acetamide #51 (54.02 mg, 121 μmol, 52% yield, 99% purity) as an off-white solid. ¹H NMR (400 MHz, DMSO-d₆) δ=10.86 (s, 1H), 8.78 (d, J=6.8 Hz, 1H), 6.94 (d, J=3.5 Hz, 2H), 6.17 (d, J=11.1 Hz, 2H), 4.61-4.51 (m, 1H), 4.11 (t, J=7.6 Hz, 2H), 4.04 (br dd, J=5.1, 12.7 Hz, 1H), 3.68-3.62 (m, 2H), 3.52 (s, 2H), 2.84-2.72 (m, 1H), 2.53-2.52 (m, 1H), 2.37 (s, 3H), 2.24 (s, 3H), 2.13-2.02 (m, 1H), 1.99-1.91 (m, 1H). MS (ESI) m/z 443.2 [M+H]⁺.

### Example 53. Synthesis of Compound 52

Step 1. To a solution of 2-(2-bromo-4-chlorophenyl)acetic acid (300 mg, 1.20 mmol, 1.00 eq), methylboronic acid (216 mg, 3.61 mmol, 3.00 eq) and potassium carbonate (499 mg, 3.61 mmol, 3.00 eq) in dioxane (10 mL) was added tetrakis(triphenylphosphine)palladium(0) (139 mg, 120 μmol, 0.100 eq). The mixture was stirred at 100° C. for 12 h under nitrogen atmosphere. The mixture was quenched with water (40 mL), and then extracted with ethyl acetate (3×40 mL). The combined organic layers were washed with brine (3×50 mL), dried over anhydrous sodium sulfate, filtered and concentrated under reduced pressure to give a residue, which was purified by reversed phase (C18, 120 g; condition: water/acetonitrile=1/0 to 0/1, 0.1% formic acid) and lyophilized to give 2-(4-chloro-2-methylphenyl)acetic acid (120 mg, 650 μmol, 54% yield) as a yellow solid. ¹H NMR (400 MHz, DMSO-d₆) δ=12.77-11.90 (m, 1H), 7.26 (s, 1H), 7.21-7.17 (m, 2H), 3.58 (s, 2H), 2.22 (s, 3H).

Step 2. To a solution of 3-(4-(3-aminoazetidin-1-yl)-2,6-difluorophenyl)piperidine-2,6-dione IX (80.0 mg, 234 μmol, 1.00 eq, formate), 2-(4-chloro-2-methylphenyl)acetic acid (43.3 mg, 234 μmol, 1.00 eq) in dimethylformamide (1 mL) was added N,N-diisopropylethylamine (122 μL, 703 μmol, 3.00 eq), 1-hydroxybenzotriazole (31.7 mg, 234 μmol, 1.00 eq) and 1-(3-dimethylaminopropyl)-3-ethylcarbodiimide hydrochlorid (58.4 mg, 305 μmol, 1.30 eq) at 0° C. The mixture was stirred at 25° C. for 1 h. The mixture was filtered. The filtrate was purified by Prep-HPLC (column: Phenomenex Synergi C18 150*25 mm*10 μm; mobile phase: [water (formic acid)-acetonitrile]; B %: 42%-62%, 10 min) and lyophilized to give 2-(4-chloro-2-methylphenyl)-N-(1-(4-(2,6-dioxopiperidin-3-yl)-3,5-difluorophenyl)azetidin-3-yl)acetamide #52 (55.41 mg, 116 μmol, 49% yield, 97% purity) as a white solid. ¹H NMR (400 MHz, DMSO-d₆) δ=10.86 (br s, 1H), 8.72 (d, J=7.0 Hz, 1H), 7.23 (s, 1H), 7.21-7.15 (m, 2H), 6.17 (d, J=11.0 Hz, 2H), 4.61-4.52 (m, 1H), 4.11 (t, J=7.6 Hz, 2H), 4.03 (br dd, J=5.3, 12.6 Hz, 1H), 3.64 (dd, J=5.6, 7.7 Hz, 2H), 3.46 (s, 2H), 2.83-2.73 (m, 1H), 2.55-2.52 (m, 1H), 2.24 (s, 3H), 2.13-2.03 (m, 1H), 1.98-1.89 (m, 1H). MS (ESI) m/z 462.1 [M+H]⁺.

### Example 54. Synthesis of Compound 53

To a solution of 3-(4-(3-aminoazetidin-1-yl)-2,6-difluorophenyl)piperidine-2,6-dione IX (70.0 mg, 237 μmol, 1.00 eq) and 2-(4-chloro-3-methylphenyl)acetic acid (65.7 mg, 356 μmol, 1.50 eq) in dimethylformamide (1 mL) was added N,N-diisopropylethylamine (124 μL, 711 μmol, 3.00 eq). The reaction mixture was stirred at 20° C. at 0.5 h. Then the 1,2-diphenylhydrazine (32.0 mg, 237 μmol, 1.00 eq) and 1-(3-dimethylaminopropyl)-3-ethylcarbodiimide hydrochloride (59.1 mg, 308 μmol, 1.30 eq) were added into the mixture at 0° C. and the mixture was stirred at 20° C. for 1.5 h. The mixture was filtered. The filtrate was purified by Prep-HPLC (column: Phenomenex luna C18 150*25 mm*10 μm; mobile phase: [water (0.225% formic acid)-acetonitrile]; B %: 36%-66%, 10 min) and lyophilized to afford 2-(4-chloro-3-methylphenyl)-N-(1-(4-(2,6-dioxopiperidin-3-yl)-3,5-difluorophenyl)azetidin-3-yl)acetamide #53 (27.97 mg, 60.0 μmol, 25% yield, 99% purity) as a white solid. ¹H NMR (400 MHz, DMSO-d₆) δ=10.86 (s, 1H), 8.75 (d, J=6.90 Hz, 1H), 7.33 (d, J=8.16 Hz, 1H), 7.22 (s, 1H), 7.10 (dd, J=8.34, 1.57 Hz, 1H), 6.18 (s, 1H), 6.16 (s, 1H), 4.53 (m, 1H), 4.10 (t, J=7.78 Hz, 2H), 4.03 (br dd, J=12.74, 4.71 Hz, 1H), 3.62 (dd, J=7.53, 5.52 Hz, 2H), 3.39 (s, 2H), 2.77 (m, 1H), 2.53 (m, 1H), 2.30 (s, 3H), 2.07 (m, 1H), 1.94 (m, 1H). MS (ESI) m/z 462.1 [M+H]⁺.

### Example 55. Synthesis of Compound 54

Step 1. To a solution of 2-(1-(tert-butoxycarbonyl)azetidin-3-yl)acetic acid (5.00 g, 23.2 mmol, 1.00 eq) and O-(7-azabenzotriazol-1-yl)-N,N,N,N-tetramethyluroniumhexafluorophosphate (13.3 g, 34.8 mmol, 1.50 eq) in dichloromethane (50 mL) was added N,N-diisopropylethylamine (12.1 mL, 69.7 mmol, 3.00 eq). The mixture was stirred at 25° C. for 0.5 h. Then 3,5-dimethylaniline (3.48 mL, 27.9 mmol, 1.20 eq) was added into the mixture and stirred at 25° C. for 1.5 h. The mixture was concentrated to afford a residue. The residue was purified by silica gel chromatography (petroleum ether/ethyl acetate=10/1 to 3/1) to afford tert-butyl 3-(2-((3,5-dimethylphenyl)amino)-2-oxoethyl)azetidine-1-carboxylate (7.00 g, 22.0 mmol, 94% yield) as a yellow solid. ¹H NMR (400 MHz, DMSO-d₆) δ=9.78 (s, 1H), 7.19 (s, 2H), 6.67 (s, 1H), 3.94 (br s, 2H), 3.56 (br s, 2H), 2.87-2.79 (m, 1H), 2.60 (d, J=7.8 Hz, 2H), 2.21 (s, 6H), 1.37 (s, 9H).

Step 2. To a solution of tert-butyl 3-(2-((3,5-dimethylphenyl) amino)-2-oxoethyl)azetidine-1-carboxylate (400 mg, 1.26 mmol, 1.00 eq) in dichloromethane (10 mL) was added trifluoroacetic acid (2.00 mL, 27.0 mmol, 21.5 eq). The mixture was stirred at 25° C. for 1 h. The mixture was concentrated to remove dichloromethane and added saturated aqueous sodium carbonate solution (8 mL) to pH=7 and filtered. The filtrate was purified by reversed phase (C18, 120 g; condition: water/acetonitrile=1/0 to 0/1, 0.1% formic acid) and lyophilized to afford 2-(azetidin-3-yl)-N-(3,5-dimethylphenyl) acetamide (280 mg, 1.06 mmol, 84% yield) as yellow oil. ¹H NMR (400 MHz, CDCl₃) δ=9.68-9.20 (m, 1H), 8.09 (br s, 1H), 7.13 (s, 2H), 6.76 (s, 1H), 4.29 (br t, J=9.4 Hz, 2H), 3.89 (br dd, J=6.4, 10.1 Hz, 2H), 3.37-3.27 (m, 1H), 2.87 (br d, J=7.8 Hz, 2H), 2.30 (br s, 1H), 2.28 (s, 6H).

Step 3. To a solution of 3-(4-bromo-2,6-difluorophenyl)piperidine-2,6-dione (320 mg, 1.05 mmol, 1.00 eq), 2-(azetidin-3-yl)-N-(3,5-dimethylphenyl)acetamide (278 mg, 1.05 mmol, 1.00 eq), palladium(II) acetate (23.6 mg, 0.105 mmol, 0.100 eq) and 4,5-bis(diphenylphosphino)-9,9-dimethylxanthene (60.9 mg, 0.105 mmol, 0.100 eq) in dioxane (6 mL) was added cesium carbonate (1.03 g, 3.16 mmol, 3.00 eq). The mixture was stirred at 100° C. for 12 h. The mixture was filtered and concentrated to afford a residue. The residue was purified by Prep-NPLC (column: Welch Ultimate XB-SiOH 250*50*10 μm; mobile phase: [hexane-ethyl alcohol]; B %: 1%-40%, 15 min) and further purified by Prep-HPLC (column: Waters Xbridge 150*25 mm*5 μm; mobile phase: [water (ammonium bicarbonate)-acetonitrile]; B %: 28%-58%, 10 min) and lyophilized to afford N-(3,5-dimethylphenyl)-2-(1-(4-(2,6-dioxopiperidin-3-yl)-3,5-difluorophenyl) azetidin-3-yl)acetamide #54 (31.18 mg, 0.699 mmol, 7% yield, 99% purity) as a yellow solid. ¹H NMR (400 MHz, DMSO-d₆) δ=10.85 (br s, 1H), 9.81 (s, 1H), 7.20 (s, 2H), 6.67 (s, 1H), 6.12 (d, J=11.1 Hz, 2H), 4.04 (br d, J=5.4 Hz, 1H), 3.98 (t, J=7.5 Hz, 2H), 3.56 (br t, J=6.3 Hz, 2H), 3.07-2.98 (m, 1H), 2.83-2.73 (m, 1H), 2.68 (d, J=7.6 Hz, 2H), 2.52 (br d, J=2.0 Hz, 1H), 2.22 (s, 6H), 2.12-2.03 (m, 1H), 1.98-1.90 (m, 1H). MS (ESI) m/z 442.3 [M+H]⁺.

### Example 56: Compound Binding to CRBN by HTRF Assay

Compound activity was monitored in a Homogenous Time-Resolved Fluorescence (HTRF) assay using 1-[5-({2-[2-(2-{[2-(2,6-dioxopiperidin-3-yl)-1,3-dioxo-2,3-dihydro-1H-isoindol-4-yl]oxy}acetamido)ethoxy]ethyl}carbamoyl)pentyl]-3,3-dimethyl-2-[(1E,3E)-5-[(2E)-1,3,3-trimethyl-5-sulfo-2,3-dihydro-1H-indol-2-ylidene]penta-1,3-dien-1-yl]-3H-indol-1-ium-5-sulfonate as a fluorescent probe. Biochemical assays were conducted in Greiner white 384 well HiBase plates (Cat. No 784075-25) in 10 μL total volume. A one pot detection solution of CRBN-DDB1 (2.5 nM), Anti-His Terbium Cryptate Gold (1×, PerkinElmer Cat. #: 61HI2TLB), and Cy5-Thalidomide (100 nM, Tenova Cat.: T52461) was prepared in 20 mM HEPES, 20 mM NaCl, 0.2 mM TCEP, 0.2 mM EDTA, and 0.005% Tween20 was dispensed to each assay plate. Compounds were stored in dry, ambient temperatures at 10 mM. A 10-point, 1:3 dilution series was prepared from 10 mM stock concentrations in Echo-compatible LDV plates. 10 nL of each compound dilution series was dispensed into assays wells using an Echo 650 (Labcyte inc. USA). 10 nL of 10 mM Lenalidomide was transferred into the active-control wells for the assay and 10 nL of DMSO was transferred into the neutral-control wells. The assay was then allowed to incubate for 30 min at ambient temperature after transferring compound. Plate measurements were taken on a Pherastar FSX (BMG Labtech, Germany) using the HTRF Red filter (Ex. 337 nm, em1: 620 nm, em2: 665 nm) (Flashes: 50, Integration time: 60-400 us, Z-height: 10 mm, Ratio-multiplier: 10,000). The HTRF signal was then subsequently normalized to the neutral and active controls. Analysis and IC₅₀ values were derived using KNIME analytics (KNIME Zurich) transformation and fitting within Collaborative Drug Discovery (Collaborative Drug Discovery USA). Kᵢ was derived from the geometric mean of the IC50 values using the Cheng-Prustoff transformation.

### Example 57: Compound Activity by Immunofluorescence Assay

The representative compounds were tested in an immunofluorescence assay for their activity to degrade GSPT1. CAL-51 cells were purchased from DSMZ (cat. Number ACC302), sub-cultured in 90% Dulbecco's MEM (4.5 g/L glucose, Gibco 11965)+10% heat inactivated FBS (BioConcept, 2-01F136I) and incubated at 37° C., 5% CO₂. For the assay imaging microtiter plate Cell Carrier 96 Ultra (Perkin Elmer 6055302) were pre-coated with Fibronectin (Sigma F085, 30 μl at 0.2 μg/mL) in PBS (100 μL, Gibco 14190) for 45 min at room temperature, rinsed with PBS and CAL-51 cells (30K cells/well) were plated and let to adhere overnight. Cells were treated with compounds typically using a serial dilution ranging from 30 μM to 0.1 nM for 6 h. Compounds were stored at 10 mM DMSO stocks. Vehicle (DMSO), positive (CC-885, 10 μM) and rescue controls (positive control plus 0.2 μM bortezomib) were also included at this stage. Cells were subsequently rinsed with PBS and fixed in 10% Formalin solution (50 μl, Sigma HT5011)) for 20 min at room temperature. Following three consecutive PBS washes (100 μL), cells were permeabilized in 0.1% Triton X-100 in PBS (Sigma 93443, 50 μl) for 15 min at room temperature. Following three further PBS washes, 50 μl blocking buffer (1% BSA, Sigma A4503, in PBS) was added for 45 min for signal-to noise reduction. Primary antibody (human GSPT1, Sigma HPA052488) was diluted in blocking buffer (dil.1/300, 35 μL/well) and incubated with the cells overnight at 4° C. After three PBS washes, Alexa-fluor 488 coupled secondary antibodies (Invitrogen, A32731, dil.1/1000), Alexa-fluor 647-Phalloidin (Invitrogen, A22287, dil.1/200) and DAPI (Thermo, #62248, dil.1/1000) were diluted in blocking buffer and incubated with the samples for 2 h at room temperature. After three final PBS washes, samples were conserved in 100 μL PBS in the dark, until measurement. Image acquisition was performed on the Operetta High-Content Imager (Perkin-Elmer). Fluorescence intensity of Alexa-F 5 luor 488 (GSPT1), Alexa-Fluor 647 (Actin) and DAPI (Nucleus) were measured. For the determination of GSPT1 DC₅₀ values, a custom algorithm implemented in the PerkinElmer image analysis software Harmony-Acapella® was developed. After user-defined setting of adjustment parameters, the analysis was run identically without human intervention for all image fields. DAPI staining of the nuclei was used to determine the location of cells using standard nuclei detection modules. Segmentation artifacts were removed by threshold-based filters for area, roundness and intensity. The outline of the cells was determined analogously from the sum of the normalized, smoothed DAPI and Actin channel, starting from each nucleus. The Alexa-Fluor 488 (GSPT1) signal intensity in each cell was finally measured, in order to obtain a Mean intensity per cell. GSPT1 degradation (DC₅₀) was calculated after normalization to controls and data import in CDD vault Database, using non-linear regression.

In Table 2, each compound is assigned a class (HTRF class) indicating the ability for Cereblon binding by means of their HTRF IC₅₀ values according to Example 3: A, B or C. According to the code, A represents an IC₅₀ value of <100 nM, B represents an IC₅₀ value>100 nM and <1000 nM, C represents an IC₅₀ value>1000 nM. IF Class assigns each compound a code indicating the ability for GSPT1 degradation: A, B or C. According to the code, A represents a DC₅₀ value of <30 nM, B represents a DC₅₀ value>30 nM and <300 nM and C represents a DC₅₀ value of >300 nM.

TABLE 2
HTRF Binding of compounds to CRBN
and Activity for GSPT1 degradation.

| Compound | HTRF Class | IF Class |
|---|---|---|
| 1 | B | C |
| 2 | B | C |
| 3 | B | A |
| 4 | B | A |
| 5 | B | A |
| 6 | B | B |
| 7 | B | C |
| 8 | B | C |
| 9 | B | A |
| 10 | A | C |
| 11 | B | A |
| 12 | A | B |
| 13 | B | C |
| 14 | B | C |
| 15 | B | C |
| 16 | B | B |
| 17 | B | C |
| 18 | B | A |
| 19 | A | C |
| 20 | B | C |
| 21 | B | C |
| 22 | B | C |
| 23 | B | C |
| 24 | B | C |
| 25 | B | C |
| 26 | B | C |
| 27 | B | B |
| 28 | B | B |
| 29 | B | A |
| 30 | B | B |
| 31 | B | B |
| 32 | B | B |
| 33 | B | B |
| 34 | — | C |
| 35 | — | C |
| 36 | B | C |
| 37 | — | C |
| 38 | — | A |
| 39 | — | B |
| 40 | C | A |
| 44 | — | B |
| 45 | — | C |
| 46 | B | — |
| 47 | B | C |
| 48 | A | C |
| 49 | B | C |
| 50 | B | C |
| 51 | B | C |
| 52 | B | B |

### EQUIVALENTS

While specific embodiments have been discussed, the above specification is illustrative and not restrictive. Many variations of the embodiments will become apparent to those skilled in the art upon review of this specification. The full scope of what is disclosed should be determined by reference to the claims, along with their full scope of equivalents, and the specification, along with such variations.

What is claimed is:

1. A compound of Formula (I): 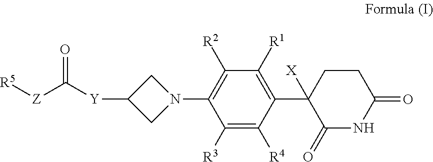
or a pharmaceutically acceptable salt thereof,
wherein:
X is H or deuterium;
each of Y and Z is independently C(R^{A})₂, NH, or C₃₋₁₀ cycloalkyl, provided that, when Y is NH, Z is CR^{A} or C₃₋₁₀ cycloalkyl, and when Y is CR^{A} or C₃₋₁₀ cycloalkyl, then Z is NH;
each of R¹, R², R³, and R⁴ is independently H or halogen;
R⁵ is C₁₋₆ alkyl, C₂₋₆ alkenyl, C₂₋₆ alkynyl, C₃₋₁₀ cycloalkyl, 3 to 10 membered heterocyclyl, aryl, or heteroaryl, wherein each of C₁₋₆ alkyl, C₂₋₆ alkenyl, C₂₋₆ alkynyl, C₃₋₁₀ cycloalkyl, aryl, and heteroaryl is optionally substituted with one or more substituents each independently selected from R⁶;
each occurrence of R⁶ is independently halogen, cyano, C₁₋₄ alkyl, C₁₋₆ alkoxy, C₃₋₁₀ cycloalkyl, 3 to 10 membered heterocyclyl, aryl, heteroaryl, —C(O)OR^{B}, or —OC(O)R^{C}, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more substituents each independently selected from R⁷,
or two R⁶ are joined to together to form a 3 to 10-membered ring optionally substituted with one or more substituents each independently selected from R⁷;
each occurrence of R⁷ is independently halogen, C₁₋₆ alkyl, 3 to 10 membered heterocyclyl, or aryl, wherein each of C₁₋₆ alkyl, 3 to 10 membered heterocyclyl, and aryl is optionally substituted with R⁸;
R⁸ is C₁₋₆ alkoxy; and
each occurrence of R^{A}, R^{B}, and R^{C} is independently selected from the group consisting of H, C₁₋₆ alkyl, and halogen.

2. The compound of claim 1, wherein X is H, Y is NH, Z is CH₂ or CF₂, and R¹ and R⁴ are halogen.

3. The compound of claim 1, wherein R⁵ is C₃₋₁₀ cycloalkyl, C₆₋₁₀ aryl, or 5-membered heteroaryl, or 6-membered heteroaryl.

4. The compound of claim 1, wherein R⁶ is halogen, cyano, C₁₋₆ alkyl, or C₁₋₆ alkoxy, wherein each of C₁₋₆ alkyl or C₁₋₆ alkoxy is optionally substituted with one or more occurrences of halogen, and R⁷ is halogen or C₁₋₆ alkyl.

5. The compound of claim 1, wherein m is 1, 2, or 3, n is 1, 2, or 3, and p is 1.

6. A compound of Formula (II): 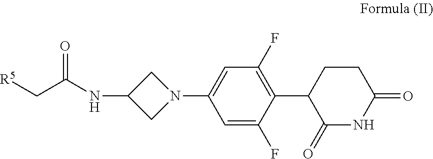
or a pharmaceutically acceptable thereof,
wherein:
R⁵ is heteroaryl, aryl, or C₃₋₁₀ cycloalkyl, wherein each of heteroaryl, aryl, and C₃₋₁₀ cycloalkyl is optionally substituted with one or more substituents each independently selected from R⁶;
each occurrence of R⁶ is independently halogen, cyano, C₁₋₆ alkyl, C₁₋₆ alkoxy, C₃₋₁₀ cycloalkyl, 3 to 10 membered heterocyclyl, phenyl, or 5-membered heteroaryl, or 6-membered heteroaryl, wherein each of C₁₋₆ alkyl, C₁₋₆ alkoxy, C₃₋₁₀ cycloalkyl, 3 to 10 membered heterocyclyl, phenyl, 5-membered heteroaryl, and 6-membered heteroaryl is optionally substituted with one or more substituents each independently selected from R⁷; and
each occurrence of R⁷ is independently halogen, or C₁₋₆ alkyl.

7. The compound of claim 6, wherein R⁵ is C₃₋₁₀ cycloalkyl, C₆₋₁₀ aryl, or 5 or 6-membered heteroaryl.

8. The compound of claim 6, wherein R⁶ is halogen, cyano, C₁₋₆ alkyl, or C₁₋₆ alkoxy, wherein each of C₁₋₆ alkyl or C₁₋₆ alkoxy is optionally substituted with one or more occurrences of halogen, and R⁷ is halogen or C₁₋₆ alkyl.

9. The compound of claim 6, wherein m is 1, 2, or 3, each occurrence of n is independently 1, 2, or 3, and p is 1.

10. A compound of Formula (III): 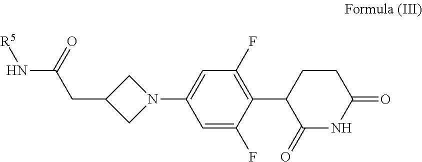
or a pharmaceutically acceptable thereof,
wherein:
R⁵ is heteroaryl, aryl, or C₃₋₁₀ cycloalkyl, wherein each of heteroaryl, aryl, and C₃₋₁₀ cycloalkyl is optionally substituted with one or more substituents each independently selected from R⁶;
each occurrence of R⁶ is independently halogen, cyano, C₁₋₆ alkyl, C₁₋₆ alkoxy, C₃₋₁₀ cycloalkyl, 3 to 10 membered heterocyclyl, phenyl, or 5-membered heteroaryl, or 6-membered heteroaryl, wherein each of C₁₋₆ alkyl, C₁₋₆ alkoxy, C₃₋₁₀ cycloalkyl, 3 to 10 membered heterocyclyl, phenyl, 5-membered heteroaryl, and 6-membered heteroaryl is optionally substituted with one or more substituents each independently selected from R⁷; and
each occurrence of R⁷ is independently halogen, or C₁₋₆ alkyl.

11. The compound of claim 10, wherein R⁵ is C₃₋₁₀ cycloalkyl, C₆₋₁₀ aryl, 5-membered heteroaryl, or 6-membered heteroaryl.

12. The compound of claim 10, wherein R⁶ is halogen, cyano, C₁₋₆ alkyl, or C₁₋₆ alkoxy, wherein each of C₁₋₆ alkyl or C₁₋₆ alkoxy is optionally substituted with one or more occurrences of halogen, and R⁷ is halogen or C₁₋₆ alkyl.

13. The compound of claim 10, wherein m is 1, 2, or 3, each occurrence of n is independently 1, 2, or 3, and p is 1.

14. A compound selected from the group consisting of: 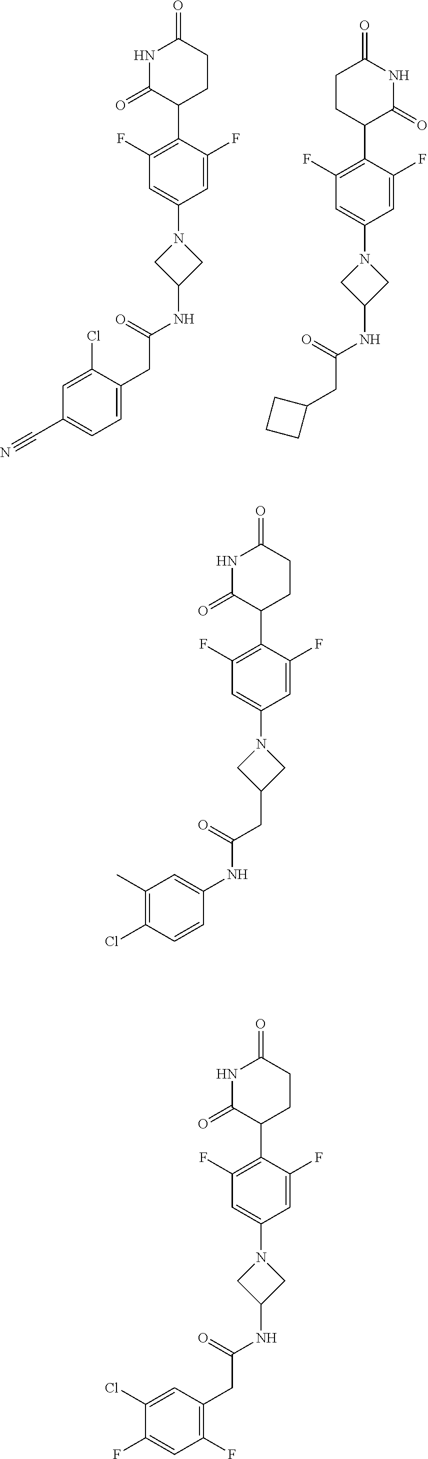 [SMILES: N#Cc1ccc(CC(=O)NC2CN(c3cc(F)c(C4CCC(=O)NC4=O)c(F)c3)C2)c(Cl)c1 ; O=C1CCC(c2c(F)cc(N3CC(NC(=O)CC4CCC4)C3)cc2F)C(=O)N1 ; Cc1cc(NC(=O)CC2CN(c3cc(F)c(C4CCC(=O)NC4=O)c(F)c3)C2)ccc1Cl ; O=C1CCC(c2c(F)cc(N3CC(NC(=O)Cc4cc(Cl)c(F)cc4F)C3)cc2F)C(=O)N1] 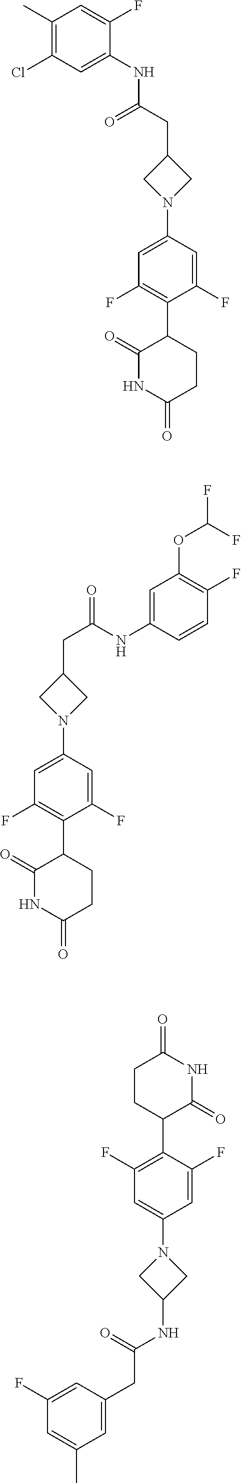 [SMILES: Cc1cc(F)c(NC(=O)CC2CN(c3cc(F)c(C4CCC(=O)NC4=O)c(F)c3)C2)cc1Cl ; O=C1CCC(c2c(F)cc(N3CC(CC(=O)Nc4ccc(F)c(OC(F)F)c4)C3)cc2F)C(=O)N1 ; Cc1cc(F)cc(CC(=O)NC2CN(c3cc(F)c(C4CCC(=O)NC4=O)c(F)c3)C2)c1] 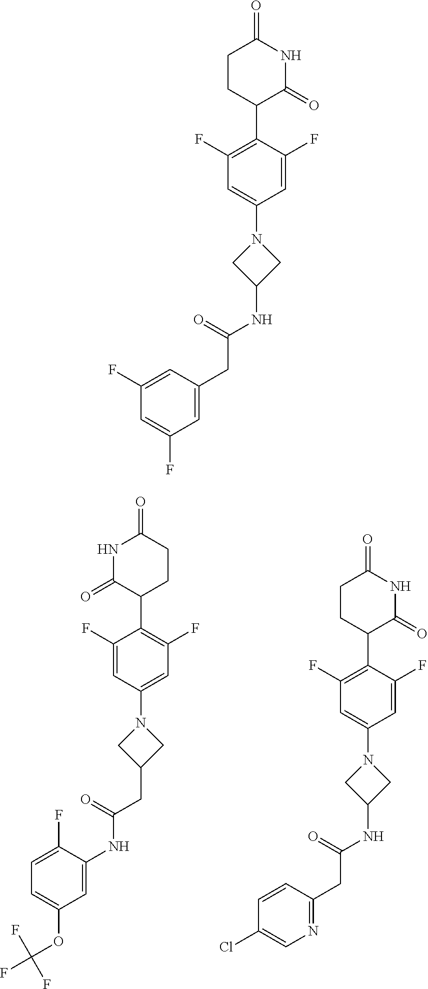 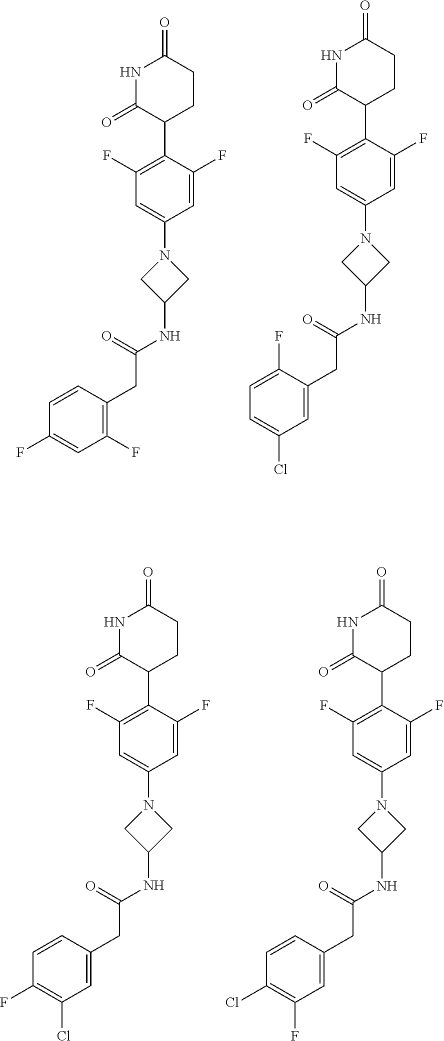 [SMILES: O=C1CCC(c2c(F)cc(N3CC(NC(=O)Cc4ccc(F)cc4F)C3)cc2F)C(=O)N1 ; O=C1CCC(c2c(F)cc(N3CC(NC(=O)Cc4cc(Cl)ccc4F)C3)cc2F)C(=O)N1 ; O=C1CCC(c2c(F)cc(N3CC(NC(=O)Cc4ccc(F)c(Cl)c4)C3)cc2F)C(=O)N1 ; O=C1CCC(c2c(F)cc(N3CC(NC(=O)Cc4ccc(Cl)c(F)c4)C3)cc2F)C(=O)N1] 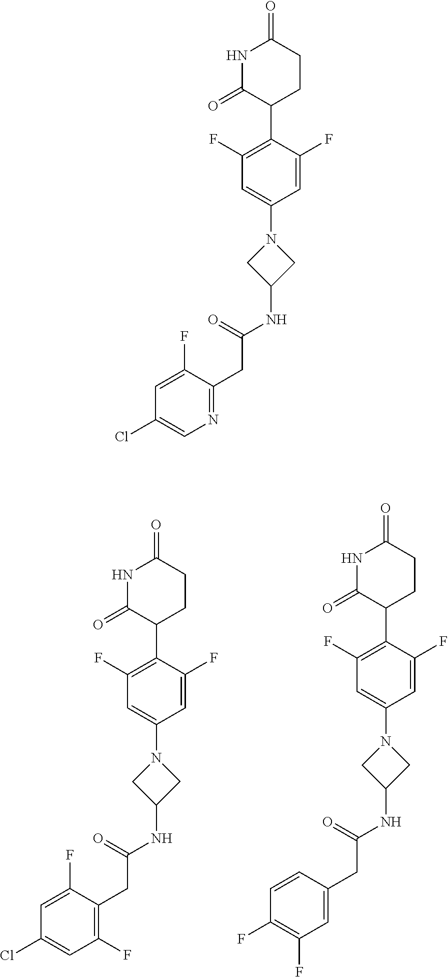 [SMILES: O=C1CCC(c2c(F)cc(N3CC(NC(=O)Cc4ncc(Cl)cc4F)C3)cc2F)C(=O)N1 ; O=C1CCC(c2c(F)cc(N3CC(NC(=O)Cc4c(F)cc(Cl)cc4F)C3)cc2F)C(=O)N1 ; O=C1CCC(c2c(F)cc(N3CC(NC(=O)Cc4ccc(F)c(F)c4)C3)cc2F)C(=O)N1] 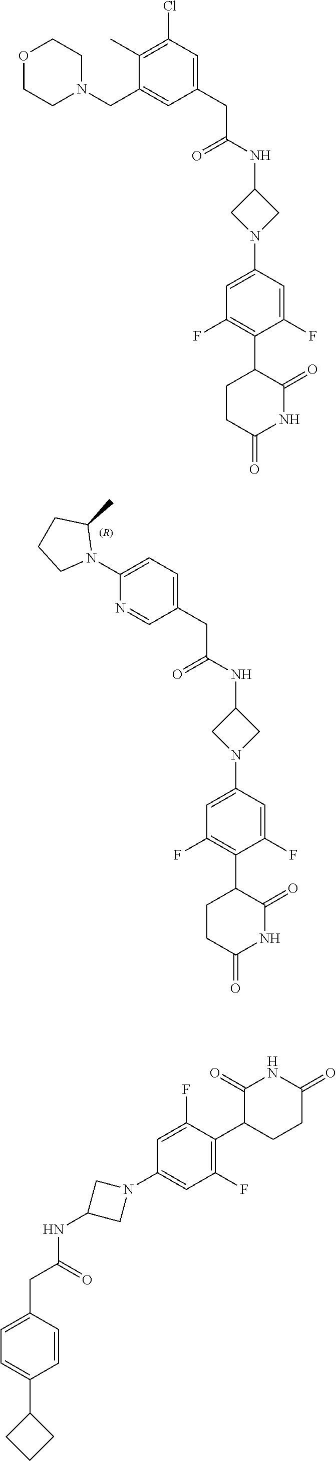 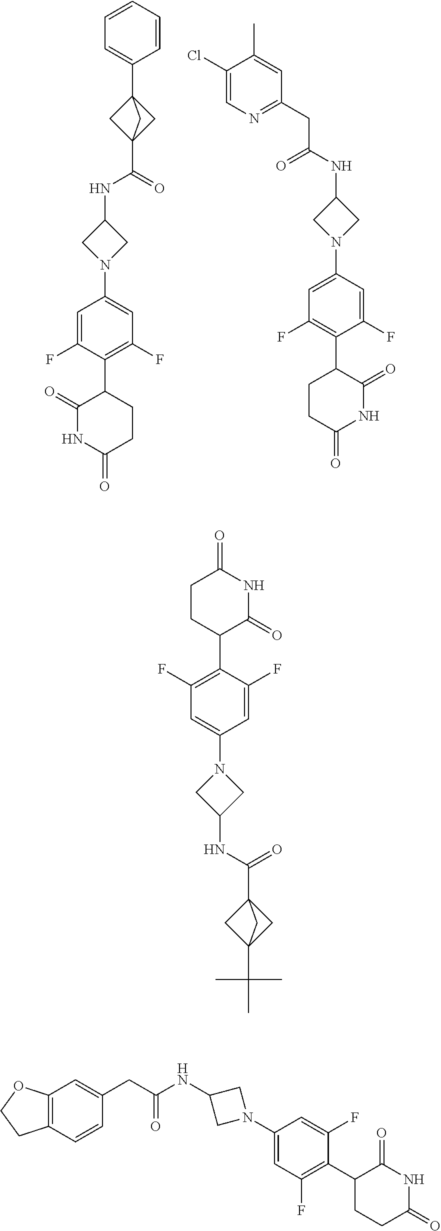 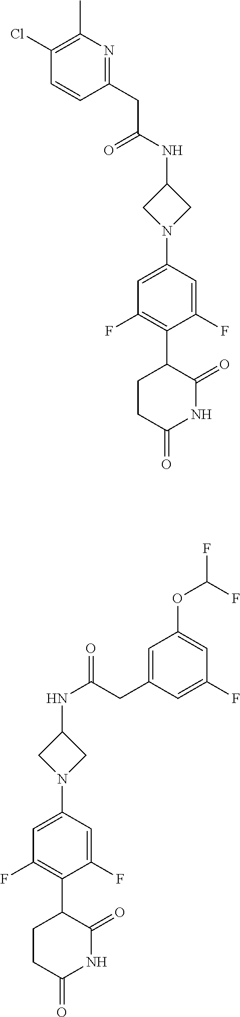 [SMILES: Cc1nc(CC(=O)NC2CN(c3cc(F)c(C4CCC(=O)NC4=O)c(F)c3)C2)ccc1Cl ; O=C1CCC(c2c(F)cc(N3CC(NC(=O)Cc4cc(F)cc(OC(F)F)c4)C3)cc2F)C(=O)N1] 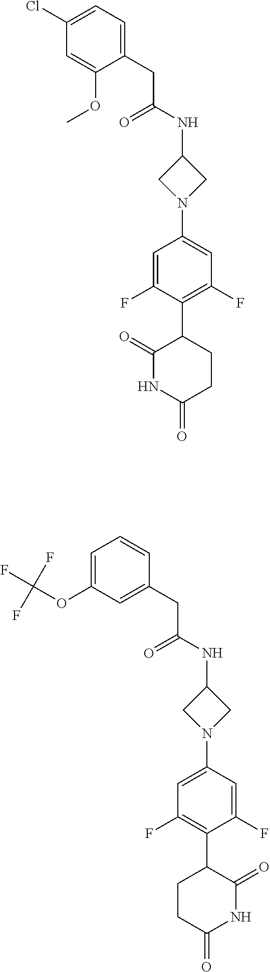 [SMILES: COc1cc(Cl)ccc1CC(=O)NC1CN(c2cc(F)c(C3CCC(=O)NC3=O)c(F)c2)C1 ; O=C1CCC(c2c(F)cc(N3CC(NC(=O)Cc4cccc(OC(F)(F)F)c4)C3)cc2F)C(=O)N1] 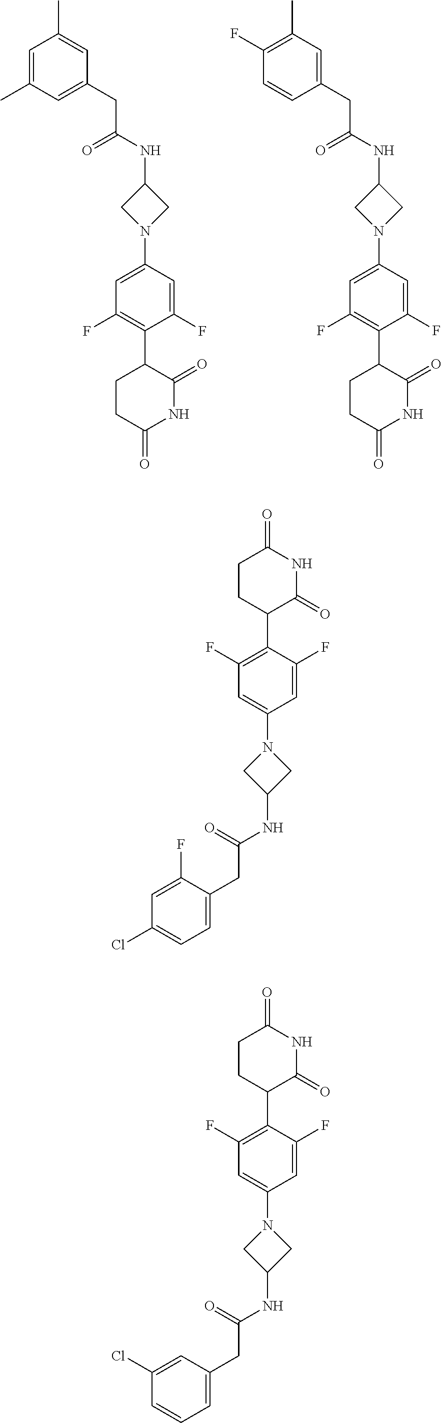 [SMILES: Cc1cc(C)cc(CC(=O)NC2CN(c3cc(F)c(C4CCC(=O)NC4=O)c(F)c3)C2)c1 ; Cc1cc(CC(=O)NC2CN(c3cc(F)c(C4CCC(=O)NC4=O)c(F)c3)C2)ccc1F ; O=C1CCC(c2c(F)cc(N3CC(NC(=O)Cc4ccc(Cl)cc4F)C3)cc2F)C(=O)N1 ; O=C1CCC(c2c(F)cc(N3CC(NC(=O)Cc4cccc(Cl)c4)C3)cc2F)C(=O)N1] 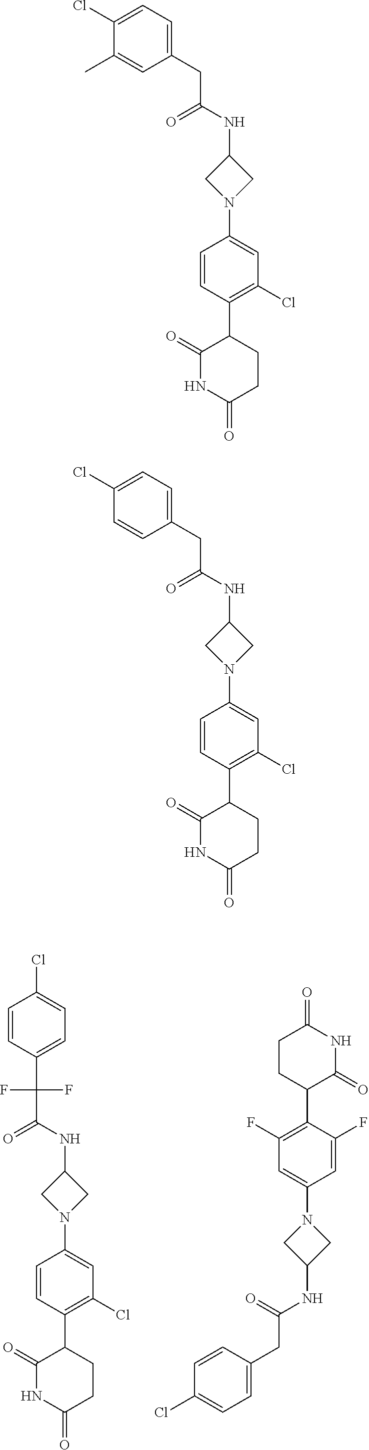 [SMILES: Cc1cc(CC(=O)NC2CN(c3ccc(C4CCC(=O)NC4=O)c(Cl)c3)C2)ccc1Cl ; O=C1CCC(c2ccc(N3CC(NC(=O)Cc4ccc(Cl)cc4)C3)cc2Cl)C(=O)N1 ; O=C1CCC(c2ccc(N3CC(NC(=O)C(F)(F)c4ccc(Cl)cc4)C3)cc2Cl)C(=O)N1 ; O=C1CCC(c2c(F)cc(N3CC(NC(=O)Cc4ccc(Cl)cc4)C3)cc2F)C(=O)N1] 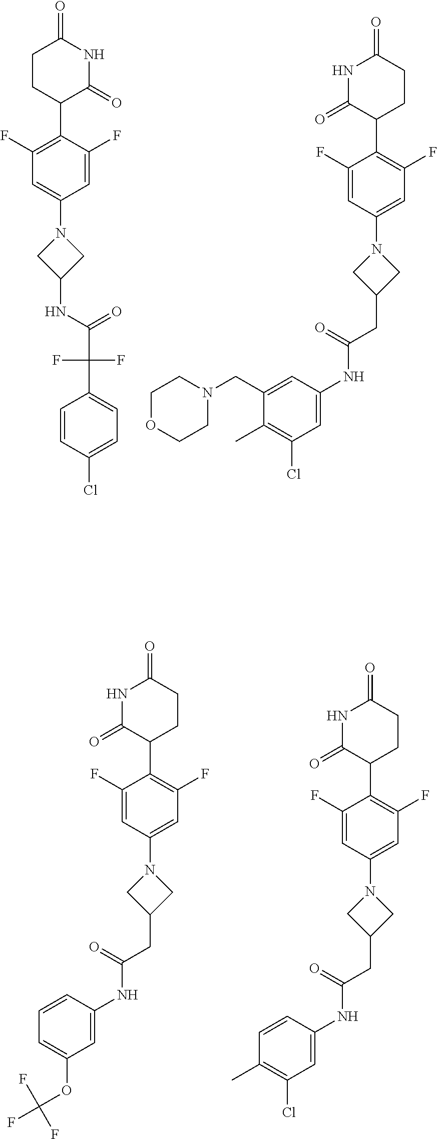 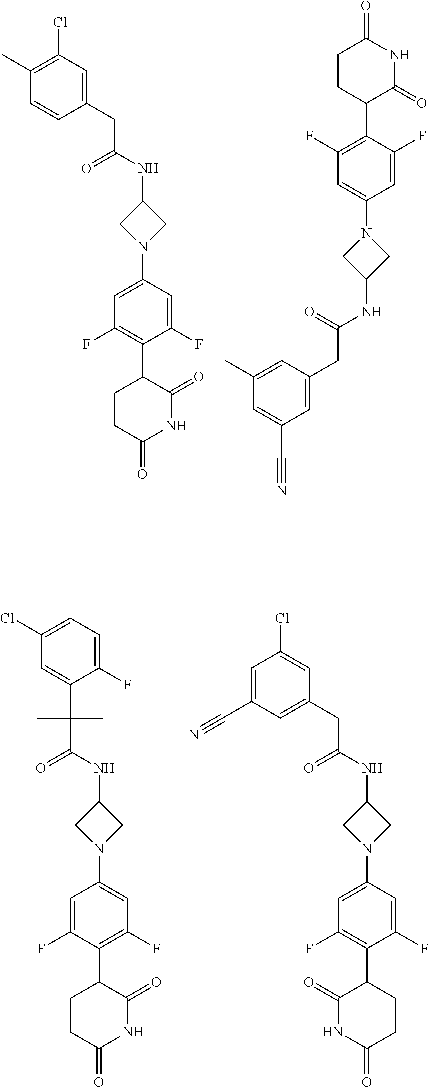 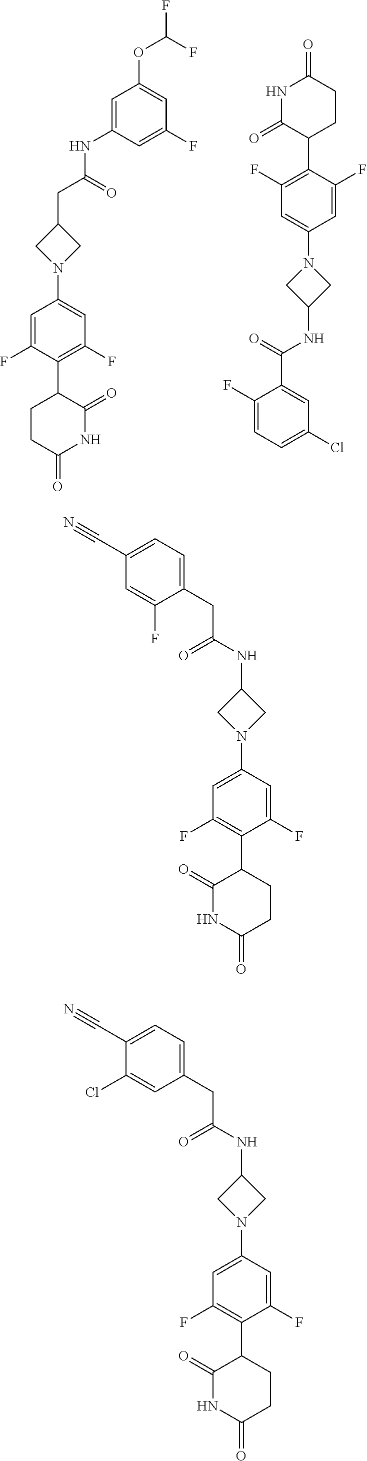 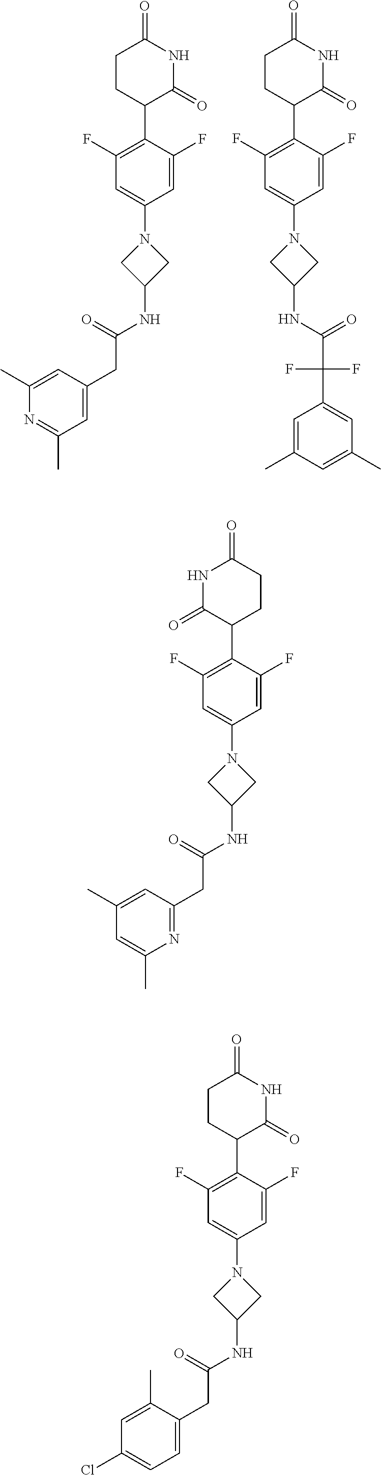 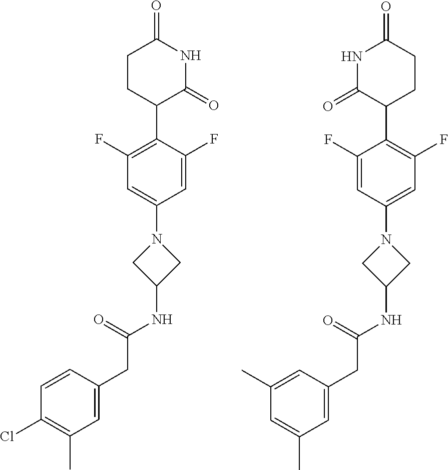 [SMILES: Cc1cc(CC(=O)NC2CN(c3cc(F)c(C4CCC(=O)NC4=O)c(F)c3)C2)ccc1Cl ; Cc1cc(C)cc(CC(=O)NC2CN(c3cc(F)c(C4CCC(=O)NC4=O)c(F)c3)C2)c1]
and pharmaceutically acceptable salts thereof.

15. A pharmaceutical composition comprising the compound of claim 1, or pharmaceutically acceptable salt thereof, and a pharmaceutically acceptable excipient.

16. A method of degrading GSPT1 in a subject suffering from cancer, comprising administering to the subject an effective amount of the compound of claim 1.

17. A method of treating cancer in a subject in need thereof, comprising administering to the subject a therapeutically effective amount of the compound of claim 1.
